(19) United States
(12) Patent Application Publication
(10) Pub. No.: US 2026/0232824 A1
(43) Pub. Date: Aug. 13, 2026

(54) ANTI-CD180 BINDING MOLECULES AND USES THEREOF

(71) Applicant: Champions Oncology, Inc., Hackensack, NJ (US)

(72) Inventors: Garima KAUSHIK, Rockville, MD (US); Marina BELL, Rockville, MA (US); Kakajan KOMUROV, Rockville, MD (US); Michael Ritchie, Rockville, MD (US)

(73) Assignee: Champions Oncology, Inc., Hackensack, NJ (US)

(21) Appl. No.: 19/152,391

(22) PCT Filed: Feb. 2, 2024
(86) PCT No.: PCT/US2024/014284

§ 371 Date: Jul. 30, 2025

Related U.S. Application Data
(60) Provisional application No. 63/483,069, filed on Feb. 3, 2023.

(51) Int. Cl. A61K 47/68; A61P 35/02; C07K 16/28

(52) U.S. Cl. CPC A61K 47/68035; A61K 47/6849; A61K 47/6889; A61P 35/02; C07K 16/2896

(57) ABSTRACT

The present disclosure provides anti-CD180 binding molecules and uses thereof. In one embodiment, the anti-CD180 binding molecules are anti-CD180 antibodies. Also provided are anti-CD180 antibody-drug conjugates (ADCs) comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload. The anti-CD180 antibodies or the antigen binding fragments thereof, and the ADCs comprising the anti-CD180 antibodies, or the antigen binding fragments thereof are useful in treating diseases, such as acute myeloid leukemia, mantle cell lymphoma, multiple myeloma. follicular lymphoma, B-acute lymphoblastic leukemia, or diffuse large B-cell lymphoma.

### SEQUENCE LISTING INCORPORATION

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Jan. 30, 2024, is entitled “P-620104-USP_ST26.xml”, and is 236,802 bytes in size.

### FIELD OF THE INVENTION

The present disclosure relates in general to the field of antibodies and antibody-drug conjugates (ADCs). In one embodiment, the present disclosure provides anti-CD180 binding molecules and uses thereof. The present disclosure relates in particular to an anti-CD180 antibody-drug conjugate (ADC) comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload.

### BACKGROUND OF THE INVENTION

Toll-like receptors (TLRs) are pattern recognition receptors that bind a variety of microbial products such as microbial membrane lipids or nucleic acids. Antigen presenting cells (APC) including B cells, DCs and macrophages express multiple TLRs that are bound by pathogens to activate NF-κB and MAP kinase pathways, resulting in expression of costimulatory molecules and cytokine secretion. While TLRs are important for defense against infectious disease, increasing evidence suggests that TLRs also function as regulators of immune responses in cancer, autoimmune disease, and transplantation.

CD180 is an orphan member of the TLR family. CD180 is the human analogue of the murine surface receptor RP105 sharing 74% sequence homology. CD180 receptor was originally discovered on murine B cells and identified on human B cells by Bgp95 mAb. It is a membrane-associated receptor with a molecular weight of 105 kD. The extracellular portion of CD180 consists of tandem repeats of leucine-rich motif separated from the single transmembrane domain by a carboxy-flanking region thought to be involved in processes such as cell adhesion or receptor-ligand interaction. The presence of conserved cysteine residues in the carboxy-flanking region in CD180 is a common feature with other TLRs. CD180 is expressed on antigen presenting cells such as human B lymphocytes, monocytes and dendritic cells. Histological studies have shown CD180 is mainly expressed on mature B cells in mantle zones whereas its expression in germinal center cells is either very low or negative. CD180 forms a complex with MD-1, which is structurally related to TLR4/MD-2 complex, to elicit responses to bacterial LPS.

CD180 is homologous to TLR4 but lacks its intracellular TLR-like domain with 61% sequence similarity in the extracellular domain. These two receptors use two distinct signaling pathways. LPS binding to CD180/MD1 induces Lyn activation and CD19 phosphorylation, whereas LPS binding to TLR4/MD-2 activates MyD88/IRAK and MyD88-independent Toll-interleukin 1 receptor (TIR) domain-containing adapter protein (TIRAP) pathways to activate JNK and NFκB.

Several studies have supported the importance of CD180 in B cell survival, activation, proliferation and/or differentiation. Although TLRs have an intracellular TIR domain and an adaptor molecule Myd88 which activates the various downstream protein kinases, CD180 does not have a functional cytoplasmic signaling domain, and it cannot independently propagate an intracellular signal. It must, therefore, recruit or converge with other receptor pathways. CD180-mediated signaling pathway was independent of MyD88 expression, and its functioning is regulated by CD19. In mice, CD180 ligation recruits CD19 into lipid rafts and induces phosphorylation of CD19 which in turns amplifies Src kinase Lyn activity. Phosphorylated CD19 recruits Vav, which is important to act upstream of JNK pathway. Therefore, CD19 may mediate the interaction between Lyn and Vav in CD180-mediated signaling. On the other hand, PI3K and NF-κB activation by CD180 ligation were shown to be independent of CD19.

The contribution of the microenvironment to the pathogenesis of lymphoproliferative disorders (LPDs) is well recognized. Next-generation sequencing studies of LPDs have described several molecular lesions in signaling pathways activated by downstream microenvironmental stimuli, including Toll-like receptor (TLR) signaling. CD180 modulates the ligand-induced activity of TLR2 and TLR4 and may positively regulate CD19 signaling in murine models. Like many TLRs, CD180 appears to be more highly expressed by memory B cells than naïve B cells. Flow cytometry (FCM) analysis of peripheral blood (PB) has reported that CD180 expression by B cells in marginal zone lymphoma (MZL) is strong, whereas CD180 expression level is markedly lower in other LPDs such as chronic lymphocytic leukemia (CLL) and mantle cell lymphoma (MCL). These results indicate that CD180 can be considered as a single positive robust marker of MZL and should be included in flow cytometry panels for the diagnosis of mature B cell neoplasms.

Accumulated evidence also shows that CD180 is tightly associated with the pathogenesis of autoimmune diseases. CD180 can modulate the functions of antigen-presenting cells and regulate the development of collagen-induced arthritis. The population of CD180-negative B cell significantly increases in systemic lupus erythematosus (SLE) patients and it changes in parallel with SLE disease activity. Abnormal activation of macrophages and DCs have been shown to contribute to the pathogenesis of SLE. It has been reported that ligation of CD180 significantly inhibited TLR7- and TLR9-mediated activation of macrophages and DCs through the Lyn-SHP-1/2 axis, and treatment with anti-CD180 antibodies efficiently ameliorated the lupus-symptoms in lupus-prone mice. Taken together, these results indicate that CD180 plays a critical role in regulating the activation of TLR7 and TLR9 pathways in macrophages and DCs, suggesting that CD180 may be used as a potential therapeutic target for SLE treatment.

Thus, there is a need to further develop anti-CD180 binding molecules that would facilitate the studies and regulation of CD180-mediated immune responses in B cells and lymphoproliferative disorders.

### SUMMARY OF THE INVENTION

The present disclosure describes the isolation of a panel of anti-CD180 binding molecules and uses thereof. In one embodiment, the present disclosure provides an isolated anti-CD180 binding molecule comprising a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 comprises the amino acid sequences as shown in Table 1, and the set of corresponding LCDR1, LCDR2 and LCDR3 comprises the amino acid sequences as shown in Table 2.

In one embodiment, the anti-CD180 binding molecule comprises a heavy chain variable region and a light chain variable region having the sequences as disclosed herein.

In one embodiment, the anti-CD180 binding molecule comprises a heavy chain and a light chain having the sequences as disclosed herein.

In one embodiment, the anti-CD180 binding molecule comprises an IgG, a Fv, a scFv, a Fab, a F(ab′)2, a minibody, a diabody, a triabody, a nanobody, a bispecific antibody, a single domain antibody, or a chimeric antigen receptor. In one embodiment, the IgG is IgG1, IgG2, IgG3, or IgG4. In an embodiment, the bispecific anti-CD180 antibody is a bi-paratopic antibody, i.e., a bispecific antibody that targets two non-overlapping epitopes on the same target antigen. In some embodiments, the bispecific antibody binds to a CD180 and a CD123 (“a CD180×CD123 bi-specific antibody”). In an embodiment, the bispecific anti-CD180 antibody comprises the variable heavy chain (VH) and variable light chain (VL) regions and/or the CDRs disclosed herein and the VH, VL, and/or the CDRs combinations disclosed herein that have binding specificity to CD180, i.e., comprises a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each comprise the amino acid sequences disclosed herein. In another embodiment, there are provided isolated polynucleotide sequences encoding the anti-CD180 binding molecules disclosed herein. In another embodiment, the present disclosure provides a vector comprising the polynucleotide sequence disclosed herein. In another embodiment, the present disclosure provides a host cell comprising the vector disclosed herein.

In one embodiment, the present disclosure provides a composition comprising a pharmaceutically acceptable carrier and any one of the anti-CD180 binding molecules disclosed herein. In one embodiment, the composition comprises antibody drug conjugates. In one embodiment, the antibody drug conjugates comprise tesirine. In some embodiments, the antibody drug conjugates comprise deruxtecan.

In another embodiment, the present disclosure provides a composition comprising any of the polynucleotide sequences disclosed herein. In one embodiment, the polynucleotide in such composition comprises an expression vector for expressing the anti-CD180 binding molecule in a cell.

The present disclosure provides an anti-CD180 antibody-drug conjugate (ADC) comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the anti-CD180 antibody or antigen-binding fragment thereof comprises a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each comprise the amino acid sequences: HCDR1, HCDR2 and HCDR3 of SEQ ID NOs: 1-3 and LCDR1, LCDR2 and LCDR3 of SEQ ID NOs: 4-6; HCDR1, HCDR2 and HCDR3 of SEQ ID NOs: 1-3 and LCDR1, LCDR2 and LCDR3 of SEQ ID NOs: 4, 5, and 15; HCDR1, HCDR2 and HCDR3 of SEQ ID NOs: 1, 24, and 25 and LCDR1, LCDR2 and LCDR3 of SEQ ID NOs: 4, 5, and 26; HCDR1, HCDR2 and HCDR3 of SEQ ID NOs: 1, 35, and 25 and LCDR1, LCDR2 and LCDR3 of SEQ ID NOs: 4, 5, and 26; HCDR1, HCDR2 and HCDR3 of SEQ ID NOs: 1, 44, and 25 and LCDR1, LCDR2 and LCDR3 of SEQ ID NOs: 4, 5, and 45; HCDR1, HCDR2 and HCDR3 of SEQ ID NOs: 54-56 and LCDR1, LCDR2 and LCDR3 of SEQ ID NOs: 57-59; HCDR1, HCDR2 and HCDR3 of SEQ ID NOs: 68-70 and LCDR1, LCDR2 and LCDR3 of SEQ ID NOs: 57, 58, and 71; HCDR1, HCDR2 and HCDR3 of SEQ ID NOs: 1, 24, and 25 and LCDR1, LCDR2 and LCDR3 of SEQ ID NOs: 80-82; HCDR1, HCDR2 and HCDR3 of SEQ ID NOs: 1, 35, and 25 and LCDR1, LCDR2 and LCDR3 of SEQ ID NOs: 91-93; HCDR1, HCDR2 and HCDR3 of SEQ ID NOs: 1, 102, and 25 and LCDR1, LCDR2 and LCDR3 of SEQ ID NOs: 4, 103, and 6; HCDR1, HCDR2 and HCDR3 of SEQ ID NOs: 1, 24, and 25 and LCDR1, LCDR2 and LCDR3 of SEQ ID NOs: 112, 5, and 113; HCDR1, HCDR2 and HCDR3 of SEQ ID NOs: 1, 35, and 25 and LCDR1, LCDR2 and LCDR3 of SEQ ID NOs: 4-6; HCDR1, HCDR2 and HCDR3 of SEQ ID NOs: 1, 44, and 25 and LCDR1, LCDR2 and LCDR3 of SEQ ID NOs: 4, 5, and 130; or HCDR1, HCDR2 and HCDR3 of SEQ ID NOs: 1, 24, and 25 and LCDR1, LCDR2 and LCDR3 of SEQ ID NOs: 4-6.

The present disclosure also provides an anti-CD180 antibody-drug conjugate (ADC) comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the anti-CD180 antibody or antigen-binding fragment thereof comprises a heavy chain variable region and a light chain variable region, said heavy chain variable region and light chain variable region comprise the amino acid sequences of SEQ ID NOs:7-8, SEQ ID NOs:16-17, SEQ ID NOs:27-28, SEQ ID NOs:36-37, SEQ ID NOs:46-47, SEQ ID NOs:60-61, SEQ ID NOs:72-73, SEQ ID NOs:83-84, SEQ ID NOs:94-95, SEQ ID NOs:104-105, SEQ ID NOs:114-115, SEQ ID NOs:122-123, SEQ ID NOs:131-132, or SEQ ID NOs:139-140.

The present disclosure provides an anti-CD180 antibody-drug conjugate (ADC) comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the anti-CD180 antibody or antigen-binding fragment thereof comprises a heavy chain and a light chain, said heavy chain and light chain comprise the amino acid sequences of SEQ ID NOs: 11-12, SEQ ID NOs:20-21, SEQ ID NOs:31-32, SEQ ID NOs:40-41, SEQ ID NOs:50-51, SEQ ID NOs:64-65, SEQ ID NOs:76-77, SEQ ID NOs:87-88, SEQ ID NOs:98-99, SEQ ID NOs:108-109, SEQ ID NOs:118-119, SEQ ID NOs:126-127, SEQ ID NOs:135-136, or SEQ ID NOs:143-144.

In a particular embodiment of the provided ADCs, the cytotoxic drug payload comprises a ADC drug-linker conjugate tesirine (SG3249), the tesirine comprising a cytotoxic drug payload, the cytotoxic drug payload comprising pyrrolobenzodiazepine dimer cytotoxic DNA-alkylating agent (SG3199), wherein the tesirine is conjugated to the CD180-high expressing tumor-targeting monoclonal antibody or antigen-binding fragment thereof via a cleavable linker moiety.

In an embodiment of the provided ADCs, the ADC comprises an ADC drug-linker conjugate deruxtecan, the deruxtecan comprising a cleavable linker, a self-immolative amino methylene spacer, and a cytotoxic drug payload, the cytotoxic drug payload comprising a topoisomerase 1 inhibitor payload, wherein the topoisomerase 1 inhibitor payload is a derivative of exatecan (DX-8951), wherein the deruxtecan is conjugated to the CD180-high expressing tumor-targeting monoclonal antibody or antigen-binding fragment thereof via the cleavable linker. In an embodiment, the cleavable linker is a glycine-glycine-phenylalanine-glycine tetrapeptide-based linker.

In another embodiment, the present disclosure provides a method of modulating CD180-mediated immune responses in a subject, comprising the step of administering to the subject a composition comprising any one of the anti-CD180 binding molecules disclosed herein. In one embodiment, the composition comprises the herein provided antibody drug conjugates. In another embodiment, the present disclosure provides a method of modulating CD180-mediated immune responses in a subject, comprising the step of administering to the subject a composition comprising any one of the polynucleotide sequences disclosed herein.

In another embodiment, the present disclosure provides a method of treating a disease in a subject, comprising the step of administering to the subject a composition comprising any one of the anti-CD180 binding molecules disclosed herein. In one embodiment, the composition comprises antibody drug conjugates. In one embodiment, the antibody drug conjugates comprise tesirine (an ADC drug-linker conjugate). In certain embodiments, antibody drug conjugates comprise deruxtecan (an ADC drug-linker conjugate). In some embodiments, the present disclosure provides a method of treating a disease in a subject, comprising the step of administering to the subject a composition comprising any one of the polynucleotide sequences disclosed herein.

These and other aspects of the invention will be appreciated from the ensuing descriptions of the figures and detailed description of the invention.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-IC show the cell binding properties of the anti-CD180 antibodies. All CHAMP mAbs were subjected to binding assays via cell-based ELISA. FIG. 1A: Antibodies were screened at 100 nm and 30 nM in a cell-based ELISA using HEK293 expressing CD180/MD-1. All CHAMP mAbs showed significant binding to the ELISA. P84, mouse IgG, and 2nd only mouse were negative controls. MAS-17729 and MHR3-11 were commercial positive control antibodies. FIG. 1B: Antibodies were screened at 100 nm in cell-based ELISA using HEK293 expressing MD-1 only. No CHAMP mAbs showed significant binding to the ELISA. P84, mouse IgG, 2^{nd} only mouse, MAS-17729 and MHR3-11 were negative control antibodies. FIG. 1C: Antibodies were screened at 100 nm in cell-based ELISA using HEK293 parental cell line. No CHAMP-mAbs showed significant binding to the cells. P84, Mouse IgG, 2nd only mouse, MAS-17729 and MHR3-11 were negative control antibodies.

FIG. 2 shows four CHAMP mAbs were subjected to EC50 assessment via cell-based ELISA using HEK293 cells expressing human CD180/MD-1. MAS-17729 and MHR-73-11 were commercial positive controls.

FIGS. 3A-3C show intrinsic binding affinity of the antibodies as determined by Surface Plasmon Resonance (SPR) assay. Antibody was captured in a bivalent format and soluble human CD180 from Sino Biologics (FIG. 3A), Creative Biomart (FIG. 3B), or soluble rhesus CD180 (FIG. 3C) was passed over the captured antibody. Only ChampmAb-006 and ChampmAb-007 showed binding to soluble the protein, indicating that the epitopes for all other Champ mAbs are masked when CD180 is expressed as a soluble protein. Kon, koff and KD are indicated for each condition.

FIG. 4 shows results of Differential Scanning Fluorimetry (DSF) and Turbidity assays that were used to predict an antibody's thermal aggregation rate. DSF and Turbidity assays were performed for all antibodies. In all assays, the antibodies exhibited an acceptable melting temperature (Tm) above 65° C.

FIG. 5 shows results of Affinity Capture Self-Interaction Nanoparticle Spectroscopy (AC-SINS) assay that was used to test how likely an antibody is to interact with itself. All CHAMP antibodies exhibited a favorable profile below a shift of 11 nM, indicating a low propensity to self-interact. FDA-approved antibodies Infiximab, Pembrolizumab, Rituximab were used as benchmarks for the assay.

FIG. 6 shows results of size-exclusion chromatography. All CHAMP antibodies show a high level of purity after production (>95%) before and after a freeze thaw cycle (1×F/T).

FIG. 7 shows results of capillary electrophoresis sodium dodecyl sulfate (CE-SDS), an analytical method to assess the purity of proteins. All CHAMP mABs tested under this method show a high level of purity (>95%) after production.

FIG. 8 shows results of Baculovirus particles ELISA that tests the propensity of an antibody to interact with proteins in a non-specific manner. All CHAMP mAbs show a low propensity for non-specific, polyreactive binding.

FIG. 9 shows one embodiment of antibody conjugation to the linker-payload tesirine.

FIGS. 10A-10B shows ex vivo cytotoxicity of ADCs in primary acute myeloid leukemia. An ex vivo cell killing assay with primary leukapheresis-derived acute myeloid leukemia (AML) was performed. Primary cultures were established in a 96-well plate format, with 54-test wells utilized. Test agents were administered for a period of 120 hours incubation before Cell Titer Glo was administered to test cell viability. The half maximal inhibitory concentration (IC₅₀) was calculated and plotted against the RNA expression level (Transcript Per Million, TPM), as determined by RNAseq.

FIG. 11 shows inhibition of ex vivo cytotoxicity of ADCs in primary acute myeloid leukemia. An ex vivo cell killing assay with primary leukapheresis-derived AML was performed. Primary cultures were established in a 96-well plate format, with 54-test wells utilized. MHR73-11 was added to cells at a concentration of 300 ng/mL for 10 minutes. Test agents were administered at a concentration of 300 ng/mL for a period of 120 hours incubation before Cell Titer Glo was administered to test cell viability.

FIG. 12 shows ex vivo cytotoxicity of ADCs in primary mantle cell lymphoma. An ex vivo cell killing assay with primary leukapheresis-derived or blood-derived mantle cell lymphoma (MCL) was performed. Primary cultures were established in a 96-well plate format, with 54-test wells utilized. Test agents were administered for a period of 120 hours incubation before Cell Titer Glo was administered to test cell viability. The half maximal inhibitory concentration (IC₅₀) was calculated.

FIGS. 13A-13D show the CD180 target overview. FIG. 13A shows CD180 surface expression is enriched in Primary AML: protein expression distribution in Primary AML CD180 proteomics correlates with cell surface expression in Primary AML, and CD180 is elevated in Primary AML over normal immune cells; thus; cytogenetic abnormalities can be used as biomarkers for first-in-human study (FIH) studies. FIG. 13B shows CD180 expression in Primary normal bone marrow (BM) suggests an improved therapeutic index (TI) over CD133 ADCs. CD180 expression is lower than CD133 in normal hematopoietic stem cells (HSCs) and progenitor cells and expression is highest in mature B and plasmacytoid dendritic (pDC) cells. FIG. 13C shows CD180 is expressed on leukemic stem cells (LSC) and progenitor cells in Primary AML which will eliminate measurable residual disease (MRD). FIG. 13D shows enriched inflammatory signaling is observed in CD180-high tumors. Inflammatory AML provides a survival niche for LSCs. Primary AML with enriched CD180 expression also show elevated genes involved in inflammatory processes. Pathways involved in the innate immune response and response to oxidative stress are enriched in CD180 high primary AML.

FIGS. 14A-14G show the characteristics of anti-CD180 antibody leads CHAMPmAB-001 and CHAMPmAB-014: melting temperature, self-interaction, freeze thaw stability, purity (FIGS. 14A-14F); a comparison of CHAMPmAB-001 and CHAMPmAB-014 with isotype controls (ATX-P-84, and mIgG1), with secondary controls (hIgG1, hu2′Ab only and mu2′Ab only) and with no stain (a control sample) for binding to humanCD180/MD1 (positive) cells, MD1 (negative) cells, and HEK parental cells (negative) (FIG. 14F); and CHAMPmAB-001 (6 nM) and CHAMPmAB-014 (4 nM) half maximal effective concentration (EC₅₀) on HEK293 CD180/MD-1 cells (FIG. 14G).

FIGS. 15A-15C show conjugation of anti-CD180 antibody lead CHAMPmAB-001 to tesirine compared to conjugation of an isotype IgG1 to tesirine. FIG. 15A shows a schematic of an embodiment of an ADC of anti-CD180 antibody conjugated to the linker-payload tesirine (×2). FIG. 15B shows a drug-to-antibody ratio (DAR) of 2.1, i.e., the average number of drug conjugated to an anti-CD180 antibody according to the present disclosure, CHAMPmAB-001, conjugated to the linker-payload tesirine (×2) (CO-ADC-001). FIG. 15C shows a DAR of 2.2 of an isotype IgG1 conjugated to the linker-payload tesirine (×2) (CO-ADC-003).

FIGS. 16A-16C show conjugation of anti-CD180 antibody lead CHAMPmAB-004 to deruxtecan compared to conjugation of an isotype IgG1 to deruxtecan. FIG. 16A shows a schematic of an embodiment of an anti-CD180 antibody conjugated to the linker-payload deruxtecan (×8). FIG. 16B shows a DAR of 8 of an anti-CD180 antibody according to the present disclosure, CHAMPmAB-001, conjugated to the linker-payload deruxtecan (×8). FIG. 16C shows a DAR of 8.97 of an isotype IgG1 conjugated to the linker-payload deruxtecan (×8).

FIGS. 17A-17D show a therapeutic overview of the potent CD180-dependent cytotoxicity observed in primary tumors. FIG. 17A shows that tesirine conjugates are potent ADCs in CTG-2240 Primary AML: CO-ADC-001 is a tesirine (DAR 2) conjugated therapeutic anti-CD180 mAb. FIG. 17B shows that deruxtecan conjugates are also potent ADCs in CTG-2240 Primary AML and are comparable with MYLOTARG®. CO-ADC-004 is a deruxtecan (DAR 8) conjugated therapeutic anti-CD180 mAb. FIG. 17C shows that CO-ADC-001 cytotoxicity correlates with CD180 expression in Primary AML. FIG. 17D shows that Primary AML cytotoxicity results suggest a high frequency of responders.

FIGS. 18A-18C show that blocking CD180 with naked CD180 mAb reduces ADC activity. FIG. 18A shows the % viability of primary AML after addition of CO-ADC-001 (300 ng/mL) with no initial CD180 blocking Ab compared to the % viability of the primary AML after addition of CD180 ADCs when naked CD180 antibody 500 ng/mL was added prior to the addition of CD180 ADCs; the naked antibody blocked ADC binding to CD180 to CD180. FIG. 18B shows the % viability of primary AML after addition of CO-ADC-002 (300 ng/ml) with no initial CD180 blocking Ab compared to the % viability of the primary AML after addition of CD180 ADCs when naked CD180 antibody (500 ng/mL) was added prior to the addition of CD180 ADCs, which blocked ADC binding to CD180. FIG. 18C shows the % viability of primary AML after addition of isotype ADC (300 ng/mL) with no initial CD180 blocking Ab compared to the % viability of the primary AML after addition of isotype ADCs when naked CD180 antibody (500 ng/ml) was added prior to the addition of CD180 ADCs, which blocked the isotype ADC binding to CD180.

FIGS. 19A-19C show that CD180 ADCs are potent against Primary MCL. FIG. 19A shows the % viability of primary MCL with CO-ADC-001, a tesirine conjugated therapeutic anti-CD180 mAb, compared to the % viability of primary MCL with CO-ADC-003, a tesirine conjugated negative control mAb. FIG. 19A shows the % viability of Primary MCL CTG-3446 with CO-ADC-001 and CO-ADC-003, respectively. FIG. 19B shows the % viability of Primary MCL CTG-3785 with CO-ADC-001 and CO-ADC-003, respectively. FIG. 19C shows the % viability of Primary MCL CTG-3448 with CO-ADC-001 and CO-ADC-003, respectively.

FIGS. 20A-20B show that CO-ADC-001, a CD180-targeted DAR2 tesirine conjugate, is very active against disseminated primary AML in vivo. FIG. 20A shows the characteristics of CTG-2240 Primary AML. FIG. 20B shows results of in vivo analysis of % tumor in bone marrow, LSCs in bone marrow and CD123 cells in bone marrow after administration of 0.3 MPK of CO-ADC-001 compared to 0.3 MPK control and 0.3 MPK IgG-ADC.

FIGS. 21A-21C show that ADC CO-ADC-004, a CD180-targeted DAR8 deruxtecan conjugate, is very active against disseminated primary AML in vivo. FIG. 21A shows the characteristics of CTG-2240 Primary AML. FIG. 21B shows results of in vivo analysis of % hCD45 tumor in bone marrow, monocytes in bone marrow, CD123 cells in bone marrow, after administration of 5 MPK CO-ADC-004 compared to control (vehicle) and 5 MPK IgG-ADC (CO-ADC-005), as well as CD180+ LSCs in bone marrow, CD180+ monocytes in bone marrow, CD180+CD117+ in bone marrow, and CD180+CD123+ in bone marrow after administration of IgG-ADC or CO-ADC-004, an exemplary embodiment according to the present invention. FIG. 21C shows results of in vivo analysis of % CD180+ LSCs in bone marrow, % CD180+ monocytes in bone marrow, CD180+CD117+ in bone marrow and CD180+CD123+ in bone marrow.

FIGS. 22A-22G show the characteristics of one embodiment according to the present invention, anti-CD180 antibody lead CO-mAb-020 (CO-ADC-004), a fully human IgG1, i.e., melting temperature, self-interaction, freeze thaw stability, purity, poly reactivity, binding to CD180/MD1 HEK293 cells, and CO-mAb-020 internalization in MV-4-11. MV-4-11 is a human AML cell line established from blasts cells of 10 years old male with biphenotypic B-myelomonocytic leukemia (AML FAB M5) that carry translocation t(4;11) and a FLT3-ITD mutation.

FIGS. 23A-23H show conjugation of CO-mAB-20 to deruxtecan and tesirine. FIG. 23A shows one embodiment according to the present invention, anti-CD180 antibody lead CO-mAB-20 conjugated to deruxtecan (D×d)×8 with the chemical structure of D×d. FIG. 23B shows a DAR of 8 for the conjugate of antibody CO-mAB-20 to Deruxtecan (CO-ADC-004). FIG. 23C shows a DAR of 8.97 of an isotype IgG1 conjugated to the linker-payload deruxtecan (×8) (CO-ADC-005). FIG. 23D shows the antibody CO-mAB-20 conjugated to tesirine ×2. FIG. 23E shows a DAR of 2.1 for the conjugate of CO-mAB-20 to Tesirine ×2 (CO-ADC-001). FIG. 23F shows a DAR of 2.2 for the conjugate of an isotype IgG1 conjugated to the linker-payload tesirine ×2 (CO-ADC-003). FIGS. 23G-23H show that cellular binding to HEK293 CD180/MD-1 cells is unchanged after conjugation of CO-mAb-020 (CO-ADC-004) DAR:8 to deruxtecan (FIG. 23G) compared to conjugation of CO-mAb-020 (CO-ADC-001) DAR: 2.1 to tesirine (FIG. 23H). The ADCs were subjected to half maximal effective concentration (EC₅₀) assessment via cell-based ELISA using HEK293 cells expressing human CD180/MD-1.

FIGS. 24A-24B show that CO-ADC-004 lead ADC is potent against MV4-11 tumors in vivo. CO-ADC-004 is a CD180-D×d ADC. CO-ADC-005 is an Isotype-D×d ADC. MV4-11 Luc TVI injection was used for systemic engraftment. Mice were randomized when bioluminescence was detected above background in >90% of mice. A single dose of 5 mg/kg (MPK) was administered to each mouse by I.V. injection of the therapeutic, either 5 MPK CO-ADC-004 or 5 MPK CO-ADC-005. Tumor burden was assessed via bioluminescence. (FIG. 24B) CO-ADC-004 administered at a dose of 5 MPK showed complete regression for over 30 days compared to 5 MPK of the Isotype-D×d ADC (CO-ADC-005) (FIG. 24A).

FIGS. 25A-25B show that CO-ADC-001 ADC is potent against MV4-11 tumors in vivo.

CO-ADC-001 is a CD180-Tesirine ADC. CO-ADC-003 is an Isotype-Tesirine ADC. MV4-11 Luc TVI injection was used for systemic engraftment. Mice were randomized when bioluminescence was detected above background in >90% of mice. A single dose of 15 mg/kg (MPK) was administered to each mouse by I.V. injection of therapeutic, either CO-ADC-001 ADC or CO-ADC-003. Tumor burden was assessed via bioluminescence. (FIG. 25B) CO-ADC-001 ADC administered at a dose of 15 MPK showed complete regression for over 30 days compared to the Isotype-Tesirine ADC CO-ADC-003 (FIG. 25A).

FIGS. 26A-26C show that CD180 protein expression correlates with ADC cytotoxicity. FIGS. 26A-26B show that there is a wide range of sensitivity and cytotoxicity in AML patient samples. FIG. 26C shows CD180 protein expression and IC₅₀ correlation.

FIGS. 27A-27C show receptor expression correlation with ADC potency in other ADCs. FIG. 27A shows CD19 expression correlated with in vitro activity of Lonca Tesirine (loncastuximab tesirine-lpyl). FIG. 27B shows CD33 expression correlated with in vitro activity of IMGN779 (an anti-CD33 ADC with a DNA-alkylating IGN (indolinobenzodiazepine pseudodimer) payload and a cleavable s-SPDB linker). FIG. 27C shows CD123 expression correlated with in vitro activity of SGN-CD123A (an antibody-drug conjugate consisting of a humanized CD123 antibody conjugated to a pyrrolobenzodiazepine dimer (PBD) via a stable, protease-cleavable dipeptide linker with an engineered cysteine on each heavy chain attaching the PBD to the antibody for site-specific conjugation).

### DETAILED DESCRIPTION OF THE INVENTION

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number, respectively. Additionally, the words “herein,” “above,” and “below” and words of similar import, when used in this application, shall refer to this application as a whole and not to any particular portions of this application. As used herein, the singular forms “a”, “an” and “the” include plural referents unless the context clearly dictates otherwise. “And” as used herein is interchangeably used with “or” unless expressly stated otherwise.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of the invention and variations thereof are described. This description is not intended to be limiting and changes in the components, sequence of steps, and other variations would be understood to be within the scope of the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Throughout this application, various embodiments of the present disclosure may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When values are expressed as approximations, by use of the antecedent “about,” it is understood that the particular value forms another embodiment. All ranges are inclusive and combinable. In one embodiment, the term “about” refers to a deviance of between 0.1-5% from the indicated number or range of numbers. In another embodiment, the term “about” refers to a deviance of between 1-10% from the indicated number or range of numbers. In another embodiment, the term “about” refers to a deviance of up to 20% from the indicated number or range of numbers. In one embodiment, the term “about” refers to a deviance of ±10% from the indicated number or range of numbers. In another embodiment, the term “about” refers to a deviance of +5% from the indicated number or range of numbers.

In one embodiment, the present disclosure describes the isolation of a panel of anti-CD180 binding molecules and uses thereof. In one embodiment, the anti-CD180 binding molecule comprising a set of three heavy chain complementarity determining regions HCDR1, HCDR2 and HCDR3 as disclosed herein, and a corresponding set of three light chain complementarity determining regions LCDR1, LCDR2 and LCDR3 as disclosed herein.

In one embodiment, the anti-CD180 binding molecule comprises an anti-CD180 antibody. As used herein, the term “antibody” may be used interchangeably with the term “immunoglobulin”, having all the same qualities and meanings. An antibody binding domain or an antigen binding site can be a fragment of an antibody or a genetically engineered product of one or more fragments of the antibody, which fragment is involved in specifically binding with a target antigen. By “specifically binding” is meant that the binding is selective for the antigen of interest and can be discriminated from unwanted or nonspecific interactions. For example, an antibody is said to specifically bind a CD180 epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹²M. In some embodiments, the equilibrium dissociation constant may be in the range of ≤10⁻⁵ M to 10⁻¹²M.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

As used herein, the term “antibody” encompasses an antibody fragment or fragments that retain binding specificity including, but not limited to, IgG, heavy chain variable regions (VH), light chain variable regions (VL), Fab fragments, F(ab′)₂ fragments, scFv fragments, Fv fragments, a nanobody, minibodies, diabodies, triabodies, tetrabodies, and single domain antibodies (see, e.g., Hudson and Souriau, Nature Med. 9: 129-134 (2003)). Also encompassed are humanized, primatized, and chimeric antibodies as these terms are generally understood in the art.

As used herein, the term “heavy chain variable region” may be used interchangeably with the term “VH domain” or the term “VH”, having all the same meanings and qualities. As used herein, the term “light chain variable region” may be used interchangeably with the term “VL domain” or the term “VL”, having all the same meanings and qualities. A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions are more highly conserved than the CDRs and form a scaffold to support the CDRs. Similarly, a skilled artisan would also recognize that a “light chain variable region” or “VL” with regard to an antibody encompasses the fragment of the light chain that contains three CDRs interposed between framework regions.

As used herein, the term “complementarity determining region” or “CDR” refers to the hypervariable region(s) of a heavy or light chain variable region. Proceeding from the N-terminus, each heavy or light chain polypeptide has three CDRs denoted as “CDR1,” “CDR2,” and “CDR3”. Crystallographic analysis of a number of antigen-antibody complexes has demonstrated that the amino acid residues of CDRs form extensive contact with a bound antigen, wherein the most extensive antigen contact is with the heavy chain CDR3. Thus, the CDR regions are primarily responsible for the specificity of an antigen-binding site. In one embodiment, an antigen-binding site includes six CDRs, comprising the CDRs from each of a heavy and a light chain variable region.

A person of ordinary skill in the art would appreciate that a scFv is a fusion polypeptide comprising the variable heavy chain (VH) and variable light chain (VL) regions of an immunoglobulin, connected by a short linker peptide. The linker may have, for example, 10 to about 25 amino acids.

A skilled artisan would also appreciate that the term “Fab” with regard to an antibody generally encompasses that portion of the antibody consisting of a single light chain (both variable and constant regions) bound to the variable region and first constant region of a single heavy chain by a disulfide bond, whereas F(ab′)₂ comprises a fragment of a heavy chain comprising a VH domain and a light chain comprising a VL domain.

In some embodiments, an antibody encompasses whole antibody molecules, including monoclonal and polyclonal antibodies. In some embodiments, an antibody encompasses an antibody fragment or fragments that retain binding specificity including, but not limited to, variable heavy chain (VH) fragments, variable light chain (VL) fragments, Fab fragments, F(ab′)₂ fragments, scFv fragments, Fv fragments, minibodies, diabodies, triabodies, and tetrabodies.

In some embodiments, the anti-CD180 binding molecules of the present disclosure may be chimeric antibodies. A “chimeric antibody” is an immunoglobulin molecule in which the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.

In some embodiments, the anti-CD180 binding molecules of the present disclosure may be humanized antibodies. A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody). In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody can also comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

In some embodiments, the anti-CD180 binding molecules of the present disclosure are bispecific (or multi-specific) antibodies. As it is generally known in the art, a bispecific antibody is a recombinant protein that includes antigen-binding fragments of two different monoclonal antibodies, and is thereby capable of binding two different antigens. In some embodiments, the bispecific antibodies are monoclonal, preferably human or humanized, antibodies that have binding specificities for at least one other antigen besides CD180 or that have binding specificities for different CD180 epitopes. Similarly, a multi-specific antibody is a recombinant protein that includes antigen-binding fragments of at least two different monoclonal antibodies, such as two, three or four different monoclonal antibodies. In an embodiment, the bispecific anti-CD180 antibody is a bi-paratopic antibody, i.e., a bispecific antibody that targets two non-overlapping epitopes on the same target antigen. In certain embodiments, the bispecific antibody binds to a CD180 and a CD123 (“a CD180×CD123 bi-specific antibody”). In some embodiments, the bispecific anti-CD180 antibody comprises the variable heavy chain (VH) and variable light chain (VL) regions and/or the CDRs disclosed herein and the VH, VL, and/or the CDRs combinations disclosed herein that have binding specificity to CD180, i.e., comprises a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each comprise the amino acid sequences disclosed herein.

In another embodiment, one of ordinary skill in the art would readily use the VH, VL, and/or the CDRs disclosed herein to construct chimeric antigen receptor (CAR) that would have binding specificity to CD180.

In one embodiment, the anti-CD180 antibody or an antigen binding fragment thereof may comprise one or more Fc domain mutations that impair binding to the FcγR receptor (e.g. FcγI, FcγIIa, FcγIIb, or FcγRIIa). Any suitable Fc domain mutants can be used so that the resulting Fc domain binding to the FcγR receptor is reduced, e.g. by at least 50% relative to that with a non-mutated Fc domain. Fc mutations and truncations that can be made to reduce binding to the FcγR receptor can be made by those of skill in the art based on techniques well-known in the art.

In some embodiments, the anti-CD180 binding molecules or anti-CD180 antibodies of the present disclosure can further be conjugated to an effector moiety. The effector moiety can be any number of molecules, including labeling moieties such as radioactive labels or fluorescent labels, or a therapeutic moiety. If the effector moiety is a therapeutic moiety, it will typically be a cytotoxic agent. Cytotoxic agents are numerous and varied and include, but are not limited to, cytotoxic drugs or toxins or active fragments of such toxins. Suitable toxins and their corresponding fragments are well-known in the art. Cytotoxic agents also include radiochemicals made by conjugating radioisotopes to antibodies, or binding of a radionuclide to a chelating agent that has been attached to the antibody.

In another embodiment, the anti-CD180 binding molecules or anti-CD180 antibodies of the present disclosure may be modified to extend half-life, such as by attaching at least one molecule to the antibody for extending serum half-life, including but not limited to a polyethylene glycol (PEG) group, serum albumin, transferrin, transferrin receptor or the transferrin-binding portion thereof, or combinations thereof. As used herein, the word “attached” refers to a covalently or noncovalently conjugated substance. The conjugation may be by genetic engineering or by chemical means.

### Anti-CD180 Binding Molecules

The present disclosure provides a number of anti-CD180 binding molecule comprising a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 comprises the amino acid sequences as shown in Table 1, and the set of corresponding LCDR1, LCDR2 and LCDR3 comprises the amino acid sequences as shown in Table 2. For the purpose of illustration, take Ab-001 as an example (see Table 1), the set of HCDR1, HCDR2, and HCDR3 comprises the amino acid sequences of SEQ ID NOs:1-3 respectively (see Table 1), whereas the corresponding set of LCDR1, LCDR2, and LCDR3 comprises the amino acid sequences of SEQ ID NOs:4-6 respectively (see Table 2).

In one embodiment, the set of HCDR1, HCDR2, and HCDR3 comprises the amino acid sequences set forth in SEQ ID NOs:1-3 respectively, and the corresponding set of LCDR1, LCDR2, and LCDR3 comprises the amino acid sequences set forth in SEQ ID NOs:4, 5, 15 respectively. In one embodiment, the set of HCDR1, HCDR2, and HCDR3 comprises the amino acid sequences set forth in SEQ ID NOs:1, 24, 25 respectively, and the corresponding set of LCDR1, LCDR2, and LCDR3 comprises the amino acid sequences set forth in SEQ ID NOs:4, 5, 26 respectively. In one embodiment, the set of HCDR1, HCDR2, and HCDR3 comprises the amino acid sequences set forth in SEQ ID NOs:1, 35, 25 respectively, and the corresponding set of LCDR1, LCDR2, and LCDR3 comprises the amino acid sequences set forth in SEQ ID NOs:4, 5, 26 respectively. In one embodiment, the set of HCDR1, HCDR2, and HCDR3 comprises the amino acid sequences set forth in SEQ ID NOs:1, 44, 25 respectively, and the corresponding set of LCDR1, LCDR2, and LCDR3 comprises the amino acid sequences set forth in SEQ ID NOs:4, 5, 45 respectively. In one embodiment, the set of HCDR1, HCDR2, and HCDR3 comprises the amino acid sequences set forth in SEQ ID NOs:54-56 respectively, and the corresponding set of LCDR1, LCDR2, and LCDR3 comprises the amino acid sequences set forth in SEQ ID NOs:57-59 respectively. In one embodiment, the set of HCDR1, HCDR2, and HCDR3 comprises the amino acid sequences set forth in SEQ ID NOs:68-70 respectively, and the corresponding set of LCDR1, LCDR2, and LCDR3 comprises the amino acid sequences set forth in SEQ ID NOs:57, 58, 71 respectively. In one embodiment, the set of HCDR1, HCDR2, and HCDR3 comprises the amino acid sequences set forth in SEQ ID NOs:1, 24, 25 respectively, and the corresponding set of LCDR1, LCDR2, and LCDR3 comprises the amino acid sequences set forth in SEQ ID NOs:80-82 respectively. In one embodiment, the set of HCDR1, HCDR2, and HCDR3 comprises the amino acid sequences set forth in SEQ ID NOs:1, 35, 25 respectively, and the corresponding set of LCDR1, LCDR2, and LCDR3 comprises the amino acid sequences set forth in SEQ ID NOs:91-93 respectively. In one embodiment, the set of HCDR1, HCDR2, and HCDR3 comprises the amino acid sequences set forth in SEQ ID NOs:1, 102, 25 respectively, and the corresponding set of LCDR1, LCDR2, and LCDR3 comprises the amino acid sequences set forth in SEQ ID NOs:4, 103, 6 respectively. In one embodiment, the set of HCDR1, HCDR2, and HCDR3 comprises the amino acid sequences set forth in SEQ ID NOs:1, 24, 25 respectively, and the corresponding set of LCDR1, LCDR2, and LCDR3 comprises the amino acid sequences set forth in SEQ ID NOs:112, 5, 113 respectively. In one embodiment, the set of HCDR1, HCDR2, and HCDR3 comprises the amino acid sequences set forth in SEQ ID NOs:1, 35, 25 respectively, and the corresponding set of LCDR1, LCDR2, and LCDR3 comprises the amino acid sequences set forth in SEQ ID NOs:4, 5, 6 respectively. In one embodiment, the set of HCDR1, HCDR2, and HCDR3 comprises the amino acid sequences set forth in SEQ ID NOs:1, 44, 25 respectively, and the corresponding set of LCDR1, LCDR2, and LCDR3 comprises the amino acid sequences set forth in SEQ ID NOs:4, 5, 130 respectively. In one embodiment, the set of HCDR1, HCDR2, and HCDR3 comprises the amino acid sequences set forth in SEQ ID NOs:1, 24, 25 respectively, and the corresponding set of LCDR1, LCDR2, and LCDR3 comprises the amino acid sequences set forth in SEQ ID NOs:4, 5, 6 respectively.

In another embodiment, the anti-CD180 binding molecule disclosed herein comprises heavy chain and light chain CDR sequences that are at least 80% (e.g., at least 85%, 90%, 95%, 96%, 97%, 98%, or 99%) identical to the amino acid sequences set forth above, for example but not limited to identity as determined using BlastP software of the National Center of Biotechnology Information (NCBI) using default parameters.

When used in relation to polypeptide (or nucleotide) sequences, the term “identity” refers to the degree of identity between two or more polypeptide (or nucleotide) sequences or fragments thereof. Typically, the degree of similarity between two or more polypeptide (or nucleotide) sequences refers to the degree of similarity of the composition, order, or arrangement of two or more amino acids or nucleotides of the two or more polypeptide (or nucleotide) sequences. A skilled artisan would appreciate that percent identity (% identity) provides a number that describes how similar the query sequence is to the target sequence. The higher the percent identity is, the more significant the match.

In another embodiment, the anti-CD180 binding molecule disclosed herein comprises a heavy chain variable region and a light chain variable region, wherein the heavy chain variable region and light chain variable region comprise the amino acid sequences of SEQ ID NOs:7-8, SEQ ID NOs:16-17, SEQ ID NOs:27-28, SEQ ID NOs:36-37, SEQ ID NOs:46-47, SEQ ID NOs:60-61, SEQ ID NOs:72-73, SEQ ID NOs:83-84, SEQ ID NOs:94-95, SEQ ID NOs:104-105, SEQ ID NOs:114-115, SEQ ID NOs:122-123, SEQ ID NOs:131-132, or SEQ ID NOs:139-140.

In another embodiment, the anti-CD180 binding molecule disclosed herein comprises a heavy chain variable region and a light chain variable region that are at least 80% (e.g., at least 85%, 90%, 95%, 96%, 97%, 98%, or 99%) identical to the amino acid sequences set forth above, for example but not limited to identity as determined using BlastP software of the National Center of Biotechnology Information (NCBI) using default parameters.

In another embodiment, the anti-CD180 binding molecule disclosed herein comprises a heavy chain and a light chain, wherein the heavy chain and light chain comprise the amino acid sequences of SEQ ID NOs: 11-12, SEQ ID NOs:20-21, SEQ ID NOs:31-32, SEQ ID NOs:40-41, SEQ ID NOs:50-51, SEQ ID NOs:64-65, SEQ ID NOs:76-77, SEQ ID NOs:87-88, SEQ ID NOs:98-99, SEQ ID NOs:108-109, SEQ ID NOs:118-119, SEQ ID NOs:126-127, SEQ ID NOs:135-136, or SEQ ID NOs:143-144.

In another embodiment, the anti-CD180 binding molecule disclosed herein comprises a heavy chain and a light chain that are at least 80% (e.g., at least 85%, 90%, 95%, 96%, 97%, 98%, or 99%) identical to the amino acid sequences set forth above, for example but not limited to identity as determined using BlastP software of the National Center of Biotechnology Information (NCBI) using default parameters.

In another embodiment, there are provided isolated polynucleotide sequences encoding the anti-CD180 binding molecules disclosed herein. In one embodiment, the anti-CD180 binding molecules comprise a heavy chain variable region encoded by nucleotide sequence having the sequence of SEQ ID NO:9, 18, 29, 38, 48, 62, 74, 85, 96, 106, 116, 124, 133 or 141. In one embodiment, the anti-CD180 binding molecules comprise a light chain variable region encoded by nucleotide sequence having the sequence of SEQ ID NO:10, 19, 30, 39, 49, 63, 75, 86, 97, 107, 117, 125, 134 or 142. In one embodiment, the anti-CD180 binding molecules comprise a heavy chain encoded by nucleotide sequence having the sequence of SEQ ID NO:13, 22, 33, 42, 52, 66, 78, 89, 100, 110, 120, 128, 137 or 145. In one embodiment, the anti-CD180 binding molecules comprise a light chain encoded by nucleotide sequence having the sequence of SEQ ID NO:14, 23, 34, 43, 53, 67, 79, 90, 101, 111, 121, 129, 138 or 146.

In another embodiment, the present disclosure encompasses polynucleotide sequences that are at least 80% (e.g., at least 85%, 90%, 95%, 96%, 97%, 98%, or 99%) identical to the polynucleotide sequences set forth above, as determined by methods and parameters generally known in the art.

### Compositions of Use

In one embodiment, the present disclosure also provides a composition comprising the anti-CD180 binding molecules disclosed herein and a pharmaceutically acceptable carrier. Pharmaceutically acceptable carriers of use are well-known in the art. For example, Remington's Pharmaceutical Sciences, by E. W. Martin, Mack Publishing Co., Easton, PA, 23rd Edition, 2020 describes compositions and formulations suitable for pharmaceutical delivery of the polypeptides or antibodies disclosed herein. In one embodiment, the composition comprises antibody drug conjugates as disclosed herein. In one embodiment, the antibody drug conjugates comprise tesirine. In some embodiments, the ADCs comprise deruxtecan.

One of ordinary skill in the art would readily incorporate the anti-CD180 binding molecules disclosed herein into therapeutics that target cells expressing CD180. Examples of such therapeutic modalities include, but are not limited to, monoclonal antibodies, antibody drug conjugates, chimeric antigen receptor T-cells, and chimeric antigen receptor natural killer cells. In one embodiment, the therapeutics can be used to stimulate or activate cells expressing CD180.

A composition comprising an anti-CD180 binding molecule or an antigen-binding fragment thereof as disclosed herein can be administered to a subject (e.g., a human or an animal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. The pharmaceutical compositions may be prepared by methodologies well known in the pharmaceutical art.

In one embodiment, the composition comprises an anti-CD180 binding molecule that comprises a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 comprises the amino acid sequences as shown in Table 1, and the set of corresponding LCDR1, LCDR2 and LCDR3 comprises the amino acid sequences as shown in Table 2.

In another embodiment, the present disclosure also provides a composition comprising any of the polynucleotide sequences disclosed herein, wherein the polynucleotide sequences encode the anti-CD180 binding molecules disclosed herein.

The compositions comprising the anti-CD180 binding molecules or antigen-binding fragments thereof disclosed herein, or the polynucleotide sequences disclosed herein that encode the anti-CD180 binding molecules, can be administered (e.g., to a mammal, a cell, or a tissue) in any suitable manner depending on whether local or systemic treatment is desired. For example, the composition can be administered topically (e.g., ophthalmically, vaginally, rectally, intranasally, transdermally, and the like), orally, by inhalation, or parenterally (including by intravenous drip or subcutaneous, intracavity, intraperitoneal, intradermal, or intramuscular injection). Topical intranasal administration refers to delivery of the compositions into the nose and nasal passages through one or both of the nares. The composition can be delivered by a spraying mechanism or droplet mechanism, or through aerosolization. Alternatively, administration can be intratumoral, e.g., local or intravenous injection.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve preparation of a slow-release or sustained-release system so as to maintain a constant dosage.

The present disclosure provides an anti-CD180 antibody-drug conjugate (ADC) comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the anti-CD180 antibody or antigen-binding fragment thereof comprises a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each comprise the amino acid sequences:
HCDR1, HCDR2 and HCDR3 of SEQ ID NOs: 1-3 and LCDR1, LCDR2 and LCDR3 of SEQ ID NOs: 4-6;
HCDR1, HCDR2 and HCDR3 of SEQ ID NOs: 1-3 and LCDR1, LCDR2 and LCDR3 of SEQ ID NOs: 4, 5, and 15;
HCDR1, HCDR2 and HCDR3 of SEQ ID NOs: 1, 24, and 25 and LCDR1, LCDR2 and LCDR3 of SEQ ID NOs: 4, 5, and 26;
HCDR1, HCDR2 and HCDR3 of SEQ ID NOs: 1, 35, and 25 and LCDR1, LCDR2 and LCDR3 of SEQ ID NOs: 4, 5, and 26;
HCDR1, HCDR2 and HCDR3 of SEQ ID NOs: 1, 44, and 25 and LCDR1, LCDR2 and LCDR3 of SEQ ID NOs: 4, 5, and 45;
HCDR1, HCDR2 and HCDR3 of SEQ ID NOs: 54-56 and LCDR1, LCDR2 and LCDR3 of SEQ ID NOs: 57-59;
HCDR1, HCDR2 and HCDR3 of SEQ ID NOs: 68-70 and LCDR1, LCDR2 and LCDR3 of SEQ ID NOs: 57, 58, and 71;
HCDR1, HCDR2 and HCDR3 of SEQ ID NOs: 1, 24, and 25 and LCDR1, LCDR2 and LCDR3 of SEQ ID NOs: 80-82;
HCDR1, HCDR2 and HCDR3 of SEQ ID NOs: 1, 35, and 25 and LCDR1, LCDR2 and LCDR3 of SEQ ID NOs: 91-93;
HCDR1, HCDR2 and HCDR3 of SEQ ID NOs: 1, 102, and 25 and LCDR1, LCDR2 and LCDR3 of SEQ ID NOs: 4, 103, and 6;
HCDR1, HCDR2 and HCDR3 of SEQ ID NOs: 1, 24, and 25 and LCDR1, LCDR2 and LCDR3 of SEQ ID NOs: 112, 5, and 113;
HCDR1, HCDR2 and HCDR3 of SEQ ID NOs: 1, 35, and 25 and LCDR1, LCDR2 and LCDR3 of SEQ ID NOs: 4-6;
HCDR1, HCDR2 and HCDR3 of SEQ ID NOs: 1, 44, and 25 and LCDR1, LCDR2 and LCDR3 of SEQ ID NOs: 4, 5, and 130; or
HCDR1, HCDR2 and HCDR3 of SEQ ID NOs: 1, 24, and 25 and LCDR1, LCDR2 and LCDR3 of SEQ ID NOs: 4-6.

The present disclosure provides an anti-CD180 antibody-drug conjugate (ADC) comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the anti-CD180 antibody or antigen-binding fragment thereof comprises a heavy chain variable region and a light chain variable region, said heavy chain variable region and light chain variable region comprise the amino acid sequences of SEQ ID NOs:7-8, SEQ ID NOs:16-17, SEQ ID NOs:27-28, SEQ ID NOs:36-37, SEQ ID NOs:46-47, SEQ ID NOs:60-61, SEQ ID NOs:72-73, SEQ ID NOs:83-84, SEQ ID NOs:94-95, SEQ ID NOs:104-105, SEQ ID NOs:114-115, SEQ ID NOs:122-123, SEQ ID NOs:131-132, or SEQ ID NOs:139-140.

The present disclosure provides an anti-CD180 antibody-drug conjugate (ADC) comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the anti-CD180 antibody or antigen-binding fragment thereof comprises a heavy chain and a light chain, said heavy chain and light chain comprise the amino acid sequences of SEQ ID NOs: 11-12, SEQ ID NOs:20-21, SEQ ID NOs:31-32, SEQ ID NOs:40-41, SEQ ID NOs:50-51, SEQ ID NOs:64-65, SEQ ID NOs:76-77, SEQ ID NOs:87-88, SEQ ID NOs:98-99, SEQ ID NOs:108-109, SEQ ID NOs:118-119, SEQ ID NOs:126-127, SEQ ID NOs:135-136, or SEQ ID NOs:143-144.

In an embodiment of the herein provided ADC, the anti-CD180 antibody or antigen-binding fragment thereof comprises an IgG, a Fv, a scFv, a Fab, a F(ab′)₂, a minibody, a diabody, a triabody, a nanobody, a bispecific antibody, a single domain antibody, or a chimeric antigen receptor. In an embodiment, the bispecific anti-CD180 antibody is a bi-paratopic antibody, i.e., a bispecific antibody that targets two non-overlapping epitopes on the same target antigen. In a particular embodiment, the bispecific antibody binds to a CD180 and a CD123 (“a CD180×CD123 bi-specific antibody”). In some embodiments, the bispecific anti-CD180 antibody comprises the variable heavy chain (VH) and variable light chain (VL) regions and/or the CDRs disclosed herein and the VH, VL, and/or the CDRs combinations disclosed herein that have binding specificity to CD180, i.e., comprises a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each comprise the amino acid sequences disclosed herein.

In some embodiments of the ADC, the IgG is IgG1, IgG2, IgG3, or IgG4. In a particular embodiment of the ADCs, the cytotoxic drug payload comprises a ADC drug-linker conjugate tesirine (SG3249), the tesirine comprising a cytotoxic drug payload, the cytotoxic drug payload comprising pyrrolobenzodiazepine dimer cytotoxic DNA-alkylating agent (SG3199), wherein the tesirine is conjugated to the CD180-high expressing tumor-targeting monoclonal antibody or antigen-binding fragment thereof via a cleavable linker moiety.

In some embodiment of the ADCs, the ADC comprises an ADC drug-linker conjugate deruxtecan, the deruxtecan comprising a cleavable linker, a self-immolative amino methylene spacer, and a cytotoxic drug payload, the cytotoxic drug payload comprising a topoisomerase 1 inhibitor payload, wherein the topoisomerase 1 inhibitor payload is a derivative of exatecan (DX-8951), wherein the deruxetan is conjugated to the CD180-high expressing tumor-targeting monoclonal antibody or antigen-binding fragment thereof via the cleavable linker, wherein the cleavable linker is a glycine-glycine-phenylalanine-glycine tetrapeptide-based linker.

In certain embodiments, the cleavable linker moiety is a hydrazone linker, a disulphide linker or a peptide linker. In various embodiments of the ADCs, the peptide linker is a dipeptide linker selected from the group consisting of valine-citrulline (Val-Cit), valine-alanine (Val-Ala) and alanine-alanine (Ala-Ala). In an embodiment, the dipeptide linker is joined to the cytotoxic drug payload by a spacer unit, wherein the spacer unit is para-aminobenzyloxycarbonyl (PABC). In some embodiments of the herein provided ADCs, the peptide linker is a tripeptide linker, wherein the tripeptide linker is a glutamic acid-valine-citrulline (EVCit) tripeptide linker. In an embodiment, the glutamic acid-valine-citrulline (EVCit) tripeptide linker is joined to a meta-amide para-aminobenzyl carbamate (MA-PABC) group. In a particular embodiment of the described and provided ADCs comprising the ADC drug-linker conjugate tesirine (SG3249), the drug-to-antibody ratio (DAR) is 2.1. In some embodiments of the ADCs comprising the ADC drug-linker conjugate deruxtecan, the drug-to-antibody ratio (DAR) is 8.

The present disclosure provides a method of treating a cancer in a subject, the method comprising the step of administering to the subject an ADC comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the CD180-high expressing tumor-targeting monoclonal antibody or antigen-binding fragment thereof is anti-CD180 antibody or antigen-binding fragment thereof comprising a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each comprise the amino acid sequences:
HCDR1, HCDR2 and HCDR3 of SEQ ID NOs: 1-3 and LCDR1, LCDR2 and LCDR3 of SEQ ID NOs: 4-6;
HCDR1, HCDR2 and HCDR3 of SEQ ID NOs: 1-3 and LCDR1, LCDR2 and LCDR3 of SEQ ID NOs: 4, 5, and 15;
HCDR1, HCDR2 and HCDR3 of SEQ ID NOs: 1, 24, and 25 and LCDR1, LCDR2 and LCDR3 of SEQ ID NOs: 4, 5, and 26;
HCDR1, HCDR2 and HCDR3 of SEQ ID NOs: 1, 35, and 25 and LCDR1, LCDR2 and LCDR3 of SEQ ID NOs: 4, 5, and 26;
HCDR1, HCDR2 and HCDR3 of SEQ ID NOs: 1, 44, and 25 and LCDR1, LCDR2 and LCDR3 of SEQ ID NOs: 4, 5, and 45;
HCDR1, HCDR2 and HCDR3 of SEQ ID NOs: 54-56 and LCDR1, LCDR2 and LCDR3 of SEQ ID NOs: 57-59;
HCDR1, HCDR2 and HCDR3 of SEQ ID NOs: 68-70 and LCDR1, LCDR2 and LCDR3 of SEQ ID NOs: 57, 58, and 71;
HCDR1, HCDR2 and HCDR3 of SEQ ID NOs: 1, 24, and 25 and LCDR1, LCDR2 and LCDR3 of SEQ ID NOs: 80-82;
HCDR1, HCDR2 and HCDR3 of SEQ ID NOs: 1, 35, and 25 and LCDR1, LCDR2 and LCDR3 of SEQ ID NOs: 91-93;
HCDR1, HCDR2 and HCDR3 of SEQ ID NOs: 1, 102, and 25 and LCDR1, LCDR2 and LCDR3 of SEQ ID NOs: 4, 103, and 6;
HCDR1, HCDR2 and HCDR3 of SEQ ID NOs: 1, 24, and 25 and LCDR1, LCDR2 and LCDR3 of SEQ ID NOs: 112, 5, and 113;
HCDR1, HCDR2 and HCDR3 of SEQ ID NOs: 1, 35, and 25 and LCDR1, LCDR2 and LCDR3 of SEQ ID NOs: 4-6;
HCDR1, HCDR2 and HCDR3 of SEQ ID NOs: 1, 44, and 25 and LCDR1, LCDR2 and LCDR3 of SEQ ID NOs: 4, 5, and 130; or
HCDR1, HCDR2 and HCDR3 of SEQ ID NOs: 1, 24, and 25 and LCDR1, LCDR2 and LCDR3 of SEQ ID NOs: 4-6.

The present disclosure provides a method of treating a cancer in a subject, the method comprising the step of administering to the subject an ADC comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the CD180-high expressing tumor-targeting monoclonal antibody or antigen-binding fragment thereof is an anti-CD180 antibody or antigen-binding fragment thereof comprising a heavy chain variable region and a light chain variable region, said heavy chain variable region and light chain variable region comprise the amino acid sequences of SEQ ID NOs:7-8, SEQ ID NOs:16-17, SEQ ID NOs:27-28, SEQ ID NOs:36-37, SEQ ID NOs:46-47, SEQ ID NOs:60-61, SEQ ID NOs:72-73, SEQ ID NOs:83-84, SEQ ID NOs:94-95, SEQ ID NOs:104-105, SEQ ID NOs:114-115, SEQ ID NOs:122-123, SEQ ID NOs:131-132, or SEQ ID NOs:139-140.

The present disclosure provides a method of treating a cancer in a subject, the method comprising the step of administering to the subject an ADC comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the CD180-high expressing tumor-targeting monoclonal antibody or antigen-binding fragment thereof is an anti-CD180 antibody or antigen-binding fragment thereof comprising a heavy chain and a light chain, said heavy chain and light chain comprise the amino acid sequences of SEQ ID NOs: 11-12, SEQ ID NOs:20-21, SEQ ID NOs:31-32, SEQ ID NOs:40-41, SEQ ID NOs:50-51, SEQ ID NOs:64-65, SEQ ID NOs:76-77, SEQ ID NOs:87-88, SEQ ID NOs:98-99, SEQ ID NOs:108-109, SEQ ID NOs:118-119, SEQ ID NOs:126-127, SEQ ID NOs:135-136, or SEQ ID NOs:143-144.

The present disclosure provides a method of treating a cancer in a subject, the method comprising the step of administering to the subject an ADC comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the cytotoxic drug payload comprises a ADC drug-linker conjugate tesirine (SG3249), the tesirine comprising a cytotoxic drug payload, the cytotoxic drug payload comprising pyrrolobenzodiazepine dimer cytotoxic DNA-alkylating agent (SG3199), wherein the tesirine is conjugated to the CD180-high expressing tumor-targeting monoclonal antibody or antigen-binding fragment thereof via a cleavable linker moiety. wherein the CD180-high expressing tumor-targeting monoclonal antibody or antigen-binding fragment thereof is an anti-CD180 antibody or antigen-binding fragment thereof comprising a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each comprise the amino acid sequences:
HCDR1, HCDR2 and HCDR3 of SEQ ID NOs: 1-3 and LCDR1, LCDR2 and LCDR3 of SEQ ID NOs: 4-6;
HCDR1, HCDR2 and HCDR3 of SEQ ID NOs: 1-3 and LCDR1, LCDR2 and LCDR3 of SEQ ID NOs: 4, 5, and 15;
HCDR1, HCDR2 and HCDR3 of SEQ ID NOs: 1, 24, and 25 and LCDR1, LCDR2 and LCDR3 of SEQ ID NOs: 4, 5, and 26;
HCDR1, HCDR2 and HCDR3 of SEQ ID NOs: 1, 35, and 25 and LCDR1, LCDR2 and LCDR3 of SEQ ID NOs: 4, 5, and 26;
HCDR1, HCDR2 and HCDR3 of SEQ ID NOs: 1, 44, and 25 and LCDR1, LCDR2 and LCDR3 of SEQ ID NOs: 4, 5, and 45;
HCDR1, HCDR2 and HCDR3 of SEQ ID NOs: 54-56 and LCDR1, LCDR2 and LCDR3 of SEQ ID NOs: 57-59;
HCDR1, HCDR2 and HCDR3 of SEQ ID NOs: 68-70 and LCDR1, LCDR2 and LCDR3 of SEQ ID NOs: 57, 58, and 71;
HCDR1, HCDR2 and HCDR3 of SEQ ID NOs: 1, 24, and 25 and LCDR1, LCDR2 and LCDR3 of SEQ ID NOs: 80-82;
HCDR1, HCDR2 and HCDR3 of SEQ ID NOs: 1, 35, and 25 and LCDR1, LCDR2 and LCDR3 of SEQ ID NOs: 91-93;
HCDR1, HCDR2 and HCDR3 of SEQ ID NOs: 1, 102, and 25 and LCDR1, LCDR2 and LCDR3 of SEQ ID NOs: 4, 103, and 6;
HCDR1, HCDR2 and HCDR3 of SEQ ID NOs: 1, 24, and 25 and LCDR1, LCDR2 and LCDR3 of SEQ ID NOs: 112, 5, and 113;
HCDR1, HCDR2 and HCDR3 of SEQ ID NOs: 1, 35, and 25 and LCDR1, LCDR2 and LCDR3 of SEQ ID NOs: 4-6;
HCDR1, HCDR2 and HCDR3 of SEQ ID NOs: 1, 44, and 25 and LCDR1, LCDR2 and LCDR3 of SEQ ID NOs: 4, 5, and 130; or
HCDR1, HCDR2 and HCDR3 of SEQ ID NOs: 1, 24, and 25 and LCDR1, LCDR2 and LCDR3 of SEQ ID NOs: 4-6.

The present disclosure provides a method of treating a cancer in a subject, the method comprising the step of administering to the subject an ADC comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the ADC comprises an ADC drug-linker conjugate deruxtecan, the deruxetan comprising a cleavable linker, a self-immolative amino methylene spacer, and a cytotoxic drug payload, the cytotoxic drug payload comprising a topoisomerase 1 inhibitor payload, wherein the topoisomerase 1 inhibitor payload is a derivative of exatecan (DX-8951), wherein the deruxetan is conjugated to the CD180-high expressing tumor-targeting monoclonal antibody or antigen-binding fragment thereof via the cleavable linker, wherein the cleavable linker is a glycine-glycine-phenylalanine-glycine tetrapeptide-based linker.

In some embodiments, the cancer is acute myeloid leukemia (AML), mantle cell lymphoma (MCL), multiple myeloma, follicular lymphoma, B-acute lymphoblastic leukemia, or diffuse large B-cell lymphoma. In particular embodiments, the AML is primary AML or primary MCL. In an embodiment, the ADC comprises the set of HCDR1, HCDR2 and HCDR3 comprising the amino acids of SEQ ID NOs: 1-3 and the set of corresponding LCDR1, LCDR2 and LCDR3 comprising the amino acid sequences of SEQ ID NOs: 4-6, wherein the cytotoxic drug payload comprises a pyrrolobenzodiazepine dimer cytotoxic alkylating agent tesirine (SG3199) conjugated to the CD180-high expressing tumor-targeting monoclonal antibody or antigen-binding fragment thereof via a cleavable linker moiety, wherein the cleavable linker moiety is a valine-alanine (Val-Ala) peptide linker. In a particular embodiment, the Val-Ala peptide linker is cathepsin B-cleavable. In a particular embodiment of the described and provided ADCs comprising the ADC drug-linker conjugate tesirine (SG3249), the drug-to-antibody ratio (DAR) is 2.1

In an embodiment of the herein provided therapeutic methods, the cancer is primary AML. In some embodiment of the provided methods, the cancer is primary MCL. In a particular embodiment, the ADC comprises the set of HCDR1, HCDR2 and HCDR3 comprising the amino acids of SEQ ID NOs: 1, 35 and 25 and the set of corresponding LCDR1, LCDR2 and LCDR3 comprising the amino acid sequences of SEQ ID NOs: 4, 5 and 26, wherein the ADC is conjugated to an ADC drug-linker conjugate deruxtecan, the deruxtecan comprising a cleavable glycine-glycine-phenylalanine-glycine tetrapeptide-based linker, a self-immolative amino methylene spacer, and a cytotoxic drug payload, the cytotoxic drug payload comprising a topoisomerase 1 inhibitor payload, wherein the topoisomerase 1 inhibitor payload is a derivative of exatecan (DX-8951), wherein the deruxtecan is conjugated to the CD180-high expressing tumor-targeting monoclonal antibody or antigen-binding fragment thereof via the cleavable glycine-glycine-phenylalanine-glycine tetrapeptide-based linker. In some embodiments of the ADCs comprising the ADC drug-linker conjugate deruxtecan, the drug-to-antibody ratio (DAR) is 8. In certain embodiments of the provided therapeutic methods, the cancer is primary AML. In some embodiments, the cancer is primary MCL.

The present disclosure provides a method of modulating CD180-mediated immune responses in a subject, comprising administering to the subject any one of herein described and provided ADCs. In an embodiment, the CD180-mediated immune responses are antigen presenting cell activities of B cells, antibody secretion, or proliferation of B lymphocytes, monocytes or dendritic cells. In some embodiments, modulation of CD180-mediated immune responses comprises enhancing said CD180-mediated immune responses or inhibiting said CD180-mediated immune responses.

The present disclosure provides a method of treating a disease in a subject, comprising the step of administering to the subject any of the ADCs provided herein In some embodiments, the disease is a viral infection, a bacterial infection, an autoimmune disease or an immune disorder.

The present disclosure provides an isolated anti-CD180 binding molecule comprising a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each comprise the amino acid sequences:
HCDR1, HCDR2 and HCDR3 of SEQ ID NOs: 1-3 and LCDR1, LCDR2 and LCDR3 of SEQ ID NOs: 4-6;
HCDR1, HCDR2 and HCDR3 of SEQ ID NOs: 1-3 and SEQ ID NOs: 4, 5, and 15;
HCDR1, HCDR2 and HCDR3 of SEQ ID NOs: 1, 24, and 25 and SEQ ID NOs: 4, 5, and 26;
HCDR1, HCDR2 and HCDR3 of SEQ ID NOs: 1, 35, and 25 and SEQ ID NOs: 4, 5, and 26;
HCDR1, HCDR2 and HCDR3 of SEQ ID NOs: 1, 44, and 25 and SEQ ID NOs: 4, 5, and 45;
HCDR1, HCDR2 and HCDR3 of SEQ ID NOs: 54-56 and SEQ ID NOs: 57-59;
HCDR1, HCDR2 and HCDR3 of SEQ ID NOs: 68-70 and SEQ ID NOs: 57, 58, and 71;
HCDR1, HCDR2 and HCDR3 of SEQ ID NOs: 1, 24, and 25 and SEQ ID NOs: 80-82;
HCDR1, HCDR2 and HCDR3 of SEQ ID NOs: 1, 35, and 25 and SEQ ID NOs: 91-93;
HCDR1, HCDR2 and HCDR3 of SEQ ID NOs: 1, 102, and 25 and SEQ ID NOs: 4, 103, and 6;
HCDR1, HCDR2 and HCDR3 of SEQ ID NOs: 1, 24, and 25 and SEQ ID NOs: 112, 5, and 113;
HCDR1, HCDR2 and HCDR3 of SEQ ID NOs: 1, 35, and 25 and SEQ ID NOs: 4-6;
HCDR1, HCDR2 and HCDR3 of SEQ ID NOs: 1, 44, and 25 and SEQ ID NOs: 4, 5, and 130;
HCDR1, HCDR2 and HCDR3 of SEQ ID NOs: 1, 24, and 25 and SEQ ID NOs: 4-6.

In an embodiment of the ADCs, the anti-CD180 binding molecule comprises a heavy chain variable region and a light chain variable region, said heavy chain variable region and light chain variable region comprise the amino acid sequences of SEQ ID NOs:7-8, SEQ ID NOs:16-17, SEQ ID NOs:27-28, SEQ ID NOs:36-37, SEQ ID NOs:46-47, SEQ ID NOs:60-61, SEQ ID NOs:72-73, SEQ ID NOs:83-84, SEQ ID NOs:94-95, SEQ ID NOs:104-105, SEQ ID NOs:114-115, SEQ ID NOs:122-123, SEQ ID NOs:131-132, or SEQ ID NOs:139-140.

In some embodiments of the ADCs, the anti-CD180 binding molecule comprises a heavy chain and a light chain, said heavy chain and light chain comprise the amino acid sequences of SEQ ID NOs: 11-12, SEQ ID NOs:20-21, SEQ ID NOs:31-32, SEQ ID NOs:40-41, SEQ ID NOs:50-51, SEQ ID NOs:64-65, SEQ ID NOs:76-77, SEQ ID NOs:87-88, SEQ ID NOs:98-99, SEQ ID NOs:108-109, SEQ ID NOs:118-119, SEQ ID NOs:126-127, SEQ ID NOs:135-136, or SEQ ID NOs:143-144.

In certain embodiments of the anti-CD180 binding molecule, the binding molecule comprises an IgG, a Fv, a scFv, a Fab, a F(ab′)2, a minibody, a diabody, a triabody, a nanobody, a bispecific antibody, a single domain antibody, or a chimeric antigen receptor. In some embodiments, the binding molecule is an IgG, a Fv, a scFv, a Fab, a F(ab′)₂, a minibody, a diabody, a triabody, a nanobody, a bispecific antibody, a single domain antibody, or a chimeric antigen receptor. In an embodiment, of the anti-CD180 binding molecule, the IgG is IgG1, IgG2, IgG3, or IgG4. In an embodiment, the bispecific anti-CD180 antibody is a bi-paratopic antibody, i.e., a bispecific antibody that targets two non-overlapping epitopes on the same target antigen. In some embodiments, the bispecific antibody binds to a CD180 and a CD123 (“a CD180×CD123 bi-specific antibody”). In various embodiments, the bispecific anti-CD180 antibody comprises the variable heavy chain (VH) and variable light chain (VL) regions and/or the CDRs disclosed herein and the VH, VL, and/or the CDRs combinations disclosed herein that have binding specificity to CD180, i.e., comprises a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each comprise the amino acid sequences disclosed herein.

Also provided herein is an isolated polynucleotide sequence encoding the anti-CD180 binding molecules described herein. In an embodiment, a vector comprises the isolated polynucleotide sequence. In some embodiments, a host cell comprising the described vectors.

Further provided herein is a composition comprising any one of the herein provided anti-CD180 binding molecules and a pharmaceutically acceptable carrier. In some embodiments, the composition comprises an ADC, wherein the ADC comprises a CD180-high expressing tumor-targeting monoclonal antibody or antigen-binding fragment thereof, wherein the CD180-high expressing tumor-targeting monoclonal antibody or antigen-binding fragment thereof is an anti-CD180 antibody or antigen-binding fragment thereof comprising a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each respectively comprise the amino acid sequences:
HCDR1, HCDR2 and HCDR3 of SEQ ID NOs: 1-3 and LCDR1, LCDR2 and LCDR3 of SEQ ID NOs: 4-6; HCDR1, HCDR2 and HCDR3 of SEQ ID NOs: 1-3 and LCDR1, LCDR2 and LCDR3 of SEQ ID NOs: 4, 5, and 15;
HCDR1, HCDR2 and HCDR3 of SEQ ID NOs: 1, 24, and 25 and LCDR1, LCDR2 and LCDR3 of SEQ ID NOs: 4, 5, and 26;
HCDR1, HCDR2 and HCDR3 of SEQ ID NOs: 1, 35, and 25 and LCDR1, LCDR2 and LCDR3 of SEQ ID NOs: 4, 5, and 26;
HCDR1, HCDR2 and HCDR3 of SEQ ID NOs: 1, 44, and 25 and LCDR1, LCDR2 and LCDR3 of SEQ ID NOs: 4, 5, and 45;
HCDR1, HCDR2 and HCDR3 of SEQ ID NOs: 54-56 and LCDR1, LCDR2 and LCDR3 of SEQ ID NOs: 57-59;
HCDR1, HCDR2 and HCDR3 of SEQ ID NOs: 68-70 and LCDR1, LCDR2 and LCDR3 of SEQ ID NOs: 57, 58, and 71;
HCDR1, HCDR2 and HCDR3 of SEQ ID NOs: 1, 24, and 25 and LCDR1, LCDR2 and LCDR3 of SEQ ID NOs: 80-82;
HCDR1, HCDR2 and HCDR3 of SEQ ID NOs: 1, 35, and 25 and LCDR1, LCDR2 and LCDR3 of SEQ ID NOs: 91-93;
HCDR1, HCDR2 and HCDR3 of SEQ ID NOs: 1, 102, and 25 and LCDR1, LCDR2 and LCDR3 of SEQ ID NOs: 4, 103, and 6;
HCDR1, HCDR2 and HCDR3 of SEQ ID NOs: 1, 24, and 25 and LCDR1, LCDR2 and LCDR3 of SEQ ID NOs: 112, 5, and 113;
HCDR1, HCDR2 and HCDR3 of SEQ ID NOs: 1, 35, and 25 and LCDR1, LCDR2 and LCDR3 of SEQ ID NOs: 4-6;
HCDR1, HCDR2 and HCDR3 of SEQ ID NOs: 1, 44, and 25 and LCDR1, LCDR2 and LCDR3 of SEQ ID NOs: 4, 5, and 130; or
HCDR1, HCDR2 and HCDR3 of SEQ ID NOs: 1, 24, and 25 and LCDR1, LCDR2 and LCDR3 of SEQ ID NOs: 4-6.

Also provided herein is a composition comprising any one of the herein-provided ADCs and a pharmaceutically acceptable carrier. The compositions comprise an ADC comprising a CD180-high expressing tumor-targeting monoclonal antibody or antigen-binding fragment thereof, wherein the CD180-high expressing tumor-targeting monoclonal antibody or antigen-binding fragment thereof is an anti-CD180 antibody or antigen-binding fragment thereof comprising a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each respectively comprise the amino acid sequences:
HCDR1, HCDR2 and HCDR3 of SEQ ID NOs: 1-3 and LCDR1, LCDR2 and LCDR3 of SEQ ID NOs: 4-6;
HCDR1, HCDR2 and HCDR3 of SEQ ID NOs: 1-3 and LCDR1, LCDR2 and LCDR3 of SEQ ID NOs: 4, 5, and 15;
HCDR1, HCDR2 and HCDR3 of SEQ ID NOs: 1, 24, and 25 and LCDR1, LCDR2 and LCDR3 of SEQ ID NOs: 4, 5, and 26;
HCDR1, HCDR2 and HCDR3 of SEQ ID NOs: 1, 35, and 25 and LCDR1, LCDR2 and LCDR3 of SEQ ID NOs: 4, 5, and 26;
HCDR1, HCDR2 and HCDR3 of SEQ ID NOs: 1, 44, and 25 and LCDR1, LCDR2 and LCDR3 of SEQ ID NOs: 4, 5, and 45;
HCDR1, HCDR2 and HCDR3 of SEQ ID NOs: 54-56 and LCDR1, LCDR2 and LCDR3 of SEQ ID NOs: 57-59;
HCDR1, HCDR2 and HCDR3 of SEQ ID NOs: 68-70 and LCDR1, LCDR2 and LCDR3 of SEQ ID NOs: 57, 58, and 71;
HCDR1, HCDR2 and HCDR3 of SEQ ID NOs: 1, 24, and 25 and LCDR1, LCDR2 and LCDR3 of SEQ ID NOs: 80-82;
HCDR1, HCDR2 and HCDR3 of SEQ ID NOs: 1, 35, and 25 and LCDR1, LCDR2 and LCDR3 of SEQ ID NOs: 91-93;
HCDR1, HCDR2 and HCDR3 of SEQ ID NOs: 1, 102, and 25 and LCDR1, LCDR2 and LCDR3 of SEQ ID NOs: 4, 103, and 6;
HCDR1, HCDR2 and HCDR3 of SEQ ID NOs: 1, 24, and 25 and LCDR1, LCDR2 and LCDR3 of SEQ ID NOs: 112, 5, and 113;
HCDR1, HCDR2 and HCDR3 of SEQ ID NOs: 1, 35, and 25 and LCDR1, LCDR2 and LCDR3 of SEQ ID NOs: 4-6;
HCDR1, HCDR2 and HCDR3 of SEQ ID NOs: 1, 44, and 25 and LCDR1, LCDR2 and LCDR3 of SEQ ID NOs: 4, 5, and 130; or
HCDR1, HCDR2 and HCDR3 of SEQ ID NOs: 1, 24, and 25 and LCDR1, LCDR2 and LCDR3 of SEQ ID NOs: 4-6.

Further provided herein is a composition comprising the polynucleotide sequence encoding the above-described anti-CD180 binding molecules. In an embodiment, the polynucleotide sequence comprises an expression vector for expressing the anti-CD180 binding molecule in a cell.

The present disclosure provides a method of modulating CD180-mediated immune responses in a subject, comprising the step of administering to the subject any of the herein-provided compositions. In an embodiment, the CD180-mediated immune responses are antigen presenting cell activities of B cells, antibody secretion, or proliferation of B lymphocytes, monocytes or dendritic cells. In some embodiments, modulation of CD180-mediated immune responses comprises enhancing said CD180-mediated immune responses or inhibiting said CD180-mediated immune responses.

The present disclosure provides a method of treating a disease in a subject, comprising the step of administering to the subject any of the herein-provided compositions. In an embodiment, the disease is a viral infection, a bacterial infection, cancer, an autoimmune disease or an immune disorder. In a particular embodiment, the disease is a CD180-mediated disease. In an embodiment, the cancer is acute myeloid leukemia (AML), mantle cell lymphoma (MCL), multiple myeloma. follicular lymphoma, B-acute lymphoblastic leukemia, or diffuse large B-cell lymphoma.

The present disclosure provides a method of treating a CD180 high expressing cancer in a subject, comprising the step of administering to the subject any one of the herein-provided compositions comprising one of the described ADCs and a pharmaceutically acceptable carrier. In an embodiment, of the provided method, the ADC comprises a CD180-high expressing tumor-targeting monoclonal antibody or antigen-binding fragment thereof is an anti-CD180 antibody or antigen-binding fragment thereof comprising a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each respectively comprise the amino acid sequences:
HCDR1, HCDR2 and HCDR3 of SEQ ID NOs: 1-3 and LCDR1, LCDR2 and LCDR3 of SEQ ID NOs: 4-6;
HCDR1, HCDR2 and HCDR3 of SEQ ID NOs: 1-3 and LCDR1, LCDR2 and LCDR3 of SEQ ID NOs: 4, 5, and 15; HCDR1, HCDR2 and HCDR3 of SEQ ID NOs: 1, 24, and 25 and LCDR1, LCDR2 and LCDR3 of SEQ ID NOs: 4, 5, and 26;
HCDR1, HCDR2 and HCDR3 of SEQ ID NOs: 1, 35, and 25 and LCDR1, LCDR2 and LCDR of SEQ ID NOs: 4, 5, and 26;
HCDR1, HCDR2 and HCDR3 of SEQ ID NOs: 1, 44, and 25 and LCDR1, LCDR2 and LCDR3 of SEQ ID NOs: 4, 5, and 45;
HCDR1, HCDR2 and HCDR3 of SEQ ID NOs: 54-56 and LCDR1, LCDR2 and LCDR3 of SEQ ID NOs: 57-59;
HCDR1, HCDR2 and HCDR3 of SEQ ID NOs: 68-70 and LCDR1, LCDR2 and LCDR3 of SEQ ID NOs: 57, 58, and 71;
HCDR1, HCDR2 and HCDR3 of SEQ ID NOs: 1, 24, and 25 and LCDR1, LCDR2 and LCDR3 of SEQ ID NOs: 80-82;
HCDR1, HCDR2 and HCDR3 of SEQ ID NOs: 1, 35, and 25 and LCDR1, LCDR2 and LCDR3 of SEQ ID NOs: 91-93;
HCDR1, HCDR2 and HCDR3 of SEQ ID NOs: 1, 102, and 25 and LCDR1, LCDR2 and LCDR3 of SEQ ID NOs: 4, 103, and 6;
HCDR1, HCDR2 and HCDR3 of SEQ ID NOs: 1, 24, and 25 and LCDR1, LCDR2 and LCDR3 of SEQ ID NOs: 112, 5, and 113;
HCDR1, HCDR2 and HCDR3 of SEQ ID NOs: 1, 35, and 25 and LCDR1, LCDR2 and LCDR3 of SEQ ID NOs: 4-6;
HCDR1, HCDR2 and HCDR3 of SEQ ID NOs: 1, 44, and 25 and LCDR1, LCDR2 and LCDR3 of SEQ ID NOs: 4, 5, and 130; or
HCDR1, HCDR2 and HCDR3 of SEQ ID NOs: 1, 24, and 25 and LCDR1, LCDR2 and LCDR3 of SEQ ID NOs: 4-6.

The present disclosure provides an method of treating a CD180 high expressing cancer in a subject, comprising the step of administering to the subject any one of the herein described compositions comprising any one of the ADCs comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the cytotoxic drug payload comprises a ADC drug-linker conjugate tesirine (SG3249), the tesirine comprising a cytotoxic drug payload, the cytotoxic drug payload comprising pyrrolobenzodiazepine dimer cytotoxic DNA-alkylating agent (SG3199), wherein the tesirine is conjugated to the CD180-high expressing tumor-targeting monoclonal antibody or antigen-binding fragment thereof via a cleavable linker moiety. wherein the CD180-high expressing tumor-targeting monoclonal antibody or antigen-binding fragment thereof is an anti-CD180 antibody or antigen-binding fragment thereof comprising a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each respectively comprise the amino acid sequences:
HCDR1, HCDR2 and HCDR3 of SEQ ID NOs: 1-3 and LCDR1, LCDR2 and LCDR3 of SEQ ID NOs: 4-6;
HCDR1, HCDR2 and HCDR3 of SEQ ID NOs: 1-3 and LCDR1, LCDR2 and LCDR3 of SEQ ID NOs: 4, 5, and 15;
HCDR1, HCDR2 and HCDR3 of SEQ ID NOs: 1, 24, and 25 and LCDR1, LCDR2 and LCDR3 of SEQ ID NOs: 4, 5, and 26;
HCDR1, HCDR2 and HCDR3 of SEQ ID NOs: 1, 35, and 25 and LCDR1, LCDR2 and LCDR3 of SEQ ID NOs: 4, 5, and 26;
HCDR1, HCDR2 and HCDR3 of SEQ ID NOs: 1, 44, and 25 and LCDR1, LCDR2 and LCDR3 of SEQ ID NOs: 4, 5, and 45;
HCDR1, HCDR2 and HCDR3 of SEQ ID NOs: 54-56 and LCDR1, LCDR2 and LCDR3 of SEQ ID NOs: 57-59;
HCDR1, HCDR2 and HCDR3 of SEQ ID NOs: 68-70 and LCDR1, LCDR2 and LCDR3 of SEQ ID NOs: 57, 58, and 71;
HCDR1, HCDR2 and HCDR3 of SEQ ID NOs: 1, 24, and 25 and LCDR1, LCDR2 and LCDR3 of SEQ ID NOs: 80-82;
HCDR1, HCDR2 and HCDR3 of SEQ ID NOs: 1, 35, and 25 and LCDR1, LCDR2 and LCDR3 of SEQ ID NOs: 91-93;
HCDR1, HCDR2 and HCDR3 of SEQ ID NOs: 1, 102, and 25 and LCDR1, LCDR2 and LCDR3 of SEQ ID NOs: 4, 103, and 6;
HCDR1, HCDR2 and HCDR3 of SEQ ID NOs: 1, 24, and 25 and LCDR1, LCDR2 and LCDR3 of SEQ ID NOs: 112, 5, and 113;
HCDR1, HCDR2 and HCDR3 of SEQ ID NOs: 1, 35, and 25 and LCDR1, LCDR2 and LCDR3 of SEQ ID NOs: 4-6;
HCDR1, HCDR2 and HCDR3 of SEQ ID NOs: 1, 44, and 25 and LCDR1, LCDR2 and LCDR3 of SEQ ID NOs: 4, 5, and 130; or
HCDR1, HCDR2 and HCDR3 of SEQ ID NOs: 1, 24, and 25 and LCDR1, LCDR2 and LCDR3 of SEQ ID NOs: 4-6. In a particular embodiment, the cancer is acute myeloid leukemia (AML), mantle cell lymphoma (MCL), multiple myeloma, follicular lymphoma, B-acute lymphoblastic leukemia, or diffuse large B-cell lymphoma. In an embodiment, the cancer is primary AML. In some embodiments, the cancer is primary MCL.

The present disclosure provides a method of treating a CD180 high expressing cancer in a subject, comprising the step of administering to the subject composition the ADC comprising an ADC drug-linker conjugate deruxtecan, the deruxtecan comprising a cleavable linker, a self-immolative amino methylene spacer, and a cytotoxic drug payload, the cytotoxic drug payload comprising a topoisomerase 1 inhibitor payload, wherein the topoisomerase 1 inhibitor payload is a derivative of exatecan (DX-8951), wherein the deruxetan is conjugated to the CD180-high expressing tumor-targeting monoclonal antibody or antigen-binding fragment thereof via the cleavable linker, wherein the cleavable linker is a glycine-glycine-phenylalanine-glycine tetrapeptide-based linker. In an embodiment of the provided therapeutic methods, the ADC comprises a CD180-high expressing tumor-targeting monoclonal antibody or antigen-binding fragment thereof, wherein the CD180-high expressing tumor-targeting monoclonal antibody or antigen-binding fragment thereof is an anti-CD180 antibody or antigen-binding fragment thereof comprising a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each respectively comprise the amino acid sequences:
HCDR1, HCDR2 and HCDR3 of SEQ ID NOs: 1-3 and LCDR1, LCDR2 and LCDR3 of SEQ ID NOs: 4-6;
HCDR1, HCDR2 and HCDR3 of SEQ ID NOs: 1-3 and LCDR1, LCDR2 and LCDR3 of SEQ ID NOs: 4, 5, and 15;
HCDR1, HCDR2 and HCDR3 of SEQ ID NOs: 1, 24, and 25 and LCDR1, LCDR2 and LCDR3 of SEQ ID NOs: 4, 5, and 26;
HCDR1, HCDR2 and HCDR3 of SEQ ID NOs: 1, 35, and 25 and LCDR1, LCDR2 and LCDR3 of SEQ ID NOs: 4, 5, and 26;
HCDR1, HCDR2 and HCDR3 of SEQ ID NOs: 1, 44, and 25 and LCDR1, LCDR2 and LCDR3 of SEQ ID NOs: 4, 5, and 45;
HCDR1, HCDR2 and HCDR3 of SEQ ID NOs: 54-56 and LCDR1, LCDR2 and LCDR3 of SEQ ID NOs: 57-59;
HCDR1, HCDR2 and HCDR3 of SEQ ID NOs: 68-70 and LCDR1, LCDR2 and LCDR3 of SEQ ID NOs: 57, 58, and 71;
HCDR1, HCDR2 and HCDR3 of SEQ ID NOs: 1, 24, and 25 and LCDR1, LCDR2 and LCDR3 of SEQ ID NOs: 80-82;
HCDR1, HCDR2 and HCDR3 of SEQ ID NOs: 1, 35, and 25 and LCDR1, LCDR2 and LCDR3 of SEQ ID NOs: 91-93;
HCDR1, HCDR2 and HCDR3 of SEQ ID NOs: 1, 102, and 25 and LCDR1, LCDR2 and LCDR3 of SEQ ID NOs: 4, 103, and 6;
HCDR1, HCDR2 and HCDR3 of SEQ ID NOs: 1, 24, and 25 and LCDR1, LCDR2 and LCDR3 of SEQ ID NOs: 112, 5, and 113;
HCDR1, HCDR2 and HCDR3 of SEQ ID NOs: 1, 35, and 25 and LCDR1, LCDR2 and LCDR3 of SEQ ID NOs: 4-6;
HCDR1, HCDR2 and HCDR3 of SEQ ID NOs: 1, 44, and 25 and LCDR1, LCDR2 and LCDR3 of SEQ ID NOs: 4, 5, and 130; or
HCDR1, HCDR2 and HCDR3 of SEQ ID NOs: 1, 24, and 25 and LCDR1, LCDR2 and LCDR3 of SEQ ID NOs: 4-6. In some embodiments of the provided therapeutic methods, the cancer is acute myeloid leukemia (AML), mantle cell lymphoma (MCL), multiple myeloma, follicular lymphoma, B-acute lymphoblastic leukemia, or diffuse large B-cell lymphoma. In a particular embodiment, the cancer is primary AML. In certain embodiments of the described methods, the cancer is primary MCL.

### Methods of Use

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, “modulating” refers to “stimulating” or “inhibiting” an activity of a molecular target or pathway. For example, a composition modulates the activity of a molecular target or pathway if it stimulates or inhibits the activity of the molecular target or pathway by at least 10%, by at least about 20%, by at least about 25%, by at least about 30%, by at least about 40%, by at least about 50%, by at least about 60%, by at least about 70%, by at least about 75%, by at least about 80%, by at least about 90%, by at least about 95%, by at least about 98%, or by about 99% or more relative to the activity of the molecular target or pathway under the same conditions but lacking only the presence of the composition. In another example, a composition modulates the activity of a molecular target or pathway if it stimulates or inhibits the activity of the molecular target or pathway by at least 2-fold, at least 5-fold, at least 10-fold, at least 20-fold, at least 50-fold, at least 100-fold relative to the activity of the molecular target or pathway under the same conditions but lacking only the presence of the composition. The activity of a molecular target or pathway may be measured by any reproducible means. The activity of a molecular target or pathway may be measured in vitro or in vivo. For example, the activity of a molecular target or pathway may be measured in vitro or in vivo by an appropriate assay known in the art measuring the activity. Control samples (untreated with the composition) can be assigned a relative activity value of 100%.

In one embodiment, the present disclosure provides a method of modulating CD180-mediated immune responses in a subject, comprising the step of administering to the subject a composition comprising any one of the anti-CD180 binding molecules disclosed herein. In one embodiment, the composition comprises antibody drug conjugates as disclosed herein. In one embodiment, the antibody drug conjugates comprise tesirine. In another embodiment, the present disclosure provides a method of modulating CD180-mediated immune responses in a subject, comprising the step of administering to the subject a composition comprising any one of the polynucleotide sequences disclosed herein.

In one embodiment, the CD180-mediated immune responses comprise antigen presenting cell activities of B lineage cells. In another embodiment, the CD180-mediated immune responses comprise antibody secretion. In another embodiment, the CD180-mediated immune responses comprise proliferation of B lymphocytes, monocytes and/or dendritic cells.

In one embodiment, the above method of modulation comprises stimulating antigen presenting cell activities of B lineage cells. In another embodiment, the above method of modulation comprises inhibiting antigen presenting cell activities of B lineage cells. In one embodiment, the above method of modulation comprises stimulating antibody secretion (e.g. by B cells). In another embodiment, the above method of modulation comprises inhibiting antibody secretion (e.g., by B cells). In one embodiment, the above method of modulation comprises stimulating proliferation of B lymphocytes, monocytes and/or dendritic cells. In another embodiment, the above method of modulation comprises inhibiting proliferation of B lymphocytes, monocytes and/or dendritic cells.

In another embodiment, the present disclosure provides a method of treating a disease in a subject, comprising the step of administering to the subject a composition comprising any one of the anti-CD180 binding molecules disclosed herein. In another embodiment, the present disclosure provides a method of treating a disease in a subject, comprising the step of administering to the subject a composition comprising any one of the polynucleotide sequences disclosed herein. In one embodiment, the disease comprises any cancer or tumor cells that express CD180. In another embodiment, the disease comprises any disease for which the pathogenesis or treatment involves the functions or activities of antigen presenting cells. In another embodiment, the disease comprises any disease for which the pathogenesis or treatment involves antibody secretion by B cells. In one embodiment, examples of the cancer or diseases include, but are not limited to, acute myeloid leukemia, mantle cell lymphoma, multiple myeloma. follicular lymphoma, B-acute lymphoblastic leukemia, and diffuse large B-cell lymphoma.

In another embodiment, the anti-CD180 binding molecules or anti-CD180 antibodies disclosed herein can be used as an immunotherapy therapeutic to enhance or inhibit the activities of B cells as antigen presenting cells. In another embodiment, the anti-CD180 binding molecules or anti-CD180 antibodies disclosed herein can be used as an immunotherapy therapeutic to enhance or inhibit antibody secretion by B cells. In one embodiment, the immunotherapy therapeutic is useful for cancer or diseases such as, but are not limited to, acute myeloid leukemia, mantle cell lymphoma, multiple myeloma. follicular lymphoma, B-acute lymphoblastic leukemia, and diffuse large B-cell lymphoma.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The terms “subject,” “individual,” and “patient” are used interchangeably herein, and refer to human or non-human animals to whom treatment with a composition or formulation in accordance with the present disclosure is provided. The terms “non-human animals” and “non-human mammals” are used interchangeably herein and include all vertebrates, e.g., mammals, such as non-human primates (e.g., higher primates), sheep, dog, rodent (e.g. mouse or rat), guinea pig, goat, pig, cat, rabbits, cows, horses, or non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In one embodiment, the human is an adult. In another embodiment, the human is a child. The human can be male, female, pregnant, middle-aged, adolescent, or elderly.

Pharmaceutical compositions suitable for use in the methods disclosed herein include compositions wherein the active ingredients are contained in an amount effective to achieve the intended purpose. In one embodiment, a therapeutically effective amount means an amount of one or more active ingredients (e.g., the anti-CD180 binding molecules or antibodies) effective to prevent, alleviate or ameliorate symptoms of disease or prolong the survival of the subject being treated. Determination of a therapeutically effective amount is well within the capability of those skilled in the art.

In one embodiment, the exact amount of the present polypeptides or compositions thereof required to elicit the desired effects will vary from subject to subject, depending on the species, age, gender, weight, and general condition of the subject, the particular polypeptides, the route of administration, and whether other drugs are included in the regimen. Thus, it is not possible to specify an exact amount for every composition. However, an appropriate amount can be determined by one of ordinary skill in the art using routine experimentation. Dosages can vary, and the polypeptides can be administered in one or more (e.g., two or more, three or more, four or more, or five or more) doses daily, for one or more days. Guidance in selecting appropriate doses for antibodies can be readily found in the literature.

In one embodiment, the disease can be viral infection, bacterial infection, cancer, autoimmune disease or immune disorder. In one embodiment, the disease can be an upper respiratory viral infection, an early stage lung infection, or a late stage lung infection. A number of diseases and cancers are known to be caused by viruses. Examples of disease-causing viruses include, but are not limited to, norovirus; rotavirus; hepatitis virus A, B, C, D, or E; rabies virus, West Nile virus, enterovirus, echovirus, coxsackievirus, herpes simplex virus (HSV), HSV-2, varicella-zoster virus, mosquito-borne viruses, arbovirus, St. Louis encephalitis virus, California encephalitis virus, lymphocytic choriomeningitis virus, human immunodeficiency virus (HIV), poliovirus, zika virus, rubella virus, cytomegalovirus, human papillomavirus (HPV), enterovirus D68, severe acute respiratory syndrome (SARS) coronavirus, Middle East respiratory syndrome coronavirus, SARS coronavirus 2, Epstein-Barr virus, influenza virus, respiratory syncytial virus, polyoma viruses (such as JC virus, BK virus), Ebola virus, Dengue virus, or any combination thereof.

In another embodiment, the disease is a cancer that can be, but is not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoietic cancer, Non-Hodgkin lymphoma, skin cancer, basal-cell carcinoma, melanoma, small cell lung cancer, non-small cell lung cancer, or any combination thereof.

In another embodiment, the disease is an autoimmune disease that can be, but is not limited to, achalasia, amyloidosis, ankylosing spondylitis, anti-gbm/anti-tbm nephritis, antiphospholipid syndrome, arthritis, autoimmune angioedema, autoimmune encephalomyelitis, autoimmune hepatitis, autoimmune myocarditis, autoimmune oophoritis, autoimmune orchitis, autoimmune pancreatitis, autoimmune retinopathy, autoimmune urticaria, Bechet's disease, celiac disease, chagas disease, chronic inflammatory demyelinating polyneuropathy, Cogan's syndrome, congenital heart block, Crohn's disease, dermatitis, dermatomyositis, discoid lupus, Dressler's syndrome, endometriosis, fibromyalgia, fibrosing alveolitis, granulomatosis with polyangiitis, Graves' disease, Guillain-Barre syndrome, herpes gestationis, immune thrombocytopenic purpura, interstitial cystitis, juvenile arthritis, juvenile diabetes (type 1 diabetes), juvenile myositis, Kawasaki disease, Lambert-Eaton syndrome, lichen planus, lupus, Lyme disease, multiple sclerosis, myasthenia gravis, myositis, neonatal lupus, neutropenia, palindromic rheumatism, peripheral neuropathy, polyarteritis nodosa, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, reactive arthritis, retroperitoneal fibrosis, rheumatic fever, rheumatoid arthritis, sarcoidosis, Schmidt syndrome, scleritis, scleroderma, Sjögren's syndrome, thrombocytopenic purpura, type 1 diabetes, ulcerative colitis, uveitis, vasculitis, and vitiligo.

In some embodiments, the disease is a transplantation-related disease such as graft-versus-host disease (GvHD). According to one embodiment, the GVHD is acute GVHD. According to another embodiment, the GVHD is chronic GVHD.

Various embodiments and aspects of the present invention as delineated hereinabove and as claimed in the claims section below find experimental support in the following examples.

### EXAMPLES

### Example 1

### Immune Fab-Phage Library Constructions and Identification of Lead Anti-CD180 Antibodies

Using a proprietary Alloy Fab-phage display vector, an immune Fab-phage display library was generated from RNA of B cells from Alloy ATX-Gk™ mice that were immunized with human CD180 extracellular domain (ECD) HIS-tagged protein (Sino Biological, 11370-H08H; Creative Biomart, CD180-3900H). A second immune Fab-phage display library was generated from RNA of B cells of Alloy ATX-Gk™ mice that were immunized with human CD180/human MD-1 DNA (pDUO-hMD1/RP105, InvivoGen, pduo-hmd1rp105). Both Fab-Phage libraries were confirmed by Sanger sequencing (Sanger et al., Proc. Natl. Acad. Sci. 1977; 74:5463) to consist of Fabs with full-length VH/VK pairs.

Fab-phage from each library were enriched over 2 rounds for Fab specific CD180(ECD) binders by panning against recombinant CD180(ECD)-HIS proteins. For protein panning, CD180(ECD) binding Fab-phage were enriched for using biotinylated human or rhesus CD180(ECD)-HIS proteins (Sino Biological, 11370-H08H, Creative Biomart, CD180-1598R) with streptavidin magnetic beads (Invitrogen, 60210). To ensure captured Fab-phage were specific for CD180, protein panning with biotinylated CD180(ECD) proteins was performed in the presence of at least a 5-fold molar excess of a soluble CD180-like protein competitor complex consisting of human TLR4(ECD)-HIS/MD-2_HIS (R&D Systems, 3146-TM). TLR4's ECD has 28.2% sequence identity with human CD180(ECD). For each panning round, Fab-phage bound to CD180(ECD)-biotin-streptavidin-beads were eluted using 100 mM Triethylamine (TEA; Sigma-Aldrich, T0886) followed by neutralization with 1 M Tris, pH 8.0 (Invitrogen, AM9855G).

Cell panning was also performed on Fab-phage from each library over 2-3 rounds using HEK293 cells overexpressing human CD180/human MD-1 (pDUO-hMD1/RP105, InvivoGen, pduo-hmd1rp105) (Nagai Y. et al., 2002 Blood, 99(5):1699). Before panning, Fab-phage libraries were depleted to remove human MD-1 cross-reactive Fabs using human MD-1 overexpressing HEK293 cells (pUN01-hMD1, Invivogen, puno1-hmd1). For each panning round, Fab-phage that did not bind to MD-1 cells were panned against human CD180/human MD-1 overexpressing HEK 293 cells, and cell-bound Fab-phage eluted using 100 mM TEA, and neutralized with 1 M Tris, pH 8.0.

Protein and cell ELISA on polyclonal Fab-Phage isolated from protein and cell panning confirmed that Fab-phage were preferentially enriched against the reagent used for panning, i.e. Fab-phage isolated by human CD180(ECD)-HIS protein panning bound stronger to the recombinant protein than to human CD180/MD-1 overexpressing HEK293 cells. In addition, ELISA, using plate-immobilized recombinant proteins, confirmed that panned Fab-phage were enriched up to 40-fold against human CD180(ECD)-HIS and lacked reactivity with the human TLR4/MD-2 protein complex or with human MD-1-Fc (BON-OPUS, CJ56).

Having confirmed specific enrichment of polyclonal Fab-phage against the CD180 target, 1,920 monoclonal Fabs were expressed by infecting and growing the panned phage from the two libraries in SS320 E. coli, a bacterial strain that recognize an amber stop codon (Sidhu et al Methods in Enzymology, 2000; 328:333) which in the phage vector is located between Fab and a phage coat protein g3p. Protein and cell ELISA results for monoclonal Fab supernatants, generated using Triton™ X-100 (Sigma-Aldrich, T9284-1L) in the induction media, confirmed that 75% (1438) of Fabs were positive for the CD180 target. Sanger sequencing of the CD180 binding monoclonal Fabs confirmed high antibody diversity, which consisted of 134 HCDR3 clonotypes (each clonotype consisted of Fabs with at least 80% HCDR3 sequence identity), 24 VH and 16 VK genes. Two hundred nineteen (219) Fabs, which were representative of HCDR3 clonotypes and VH/VK gene diversity, were purified using HisPur™ Ni-NTA Magnetic Beads (Thermo Scientific, 88832) and tested by ELISA and flow cytometry for specificity for the CD180 target. Based on the latter results, 48 lead Fabs were chosen to be reformatted as IgG1s for further characterization of their anti-CD180 activity.

### Generation of a Panel of Antibodies to CD180

To generate antibody against CD180, 2 cohorts comprising each 5 ATX-GK mice were immunized. ATX-GK can produce human antibodies. Cohort 1 was weekly immunized subcutaneously with CD180 protein carrying an His-tag (Sino Biological, 11370-H08H; Creative Biomart, CD180-3900H) while cohort 2 was weekly immunized intradermally with a plasmid DNA containing human CD180/human MD-1 (pDUO-hMD1/RP105, InvivoGen, pduo-hmd1rp105). After a total of 5 injections for cohort 1 and 8 injections for cohort 2, mice were sacrificed and their splenocytes were used for hybridoma generation and immune-phage libraries.

For titer measurement, mouse sera were prepared and used in an ELISA against CD180 protein (titer assay #1) or in a cell-based binding assay by flow cytometry (titer assay #2). Briefly, for titer assay #1, CD180 protein was coated on an ELISA plate. Plates were blocked with 3% BSA buffer for 1 hr and after PBS wash, sera dilutions were transferred to the ELISA plate and incubated for 1 hr. After PBS wash, a secondary antibody specific for mouse IgG was added to the ELISA plate for 1 hr. After PBS wash, TMB solution was added, and reaction was then stopped using sulfuric acid.

For titer assay #2, sera dilutions were incubated with a cell line expressing CD180/MD1 complex for 1 hr. After PBS wash, a secondary antibody specific for mouse IgG was added to the plate and mean fluorescence intensity was measured using a flow cytometry.

For hybridoma generation, splenocytes were fused to a myeloma cell line using standard protocols to create hybridomas (Kohler & Milstein, Nature, 1975, 256:495). Individual hybridoma supernatants were screened by ELISA for binding to human CD180. Positive clones were then tested by flow cytometry for binding to cells expressing CD180 to select hybridomas with reactivity to native CD180.

Table 1 shows heavy chain CDR sequences (HCDR1, HCDR2, HCDR3) for various antibodies presented herein. Table 2 shows light chain CDR sequences (LCDR1, LCDR2, LCDR3) for various antibodies presented herein.

Table 3 shows amino acid sequences of heavy chain variable region (VH), light chain variable region (VL), heavy chain (HC), and light chain (LC) for various antibodies presented herein. Table 4 shows nucleotide sequences encoding the VH, VL, HC, and LC of various antibodies presented herein.

TABLE 1
Embodiments of Heavy Chain CDR Sequences

| | | SEQ | | SEQ | | SEQ |
|---|---|---|---|---|---|---|
| Ab ID | HCDR1 | ID NO | HCDR2 | ID NO | HCDR3 | ID NO |
| Ab-001 | DYAMH | 1 | YISWNNNNIGYADSV | 2 | DIAYNTGYFDY | 3 |
| | | | KG | | | |
| Ab-002 | DYAMH | 1 | YISWNNNNIGYADSV | 2 | DIAYNTGYFDY | 3 |
| | | | KG | | | |
| Ab-003 | DYAMH | 1 | GISWNGGTMGYADS | 24 | DIAYSRGYFQH | 25 |
| | | | VKG | | | |
| Ab-004 | DYAMH | 1 | GISWNGVTIDYADSV | 35 | DIAYSRGYFQH | 25 |
| | | | KG | | | |
| Ab-005 | DYAMH | 1 | GISWNGGTIGYADSV | 44 | DIAYSRGYFQH | 25 |
| | | | KG | | | |
| Ab-006 | DYDMT | 54 | GINWNGGNTDYADS | 55 | KGRDIVVVKGF | 56 |
| | | | VKG | | YYHYYYMDV | |
| Ab-007 | DYAMS | 68 | DINWNGGTTSYADS | 69 | KGRDTVVVKGY | 70 |
| | | | VKG | | YYFSYYMDV | |
| Ab-008 | DYAMH | 1 | GISWNGGTMGYADS | 24 | DIAYSRGYFQH | 25 |
| | | | VKG | | | |
| Ab-009 | DYAMH | 1 | GISWNGVTIDYADSV | 35 | DIAYSRGYFQH | 25 |
| | | | KG | | | |
| Ab-010 | DYAMH | 1 | GISWNGGTFDYADSV | 102 | DIAYSRGYFQH | 25 |
| | | | KG | | | |
| Ab-012 | DYAMH | 1 | GISWNGGTMGYADS | 24 | DIAYSRGYFQH | 25 |
| | | | VKG | | | |
| Ab-014 | DYAMH | 1 | GISWNGVTIDYADSV | 35 | DIAYSRGYFQH | 25 |
| | | | KG | | | |
| Ab-016 | DYAMH | 1 | GISWNGGTIGYADSV | 44 | DIAYSRGYFQH | 25 |
| | | | KG | | | |
| Ab-017 | DYAMH | 1 | GISWNGGTMGYADS | 24 | DIAYSRGYFQH | 25 |
| | | | VKG | | | |

TABLE 2
Embodiments of Light Chain CDR Sequences

| | | SEQ | | SEQ | | SEQ |
|---|---|---|---|---|---|---|
| Ab ID | LCDR1 | ID NO | LCDR2 | ID NO | LCDR3 | ID NO |
| Ab-001 | RASQSVSSSYLA | 4 | GASSRAT | 5 | QQYGSSPLT | 6 |
| Ab-002 | RASQSVSSSYLA | 4 | GASSRAT | 5 | QQYGSSPFT | 15 |
| Ab-003 | RASQSVSSSYLA | 4 | GASSRAT | 5 | QQYGSSPRT | 26 |
| Ab-004 | RASQSVSSSYLA | 4 | GASSRAT | 5 | QQYGSSPRT | 26 |
| Ab-005 | RASQSVSSSYLA | 4 | GASSRAT | 5 | QQYGSSPYT | 45 |
| Ab-006 | RSSQSLLHSNGYNY | 57 | LGSNRAS | 58 | MQALQTPLT | 59 |
| | LD | | | | | |
| Ab-007 | RSSQSLLHSNGYNY | 57 | LGSNRAS | 58 | MQALQTPWT | 71 |
| | LD | | | | | |
| Ab-008 | RASQGIRNDLG | 80 | AASTLQS | 81 | QKYNSAPFT | 82 |
| Ab-009 | KSSQNVLYSSNNEN | 91 | WASTRES | 92 | QQYYSTPYT | 93 |
| | HLA | | | | | |
| Ab-010 | RASQSVSSSYLA | 4 | GASTRAT | 103 | QQYGSSPLT | 6 |
| Ab-012 | RASQSVSSNLA | 112 | GASSRAT | 5 | QQYGSSQIT | 113 |
| Ab-014 | RASQSVSSSYLA | 4 | GASSRAT | 5 | QQYGSSPLT | 6 |
| Ab-016 | RASQSVSSSYLA | 4 | GASSRAT | 5 | QQYGSSPHT | 130 |
| Ab-017 | RASQSVSSSYLA | 4 | GASSRAT | 5 | QQYGSSPLT | 6 |

TABLE 3
Amino acid Sequences of VH, VL, HC, LC

| | | | SEQ |
|---|---|---|---|
| | | | ID |
| Ab ID | Name | Sequence | NO |
| Ab- | VH | EVQLVESGGGLVQPGGSLRLSCAASGFTSLDYAMHWVRQAPGKGL | 7 |
| 001 | | EWVSYISWNNNNIGYADSVKGRFTISRDNAKNSLYLHMNSLRPEDT | |
| | | AFYYCAKDIAYNTGYFDYWGQGTLVTVSS | |
| Ab- | VL | EIVLTQSPGTLSLSPGERATLSCRASQSVSSSYLAWYQQKPGQAPRLL | 8 |
| 001 | | IYGASSRATGIPDRFSGSGSGTDFTLTISRLEPEDFAVYYCQQYGSSPL | |
| | | TFGGGTKVEIK | |
| Ab- | HC | EVQLVESGGGLVQPGGSLRLSCAASGFTSLDYAMHWVRQAPGKGL | 11 |
| 001 | | EWVSYISWNNNNIGYADSVKGRFTISRDNAKNSLYLHMNSLRPEDT | |
| | | AFYYCAKDIAYNTGYFDYWGQGTLVTVSSASTKGPSVFPLAPSSKST | |
| | | SGGTAALGCLVKDYFPEPVTVSWNSGALTSGVHTFPAVLQSSGLYSL | |
| | | SSVVTVPSSSLGTQTYICNVNHKPSNTKVDKRVEPKSCDKTHTCPPC | |
| | | PAPELLGGPSVFLFPPKPKDTLMISRTPEVTCVVVDVSHEDPEVKEN | |
| | | WYVDGVEVHNAKTKPREEQYNSTYRVVSVLTVLHQDWLNGKEYK | |
| | | CKVSNKALPAPIEKTISKAKGQPREPQVYTLPPSREEMTKNQVSLTCL | |
| | | VKGFYPSDIAVEWESNGQPENNYKTTPPVLDSDGSFFLYSKLTVDKS | |
| | | RWQQGNVFSCSVMHEALHNHYTQKSLSLSPG | |
| Ab- | LC | EIVLTQSPGTLSLSPGERATLSCRASQSVSSSYLAWYQQKPGQAPRLL | 12 |
| 001 | | IYGASSRATGIPDRESGSGSGTDFTLTISRLEPEDFAVYYCQQYGSSPL | |
| | | TFGGGTKVEIKRTVAAPSVFIFPPSDEQLKSGTASVVCLLNNFYPREA | |
| | | KVQWKVDNALQSGNSQESVTEQDSKDSTYSLSSTLTLSKADYEKHK | |
| | | VYACEVTHQGLSSPVTKSFNRGEC | |
| Ab- | VH | EVQLVESGGGLVQPGGSLRLSCAASGFTSVDYAMHWVRQAPGKGL | 16 |
| 002 | | EWVSYISWNNNNIGYADSVKGRFTISRDNAKNSLYLHMSSLRAEDT | |
| | | ALYYCAKDIAYNTGYFDYWGQGTLVTVSS | |
| Ab- | VL | EIVLTQSPGTLSLSPGERATLSCRASQSVSSSYLAWYQQKPGQAPRLL | 17 |
| 002 | | IYGASSRATGIPDRFSGSGSGTDFTLTISRLEPEDFAVYYCQQYGSSPF | |
| | | TFGPGTKVEIK | |
| Ab- | HC | EVQLVESGGGLVQPGGSLRLSCAASGFTSVDYAMHWVRQAPGKGL | 20 |
| 002 | | EWVSYISWNNNNIGYADSVKGRFTISRDNAKNSLYLHMSSLRAEDT | |
| | | ALYYCAKDIAYNTGYFDYWGQGTLVTVSSASTKGPSVFPLAPSSKST | |
| | | SGGTAALGCLVKDYFPEPVTVSWNSGALTSGVHTFPAVLQSSGLYSL | |
| | | SSVVTVPSSSLGTQTYICNVNHKPSNTKVDKRVEPKSCDKTHTCPPC | |
| | | PAPELLGGPSVFLFPPKPKDTLMISRTPEVTCVVVDVSHEDPEVKEN | |
| | | WYVDGVEVHNAKTKPREEQYNSTYRVVSVLTVLHQDWLNGKEYK | |
| | | CKVSNKALPAPIEKTISKAKGQPREPQVYTLPPSREEMTKNQVSLTCL | |
| | | VKGFYPSDIAVEWESNGQPENNYKTTPPVLDSDGSFFLYSKLTVDKS | |
| | | RWQQGNVFSCSVMHEALHNHYTQKSLSLSPG | |
| Ab- | LC | EIVLTQSPGTLSLSPGERATLSCRASQSVSSSYLAWYQQKPGQAPRLL | 21 |
| 002 | | IYGASSRATGIPDRFSGSGSGTDFTLTISRLEPEDFAVYYCQQYGSSPF | |
| | | TFGPGTKVEIKRTVAAPSVFIFPPSDEQLKSGTASVVCLLNNFYPREA | |
| | | KVQWKVDNALQSGNSQESVTEQDSKDSTYSLSSTLTLSKADYEKHK | |
| | | VYACEVTHQGLSSPVTKSFNRGEC | |
| Ab- | VH | EVQLVESGGGLVQPGRSLRLSCTTSGFTFADYAMHWVRQTPGQGLE | 27 |
| 003 | | WVSGISWNGGTMGYADSVKGRFTISRDIAKNSLYLQMNSLRAEDTA | |
| | | LYYCAKDIAYSRGYFQHWGQGTLVTVSS | |
| Ab- | VL | EIVLTQSPGTLSLSPGERATLSCRASQSVSSSYLAWYQQKPGQAPRLL | 28 |
| 003 | | IYGASSRATGIPDRFSGSGSGTDFTLTISRLEPEDFAVYYCQQYGSSPR | |
| | | TFGQGTKVEIK | |
| Ab- | HC | EVQLVESGGGLVQPGRSLRLSCTTSGFTFADYAMHWVRQTPGQGLE | 31 |
| 003 | | WVSGISWNGGTMGYADSVKGRFTISRDIAKNSLYLQMNSLRAEDTA | |
| | | LYYCAKDIAYSRGYFQHWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKRVEPKSCDKTHTCPPCP | |
| | | APELLGGPSVFLFPPKPKDTLMISRTPEVTCVVVDVSHEDPEVKFNW | |
| | | YVDGVEVHNAKTKPREEQYNSTYRVVSVLTVLHQDWLNGKEYKC | |
| | | KVSNKALPAPIEKTISKAKGQPREPQVYTLPPSREEMTKNQVSLTCLV | |
| | | KGFYPSDIAVEWESNGQPENNYKTTPPVLDSDGSFFLYSKLTVDKSR | |
| | | WQQGNVFSCSVMHEALHNHYTQKSLSLSPG | |
| Ab- | LC | EIVLTQSPGTLSLSPGERATLSCRASQSVSSSYLAWYQQKPGQAPRLL | 32 |
| 003 | | IYGASSRATGIPDRFSGSGSGTDFTLTISRLEPEDFAVYYCQQYGSSPR | |
| | | TFGQGTKVEIKRTVAAPSVFIFPPSDEQLKSGTASVVCLLNNFYPREA | |
| | | KVQWKVDNALQSGNSQESVTEQDSKDSTYSLSSTLTLSKADYEKHK | |
| | | VYACEVTHQGLSSPVTKSFNRGEC | |
| Ab- | VH | EVQLVESGGGLVQPGGSLRLSCTTSGFTFADYAMHWVRQTPGKGLE | 36 |
| 004 | | WVSGISWNGVTIDYADSVKGRFTISRDIAKNSLYLQMNSLRAEDTAL | |
| | | YYCAKDIAYSRGYFQHWGQGTLVTVSS | |
| Ab- | VL | EIVLTQSPATLSVSPGERATLSCRASQSVSSSYLAWYQQKPGQAPRLL | 37 |
| 004 | | IYGASSRATGIPDRESGSGSGTDFTLTISRLEPEDFAVYYCQQYGSSPR | |
| | | TFGQGTKVEIK | |
| Ab | HC | EVQLVESGGGLVQPGGSLRLSCTTSGFTFADYAMHWVRQTPGKGLE | 40 |
| 004 | | WVSGISWNGVTIDYADSVKGRFTISRDIAKNSLYLQMNSLRAEDTAL | |
| | | YYCAKDIAYSRGYFQHWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKRVEPKSCDKTHTCPPCPA | |
| | | PELLGGPSVFLFPPKPKDTLMISRTPEVTCVVVDVSHEDPEVKFNWY | |
| | | VDGVEVHNAKTKPREEQYNSTYRVVSVLTVLHQDWLNGKEYKCK | |
| | | VSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRW | |
| | | QQGNVFSCSVMHEALHNHYTQKSLSLSPG | |
| Ab- | LC | EIVLTQSPATLSVSPGERATLSCRASQSVSSSYLAWYQQKPGQAPRLL | 41 |
| 004 | | IYGASSRATGIPDRFSGSGSGTDFTLTISRLEPEDFAVYYCQQYGSSPR | |
| | | TFGQGTKVEIKRTVAAPSVFIFPPSDEQLKSGTASVVCLLNNFYPREA | |
| | | KVQWKVDNALQSGNSQESVTEQDSKDSTYSLSSTLTLSKADYEKHK | |
| | | VYACEVTHQGLSSPVTKSFNRGEC | |
| Ab- | VH | EVQLVESGGGLVQPGGSLRLSCTTSGFTFADYAMHWVRQTPGKGLE | 46 |
| 005 | | WVSGISWNGGTIGYADSVKGRFTISRDIAKNSLYLQMNSLRAEDTAL | |
| | | YYCAKDIAYSRGYFQHWGQGTLVTVSS | |
| Ab- | VL | EIVLTQSPGTLSLSPGERATLSCRASQSVSSSYLAWYQQKPGQAPRLL | 47 |
| 005 | | IYGASSRATGIPDRFSGSGSGTDFTLTISRLEPEDFAVYYCQQYGSSPY | |
| | | TFGQGTKVDIK | |
| Ab- | HC | EVQLVESGGGLVQPGGSLRLSCTTSGFTFADYAMHWVRQTPGKGLE | 50 |
| 005 | | WVSGISWNGGTIGYADSVKGRFTISRDIAKNSLYLQMNSLRAEDTAL | |
| | | YYCAKDIAYSRGYFQHWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKRVEPKSCDKTHTCPPCPA | |
| | | PELLGGPSVFLFPPKPKDTLMISRTPEVTCVVVDVSHEDPEVKFNWY | |
| | | VDGVEVHNAKTKPREEQYNSTYRVVSVLTVLHQDWLNGKEYKCK | |
| | | VSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRW | |
| | | QQGNVFSCSVMHEALHNHYTQKSLSLSPG | |
| Ab- | LC | EIVLTQSPGTLSLSPGERATLSCRASQSVSSSYLAWYQQKPGQAPRLL | 51 |
| 005 | | IYGASSRATGIPDRFSGSGSGTDFTLTISRLEPEDFAVYYCQQYGSSPY | |
| | | TFGQGTKVDIKRTVAAPSVFIFPPSDEQLKSGTASVVCLLNNFYPREA | |
| | | KVQWKVDNALQSGNSQESVTEQDSKDSTYSLSSTLTLSKADYEKHK | |
| | | VYACEVTHQGLSSPVTKSFNRGEC | |
| Ab- | VH | EVQLVESGGGLVKPGGSLRLSCTASGFTFDDYDMTWVRQAPGKGLE | 60 |
| 006 | | WVSGINWNGGNTDYADSVKGRFTISRDNTKNSLYLQMNSLRAEDT | |
| | | ALYYCARKGRDIVVVKGFYYHYYYMDVWGKGITVTVSS | |
| Ab- | VL | DIVMTQSPLSLPVTPGEPASISCRSSQSLLHSNGYNYLDWYLQKPGQS | 61 |
| 006 | | PQLLIYLGSNRASGVPDRFSGSGSGTDFTLKISRVEAEDVGVYYCMQ | |
| | | ALQTPLTFGGGTKVDIK | |
| Ab- | HC | EVQLVESGGGLVKPGGSLRLSCTASGFTFDDYDMTWVRQAPGKGLE | 64 |
| 006 | | WVSGINWNGGNTDYADSVKGRFTISRDNTKNSLYLQMNSLRAEDT | |
| | | ALYYCARKGRDIVVVKGFYYHYYYMDVWGKGITVTVSSASTKGPS | |
| | | VFPLAPSSKSTSGGTAALGCLVKDYFPEPVTVSWNSGALTSGVHTFP | |
| | | AVLQSSGLYSLSSVVTVPSSSLGTQTYICNVNHKPSNTKVDKRVEPK | |
| | | SCDKTHTCPPCPAPELLGGPSVFLFPPKPKDTLMISRTPEVTCVVVDV | |
| | | SHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEM | |
| | | TKNQVSLTCLVKGFYPSDIAVEWESNGQPENNYKTTPPVLDSDGSFF | |
| | | LYSKLTVDKSRWQQGNVFSCSVMHEALHNHYTQKSLSLSPG | |
| Ab- | LC | DIVMTQSPLSLPVTPGEPASISCRSSQSLLHSNGYNYLDWYLQKPGQS | 65 |
| 006 | | PQLLIYLGSNRASGVPDRFSGSGSGTDFTLKISRVEAEDVGVYYCMQ | |
| | | ALQTPLTFGGGTKVDIKRTVAAPSVFIFPPSDEQLKSGTASVVCLLNN | |
| | | FYPREAKVQWKVDNALQSGNSQESVTEQDSKDSTYSLSSTLTLSKA | |
| | | DYEKHKVYACEVTHQGLSSPVTKSFNRGEC | |
| Ab- | VH | EVQLVESGGGVVRPGGSLRLSCAASGFTFDDYAMSWVRQAPGKGL | 72 |
| 007 | | EWVSDINWNGGTTSYADSVKGRFTISRDNAKNSLFLQMNSLRAEDT | |
| | | ALYYCARKGRDTVVVKGYYYFSYYMDVWGKGSTVTVSS | |
| Ab- | VL | DIVMTQSPLSLPVTPGEPASISCRSSQSLLHSNGYNYLDWYLQKPGQS | 73 |
| 007 | | PQLLIYLGSNRASGVPDRFSGSGSGTDFTLKISRVEAEDVGVYYCMQ | |
| | | ALQTPWTFGQGTKLEIK | |
| Ab- | HC | EVQLVESGGGVVRPGGSLRLSCAASGFTFDDYAMSWVRQAPGKGL | 76 |
| 007 | | EWVSDINWNGGTTSYADSVKGRFTISRDNAKNSLFLQMNSLRAEDT | |
| | | ALYYCARKGRDTVVVKGYYYFSYYMDVWGKGSTVTVSSASTKGPS | |
| | | VFPLAPSSKSTSGGTAALGCLVKDYFPEPVTVSWNSGALTSGVHTFP | |
| | | AVLQSSGLYSLSSVVTVPSSSLGTQTYICNVNHKPSNTKVDKRVEPK | |
| | | SCDKTHTCPPCPAPELLGGPSVFLFPPKPKDTLMISRTPEVTCVVVDV | |
| | | SHEDPEVKFNWYVDGVEVHNAKTKPREEQYNSTYRVVSVLTVLHQ | |
| | | DWLNGKEYKCKVSNKALPAPIEKTISKAKGQPREPQVYTLPPSREEM | |
| | | TKNQVSLTCLVKGFYPSDIAVEWESNGQPENNYKTTPPVLDSDGSFF | |
| | | LYSKLTVDKSRWQQGNVFSCSVMHEALHNHYTQKSLSLSPG | |
| Ab- | LC | DIVMTQSPLSLPVTPGEPASISCRSSQSLLHSNGYNYLDWYLQKPGQS | 77 |
| 007 | | PQLLIYLGSNRASGVPDRFSGSGSGTDFTLKISRVEAEDVGVYYCMQ | |
| | | ALQTPWTFGQGTKLEIKRTVAAPSVFIFPPSDEQLKSGTASVVCLLNN | |
| | | FYPREAKVQWKVDNALQSGNSQESVTEQDSKDSTYSLSSTLTLSKA | |
| | | DYEKHKVYACEVTHQGLSSPVTKSFNRGEC | |
| Ab- | VH | EVQLVESGGGLVQPGRSLRLSCTTSGFTFADYAMHWVRQTPGQGLE | 83 |
| 008 | | WVSGISWNGGTMGYADSVKGRFTISRDIAKNSLYLQMNSLRAEDTA | |
| | | LYYCAKDIAYSRGYFQHWGQGTLVTVSS | |
| Ab- | VL | DIQMTQSPSSLSASVGDRVTITCRASQGIRNDLGWYQQKPGKAPKLL | 84 |
| 008 | | IYAASTLQSGVPSRFSGSGSGTDFTLTISSLQPEDVATYYCQKYNSAP | |
| | | FTFGPGTKVDIK | |
| Ab- | HC | EVQLVESGGGLVQPGRSLRLSCTTSGFTFADYAMHWVRQTPGQGLE | 87 |
| 008 | | WVSGISWNGGTMGYADSVKGRFTISRDIAKNSLYLQMNSLRAEDTA | |
| | | LYYCAKDIAYSRGYFQHWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKRVEPKSCDKTHTCPPCP | |
| | | APELLGGPSVFLFPPKPKDTLMISRTPEVTCVVVDVSHEDPEVKFNW | |
| | | YVDGVEVHNAKTKPREEQYNSTYRVVSVLTVLHQDWLNGKEYKC | |
| | | KVSNKALPAPIEKTISKAKGQPREPQVYTLPPSREEMTKNQVSLTCLV | |
| | | KGFYPSDIAVEWESNGQPENNYKTTPPVLDSDGSFFLYSKLTVDKSR | |
| | | WQQGNVFSCSVMHEALHNHYTQKSLSLSPG | |
| Ab- | LC | DIQMTQSPSSLSASVGDRVTITCRASQGIRNDLGWYQQKPGKAPKLL | 88 |
| 008 | | IYAASTLQSGVPSRFSGSGSGTDFTLTISSLQPEDVATYYCQKYNSAP | |
| | | FTFGPGTKVDIKRTVAAPSVFIFPPSDEQLKSGTASVVCLLNNFYPRE | |
| | | AKVQWKVDNALQSGNSQESVTEQDSKDSTYSLSSTLTLSKADYEKH | |
| | | KVYACEVTHQGLSSPVTKSFNRGEC | |
| Ab- | VH | EVQLVESGGGLVQPGRSLRLSCTTSGFTFADYAMHWVRQTPGKGLE | 94 |
| 009 | | WVSGISWNGVTIDYADSVKGRFTISRDIAKNSLYLQMNSLRAEDTAL | |
| | | YYCAKDIAYSRGYFQHWGQGTLVTVSS | |
| Ab- | VL | DIVMTQSPDSLAVSLGERATVNCKSSQNVLYSSNNENHLAWYQQKP | 95 |
| 009 | | GQPPKLLIYWASTRESGVPDRFSGSGSGTDFTLTISSLQAEDVAIYYC | |
| | | QQYYSTPYTFGQGTKVDIK | |
| Ab- | HC | EVQLVESGGGLVQPGRSLRLSCTTSGFTFADYAMHWVRQTPGKGLE | 98 |
| 009 | | WVSGISWNGVTIDYADSVKGRFTISRDIAKNSLYLQMNSLRAEDTAL | |
| | | YYCAKDIAYSRGYFQHWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKRVEPKSCDKTHTCPPCPA | |
| | | PELLGGPSVFLFPPKPKDTLMISRTPEVTCVVVDVSHEDPEVKFNWY | |
| | | VDGVEVHNAKTKPREEQYNSTYRVVSVLTVLHQDWLNGKEYKCK | |
| | | VSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRW | |
| | | QQGNVFSCSVMHEALHNHYTQKSLSLSPG | |
| Ab- | LC | DIVMTQSPDSLAVSLGERATVNCKSSQNVLYSSNNENHLAWYQQKP | 99 |
| 009 | | GQPPKLLIYWASTRESGVPDRFSGSGSGTDFTLTISSLQAEDVAIYYC | |
| | | QQYYSTPYTFGQGTKVDIKRTVAAPSVFIFPPSDEQLKSGTASVVCLL | |
| | | NNFYPREAKVQWKVDNALQSGNSQESVTEQDSKDSTYSLSSTLTLS | |
| | | KADYEKHKVYACEVTHQGLSSPVTKSFNRGEC | |
| Ab- | VH | EVQLVESGGGLVQPGGSLRLSCTSSGFTFADYAMHWVRQTPGKGLE | 104 |
| 010 | | WVSGISWNGGTFDYADSVKGRFTISRDIAKNSLYLQMNSLRVEDTA | |
| | | LYYCAKDIAYSRGYFQHWGQGTLVTVSS | |
| Ab- | VL | EIVLTQSPGTLSLSPGERATLSCRASQSVSSSYLAWYQQKPGQAPRLL | 105 |
| 010 | | IYGASTRATGIPDRFSGSGSGTDFTLTISRLEPEDFAVYYCQQYGSSPL | |
| | | TFGGGTKVEIK | |
| Ab- | HC | EVQLVESGGGLVQPGGSLRLSCTSSGFTFADYAMHWVRQTPGKGLE | 108 |
| 010 | | WVSGISWNGGTFDYADSVKGRFTISRDIAKNSLYLQMNSLRVEDTA | |
| | | LYYCAKDIAYSRGYFQHWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKRVEPKSCDKTHTCPPCP | |
| | | APELLGGPSVFLFPPKPKDTLMISRTPEVTCVVVDVSHEDPEVKFNW | |
| | | YVDGVEVHNAKTKPREEQYNSTYRVVSVLTVLHQDWLNGKEYKC | |
| | | KVSNKALPAPIEKTISKAKGQPREPQVYTLPPSREEMTKNQVSLTCLV | |
| | | KGFYPSDIAVEWESNGQPENNYKTTPPVLDSDGSFFLYSKLTVDKSR | |
| | | WQQGNVFSCSVMHEALHNHYTQKSLSLSPG | |
| Ab- | LC | EIVLTQSPGTLSLSPGERATLSCRASQSVSSSYLAWYQQKPGQAPRLL | 109 |
| 010 | | IYGASTRATGIPDRFSGSGSGTDFTLTISRLEPEDFAVYYCQQYGSSPL | |
| | | TFGGGTKVEIKRTVAAPSVFIFPPSDEQLKSGTASVVCLLNNFYPREA | |
| | | KVQWKVDNALQSGNSQESVTEQDSKDSTYSLSSTLTLSKADYEKHK | |
| | | VYACEVTHQGLSSPVTKSFNRGEC | |
| Ab- | VH | EVQLVESGGGLVQPGRSLRLSCTTSGFTFADYAMHWVRQTPGQGLE | 114 |
| 012 | | WVSGISWNGGTMGYADSVKGRFTISRDIAKNSLYLQMNSLRAEDTA | |
| | | LYYCAKDIAYSRGYFQHWGQGTLVTVSS | |
| Ab- | VL | EIVLTQSPATLSVSPGERATLSCRASQSVSSNLAWYQQKPGQAPRLLI | 115 |
| 012 | | YGASSRATGIPDRFSGSGSGTDFTLTISRLEPEDFAVYYCQQYGSSQIT | |
| | | FGQGTRLEIK | |
| Ab- | HC | EVQLVESGGGLVQPGRSLRLSCTTSGFTFADYAMHWVRQTPGQGLE | 118 |
| 012 | | WVSGISWNGGTMGYADSVKGRFTISRDIAKNSLYLQMNSLRAEDTA | |
| | | LYYCAKDIAYSRGYFQHWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKRVEPKSCDKTHTCPPCP | |
| | | APELLGGPSVFLFPPKPKDTLMISRTPEVTCVVVDVSHEDPEVKFNW | |
| | | YVDGVEVHNAKTKPREEQYNSTYRVVSVLTVLHQDWLNGKEYKC | |
| | | KVSNKALPAPIEKTISKAKGQPREPQVYTLPPSREEMTKNQVSLTCLV | |
| | | KGFYPSDIAVEWESNGQPENNYKTTPPVLDSDGSFFLYSKLTVDKSR | |
| | | WQQGNVFSCSVMHEALHNHYTQKSLSLSPG | |
| Ab- | LC | EIVLTQSPATLSVSPGERATLSCRASQSVSSNLAWYQQKPGQAPRLLI | 119 |
| 012 | | YGASSRATGIPDRFSGSGSGTDFTLTISRLEPEDFAVYYCQQYGSSQIT | |
| | | FGQGTRLEIKRTVAAPSVFIFPPSDEQLKSGTASVVCLLNNFYPREAK | |
| | | VQWKVDNALQSGNSQESVTEQDSKDSTYSLSSTLTLSKADYEKHKV | |
| | | YACEVTHQGLSSPVTKSFNRGEC | |
| Ab- | VH | EVQLVESGGGLVQPGRSLRLSCTTSGFTFADYAMHWVRQTPGKGLE | 122 |
| 014 | | WVSGISWNGVTIDYADSVKGRFTISRDIAKNSLYLQMNSLRAEDTAL | |
| | | YYCAKDIAYSRGYFQHWGQGTLVTVSS | |
| Ab- | VL | EIVLTQSPGTLSLSPGERATLSCRASQSVSSSYLAWYQQKPGQAPRLL | 123 |
| 014 | | IYGASSRATGIPDRESGSGSGTDFTLTISRLEPEDFAVYYCQQYGSSPL | |
| | | TFGGGTKVDIK | |
| Ab- | HC | EVQLVESGGGLVQPGRSLRLSCTTSGFTFADYAMHWVRQTPGKGLE | 126 |
| 014 | | WVSGISWNGVTIDYADSVKGRFTISRDIAKNSLYLQMNSLRAEDTAL | |
| | | YYCAKDIAYSRGYFQHWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKRVEPKSCDKTHTCPPCPA | |
| | | PELLGGPSVFLFPPKPKDTLMISRTPEVTCVVVDVSHEDPEVKENWY | |
| | | VDGVEVHNAKTKPREEQYNSTYRVVSVLTVLHQDWLNGKEYKCK | |
| | | VSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRW | |
| | | QQGNVFSCSVMHEALHNHYTQKSLSLSPG | |
| Ab- | LC | EIVLTQSPGTLSLSPGERATLSCRASQSVSSSYLAWYQQKPGQAPRLL | 127 |
| 014 | | IYGASSRATGIPDRFSGSGSGTDFTLTISRLEPEDFAVYYCQQYGSSPL | |
| | | TFGGGTKVDIKRTVAAPSVFIFPPSDEQLKSGTASVVCLLNNFYPREA | |
| | | KVQWKVDNALQSGNSQESVTEQDSKDSTYSLSSTLTLSKADYEKHK | |
| | | VYACEVTHQGLSSPVTKSFNRGEC | |
| Ab- | VH | EVQLVESGGGLVQPGRSLRLSCTTSGFTFADYAMHWVRQTPGKGLE | 131 |
| 016 | | WVSGISWNGGTIGYADSVKGRFTISRDIAKNSLYLQMNSLRAEDTAL | |
| | | YYCAKDIAYSRGYFQHWGQGTLVTVSS | |
| Ab- | VL | EIVLTQSPGTLSLSPGERATLSCRASQSVSSSYLAWYQQKPGQAPRLL | 132 |
| 016 | | IYGASSRATGIPDRFSGSGSGTDFTLTISRLEPEDFAVYYCQQYGSSPH | |
| | | TFGQGTKVEIK | |
| Ab- | HC | EVQLVESGGGLVQPGRSLRLSCTTSGFTFADYAMHWVRQTPGKGLE | 135 |
| 016 | | WVSGISWNGGTIGYADSVKGRFTISRDIAKNSLYLQMNSLRAEDTAL | |
| | | YYCAKDIAYSRGYFQHWGQGTLVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKRVEPKSCDKTHTCPPCPA | |
| | | PELLGGPSVFLFPPKPKDTLMISRTPEVTCVVVDVSHEDPEVKFNWY | |
| | | VDGVEVHNAKTKPREEQYNSTYRVVSVLTVLHQDWLNGKEYKCK | |
| | | VSNKALPAPIEKTISKAKGQPREPQVYTLPPSREEMTKNQVSLTCLVK | |
| | | GFYPSDIAVEWESNGQPENNYKTTPPVLDSDGSFFLYSKLTVDKSRW | |
| | | QQGNVFSCSVMHEALHNHYTQKSLSLSPG | |
| Ab- | LC | EIVLTQSPGTLSLSPGERATLSCRASQSVSSSYLAWYQQKPGQAPRLL | 136 |
| 016 | | IYGASSRATGIPDRFSGSGSGTDFTLTISRLEPEDFAVYYCQQYGSSPH | |
| | | TFGQGTKVEIKRTVAAPSVFIFPPSDEQLKSGTASVVCLLNNFYPREA | |
| | | KVQWKVDNALQSGNSQESVTEQDSKDSTYSLSSTLTLSKADYEKHK | |
| | | VYACEVTHQGLSSPVTKSFNRGEC | |
| Ab- | VH | EVQLVESGGGLVQPGRSLRLSCTTSGFTFADYAMHWVRQTPGQGLE | 139 |
| 017 | | WVSGISWNGGTMGYADSVKGRFTISRDIAKNSLYLQMNSLRAEDTA | |
| | | LYYCAKDIAYSRGYFQHWGQGTLVTVSS | |
| Ab- | VL | EIVLTQSPGTLSLSPGERATLSCRASQSVSSSYLAWYQQKPGQAPRLL | 140 |
| 017 | | IYGASSRATGIPDRFSGSGSGTDFTLTISRLEPEDFAVYYCQQYGSSPL | |
| | | TFGGGTKVDIK | |
| Ab- | HC | EVQLVESGGGLVQPGRSLRLSCTTSGFTFADYAMHWVRQTPGQGLE | 143 |
| 017 | | WVSGISWNGGTMGYADSVKGRFTISRDIAKNSLYLQMNSLRAEDTA | |
| | | LYYCAKDIAYSRGYFQHWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKRVEPKSCDKTHTCPPCP | |
| | | APELLGGPSVFLFPPKPKDTLMISRTPEVTCVVVDVSHEDPEVKFNW | |
| | | YVDGVEVHNAKTKPREEQYNSTYRVVSVLTVLHQDWLNGKEYKC | |
| | | KVSNKALPAPIEKTISKAKGQPREPQVYTLPPSREEMTKNQVSLTCLV | |
| | | KGFYPSDIAVEWESNGQPENNYKTTPPVLDSDGSFFLYSKLTVDKSR | |
| | | WQQGNVFSCSVMHEALHNHYTQKSLSLSPG | |
| Ab- | LC | EIVLTQSPGTLSLSPGERATLSCRASQSVSSSYLAWYQQKPGQAPRLL | 144 |
| 017 | | IYGASSRATGIPDRFSGSGSGTDFTLTISRLEPEDFAVYYCQQYGSSPL | |
| | | TFGGGTKVDIKRTVAAPSVFIFPPSDEQLKSGTASVVCLLNNFYPREA | |
| | | KVQWKVDNALQSGNSQESVTEQDSKDSTYSLSSTLTLSKADYEKHK | |
| | | VYACEVTHQGLSSPVTKSFNRGEC | |

TABLE 4
Nucleotide Sequences Encoding VH, VL, HC, LC

| | | | SEQ |
|---|---|---|---|
| | | | ID |
| Ab ID | Name | Sequence | NO |
| Ab- | VH | GAAGTGCAGCTGGTGGAGTCTGGGGGAGGCTTGGTACAGCCTGG | 9 |
| 001 | | GGGGTCCCTGAGACTCTCCTGTGCAGCCTCTGGATTCACCTCTCTT | |
| | | GATTATGCCATGCACTGGGTCCGGCAAGCTCCAGGGAAGGGCCT | |
| | | GGAGTGGGTCTCATATATTAGTTGGAATAATAATAACATAGGCTA | |
| | | TGCGGACTCTGTGAAGGGCCGATTCACCATCTCCAGAGACAACGC | |
| | | CAAGAACTCCCTGTATCTGCACATGAACAGTCTGAGACCTGAGGA | |
| | | CACGGCCTTCTATTACTGTGCAAAAGATATCGCATATAACACCGG | |
| | | CTACTTTGACTACTGGGGCCAGGGAACCCTGGTCACCGTCTCCTC | |
| | | A | |
| Ab- | VL | GAAATTGTGTTGACGCAGTCTCCAGGCACCCTGTCTTTGTCTCCA | 10 |
| 001 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCTCTCACTTTCGGCGGAGGGACCAAGGT | |
| | | GGAAATCAAA | |
| Ab- | HC | GAAGTGCAGCTGGTGGAGTCTGGGGGAGGCTTGGTACAGCCTGG | 13 |
| 001 | | GGGGTCCCTGAGACTCTCCTGTGCAGCCTCTGGATTCACCTCTCTT | |
| | | GATTATGCCATGCACTGGGTCCGGCAAGCTCCAGGGAAGGGCCT | |
| | | GGAGTGGGTCTCATATATTAGTTGGAATAATAATAACATAGGCTA | |
| | | TGCGGACTCTGTGAAGGGCCGATTCACCATCTCCAGAGACAACGC | |
| | | CAAGAACTCCCTGTATCTGCACATGAACAGTCTGAGACCTGAGGA | |
| | | CACGGCCTTCTATTACTGTGCAAAAGATATCGCATATAACACCGG | |
| | | CTACTTTGACTACTGGGGCCAGGGAACCCTGGTCACCGTCTCCTC | |
| | | AGCTAGCACTAAAGGGCCTTCTGTATTTCCCTTGGCCCCGTCCAG | |
| | | CAAATCGACCTCGGGAGGGACAGCCGCCCTGGGTTGCCTTGTGAA | |
| | | AGATTATTTCCCTGAGCCAGTTACCGTAAGTTGGAACAGTGGGGC | |
| | | GCTGACAAGTGGTGTGCACACGTTTCCTGCCGTCCTGCAATCATC | |
| | | GGGCTTGTATAGCCTCAGCTCTGTGGTCACTGTCCCAAGTTCATC | |
| | | GCTGGGCACTCAGACGTATATTTGCAATGTGAACCACAAACCTTC | |
| | | AAATACAAAAGTGGATAAACGCGTAGAACCGAAATCGTGTGATA | |
| | | AAACTCACACATGCCCGCCATGCCCGGCACCTGAACTGCTTGGTG | |
| | | GTCCCAGCGTGTTCCTGTTCCCGCCGAAGCCTAAAGATACTCTAA | |
| | | TGATCAGCCGTACGCCAGAGGTGACATGTGTCGTGGTTGACGTGT | |
| | | CCCACGAAGATCCCGAAGTTAAGTTCAATTGGTATGTTGATGGTG | |
| | | TAGAGGTACACAATGCTAAGACTAAACCTCGCGAGGAGCAGTAC | |
| | | AATTCGACCTATCGTGTCGTGAGCGTTCTGACCGTCCTTCACCAA | |
| | | GATTGGCTTAACGGCAAAGAATATAAGTGCAAGGTAAGCAATAA | |
| | | AGCACTTCCGGCCCCAATCGAGAAAACCATTTCCAAGGCCAAAG | |
| | | GTCAACCAAGAGAACCCCAGGTGTATACTCTTCCGCCTTCTCGTG | |
| | | AGGAAATGACTAAAAATCAAGTATCCCTTACGTGTCTGGTTAAAG | |
| | | GTTTTTATCCTAGCGATATTGCTGTTGAATGGGAATCGAACGGTC | |
| | | AGCCGGAGAATAATTATAAAACAACGCCACCCGTCCTGGATAGC | |
| | | GACGGCTCATTTTTTCTGTATAGCAAACTGACTGTAGATAAATCA | |
| | | CGGTGGCAGCAGGGCAATGTATTCAGTTGCTCCGTTATGCATGAA | |
| | | GCGTTACATAATCACTACACGCAGAAATCTCTTAGTCTTTCACCC | |
| | | GGT | |
| Ab- | LC | GAAATTGTGTTGACGCAGTCTCCAGGCACCCTGTCTTTGTCTCCA | 14 |
| 001 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCTCTCACTTTCGGCGGAGGGACCAAGGT | |
| | | GGAAATCAAACGTACGGTAGCTGCCCCTTCAGTTTTTATCTTTCCG | |
| | | CCGTCTGACGAGCAGTTAAAATCCGGGACCGCTTCTGTAGTTTGC | |
| | | CTGCTGAATAATTTTTATCCGCGTGAGGCTAAAGTACAATGGAAA | |
| | | GTCGACAATGCTTTGCAGTCGGGAAATTCACAGGAAAGTGTTACG | |
| | | GAGCAGGATTCTAAAGATTCCACATATTCACTCAGCTCCACCCTT | |
| | | ACACTGAGCAAAGCCGACTATGAAAAACATAAAGTTTACGCATG | |
| | | TGAGGTGACGCACCAAGGATTATCCAGTCCGGTCACAAAATCGTT | |
| | | TAACCGCGGTGAGTGT | |
| Ab- | VH | GAAGTGCAGCTGGTGGAGTCTGGGGGAGGCTTGGTACAGCCTGG | 18 |
| 002 | | GGGGTCCCTGAGACTCTCCTGTGCAGCCTCTGGATTCACCTCTGTT | |
| | | GATTATGCCATGCACTGGGTCCGGCAAGCTCCAGGGAAGGGCCT | |
| | | AGAGTGGGTCTCATATATTAGTTGGAATAATAATAACATAGGCTA | |
| | | TGCGGACTCTGTGAAGGGCCGATTCACCATCTCCAGAGACAACGC | |
| | | CAAGAACTCCCTCTATCTGCACATGAGCAGTCTGAGAGCTGAGGA | |
| | | CACGGCCTTGTATTATTGTGCAAAAGATATCGCATATAACACCGG | |
| | | GTACTTTGACTACTGGGGCCAGGGAACCCTGGTCACCGTCTCCTC | |
| | | A | |
| Ab- | VL | GAAATTGTGTTGACGCAGTCTCCAGGCACCCTGTCTTTGTCTCCA | 19 |
| 002 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCATTCACTTTCGGCCCTGGGACCAAGGT | |
| | | GGAAATCAAA | |
| Ab- | HC | GAAGTGCAGCTGGTGGAGTCTGGGGGAGGCTTGGTACAGCCTGG | 22 |
| 002 | | GGGGTCCCTGAGACTCTCCTGTGCAGCCTCTGGATTCACCTCTGTT | |
| | | GATTATGCCATGCACTGGGTCCGGCAAGCTCCAGGGAAGGGCCT | |
| | | AGAGTGGGTCTCATATATTAGTTGGAATAATAATAACATAGGCTA | |
| | | TGCGGACTCTGTGAAGGGCCGATTCACCATCTCCAGAGACAACGC | |
| | | CAAGAACTCCCTCTATCTGCACATGAGCAGTCTGAGAGCTGAGGA | |
| | | CACGGCCTTGTATTATTGTGCAAAAGATATCGCATATAACACCGG | |
| | | GTACTTTGACTACTGGGGCCAGGGAACCCTGGTCACCGTCTCCTC | |
| | | AGCTAGCACTAAAGGGCCTTCTGTATTTCCCTTGGCCCCGTCCAG | |
| | | CAAATCGACCTCGGGAGGGACAGCCGCCCTGGGTTGCCTTGTGAA | |
| | | AGATTATTTCCCTGAGCCAGTTACCGTAAGTTGGAACAGTGGGGC | |
| | | GCTGACAAGTGGTGTGCACACGTTTCCTGCCGTCCTGCAATCATC | |
| | | GGGCTTGTATAGCCTCAGCTCTGTGGTCACTGTCCCAAGTTCATC | |
| | | GCTGGGCACTCAGACGTATATTTGCAATGTGAACCACAAACCTTC | |
| | | AAATACAAAAGTGGATAAACGCGTAGAACCGAAATCGTGTGATA | |
| | | AAACTCACACATGCCCGCCATGCCCGGCACCTGAACTGCTTGGTG | |
| | | GTCCCAGCGTGTTCCTGTTCCCGCCGAAGCCTAAAGATACTCTAA | |
| | | TGATCAGCCGTACGCCAGAGGTGACATGTGTCGTGGTTGACGTGT | |
| | | CCCACGAAGATCCCGAAGTTAAGTTCAATTGGTATGTTGATGGTG | |
| | | TAGAGGTACACAATGCTAAGACTAAACCTCGCGAGGAGCAGTAC | |
| | | AATTCGACCTATCGTGTCGTGAGCGTTCTGACCGTCCTTCACCAA | |
| | | GATTGGCTTAACGGCAAAGAATATAAGTGCAAGGTAAGCAATAA | |
| | | AGCACTTCCGGCCCCAATCGAGAAAACCATTTCCAAGGCCAAAG | |
| | | GTCAACCAAGAGAACCCCAGGTGTATACTCTTCCGCCTTCTCGTG | |
| | | AGGAAATGACTAAAAATCAAGTATCCCTTACGTGTCTGGTTAAAG | |
| | | GTTTTTATCCTAGCGATATTGCTGTTGAATGGGAATCGAACGGTC | |
| | | AGCCGGAGAATAATTATAAAACAACGCCACCCGTCCTGGATAGC | |
| | | GACGGCTCATTTTTTCTGTATAGCAAACTGACTGTAGATAAATCA | |
| | | CGGTGGCAGCAGGGCAATGTATTCAGTTGCTCCGTTATGCATGAA | |
| | | GCGTTACATAATCACTACACGCAGAAATCTCTTAGTCTTTCACCC | |
| | | GGT | |
| Ab- | LC | GAAATTGTGTTGACGCAGTCTCCAGGCACCCTGTCTTTGTCTCCA | 23 |
| 002 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCATTCACTTTCGGCCCTGGGACCAAGGT | |
| | | GGAAATCAAACGTACGGTAGCTGCCCCTTCAGTTTTTATCTTTCCG | |
| | | CCGTCTGACGAGCAGTTAAAATCCGGGACCGCTTCTGTAGTTTGC | |
| | | CTGCTGAATAATTTTTATCCGCGTGAGGCTAAAGTACAATGGAAA | |
| | | GTCGACAATGCTTTGCAGTCGGGAAATTCACAGGAAAGTGTTACG | |
| | | GAGCAGGATTCTAAAGATTCCACATATTCACTCAGCTCCACCCTT | |
| | | ACACTGAGCAAAGCCGACTATGAAAAACATAAAGTTTACGCATG | |
| | | TGAGGTGACGCACCAAGGATTATCCAGTCCGGTCACAAAATCGTT | |
| | | TAACCGCGGTGAGTGT | |
| Ab- | VH | GAAGTGCAGCTGGTGGAGTCTGGGGGAGGCTTGGTACAGCCTGG | 29 |
| 003 | | CAGGTCCCTGAGACTCTCCTGTACAACCTCTGGATTCACCTTTGCT | |
| | | GATTATGCCATGCACTGGGTCCGGCAAACTCCAGGGCAGGGCCTG | |
| | | GAGTGGGTCTCAGGTATTAGTTGGAATGGTGGTACCATGGGCTAT | |
| | | GCGGACTCTGTGAAGGGCCGATTCACCATCTCCAGAGACATCGCC | |
| | | AAGAACTCCCTGTATCTGCAAATGAACAGTCTGAGAGCTGAGGA | |
| | | CACGGCCTTATATTATTGTGCAAAGGATATCGCATATAGCAGAGG | |
| | | ATACTTCCAGCACTGGGGCCAGGGCACCCTGGTCACCGTCTCTTC | |
| | | A | |
| Ab- | VL | GAAATTGTGTTGACACAGTCTCCAGGCACCCTGTCTTTGTCTCCA | 30 |
| 003 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCTCGGACGTTCGGCCAAGGGACCAAGG | |
| | | TGGAAATCAAA | |
| Ab- | HC | GAAGTGCAGCTGGTGGAGTCTGGGGGAGGCTTGGTACAGCCTGG | 33 |
| 003 | | CAGGTCCCTGAGACTCTCCTGTACAACCTCTGGATTCACCTTTGCT | |
| | | GATTATGCCATGCACTGGGTCCGGCAAACTCCAGGGCAGGGCCTG | |
| | | GAGTGGGTCTCAGGTATTAGTTGGAATGGTGGTACCATGGGCTAT | |
| | | GCGGACTCTGTGAAGGGCCGATTCACCATCTCCAGAGACATCGCC | |
| | | AAGAACTCCCTGTATCTGCAAATGAACAGTCTGAGAGCTGAGGA | |
| | | CACGGCCTTATATTATTGTGCAAAGGATATCGCATATAGCAGAGG | |
| | | ATACTTCCAGCACTGGGGCCAGGGCACCCTGGTCACCGTCTCTTC | |
| | | AGCTAGCACTAAAGGGCCTTCTGTATTTCCCTTGGCCCCGTCCAG | |
| | | CAAATCGACCTCGGGAGGGACAGCCGCCCTGGGTTGCCTTGTGAA | |
| | | AGATTATTTCCCTGAGCCAGTTACCGTAAGTTGGAACAGTGGGGC | |
| | | GCTGACAAGTGGTGTGCACACGTTTCCTGCCGTCCTGCAATCATC | |
| | | GGGCTTGTATAGCCTCAGCTCTGTGGTCACTGTCCCAAGTTCATC | |
| | | GCTGGGCACTCAGACGTATATTTGCAATGTGAACCACAAACCTTC | |
| | | AAATACAAAAGTGGATAAACGCGTAGAACCGAAATCGTGTGATA | |
| | | AAACTCACACATGCCCGCCATGCCCGGCACCTGAACTGCTTGGTG | |
| | | GTCCCAGCGTGTTCCTGTTCCCGCCGAAGCCTAAAGATACTCTAA | |
| | | TGATCAGCCGTACGCCAGAGGTGACATGTGTCGTGGTTGACGTGT | |
| | | CCCACGAAGATCCCGAAGTTAAGTTCAATTGGTATGTTGATGGTG | |
| | | TAGAGGTACACAATGCTAAGACTAAACCTCGCGAGGAGCAGTAC | |
| | | AATTCGACCTATCGTGTCGTGAGCGTTCTGACCGTCCTTCACCAA | |
| | | GATTGGCTTAACGGCAAAGAATATAAGTGCAAGGTAAGCAATAA | |
| | | AGCACTTCCGGCCCCAATCGAGAAAACCATTTCCAAGGCCAAAG | |
| | | GTCAACCAAGAGAACCCCAGGTGTATACTCTTCCGCCTTCTCGTG | |
| | | AGGAAATGACTAAAAATCAAGTATCCCTTACGTGTCTGGTTAAAG | |
| | | GTTTTTATCCTAGCGATATTGCTGTTGAATGGGAATCGAACGGTC | |
| | | AGCCGGAGAATAATTATAAAACAACGCCACCCGTCCTGGATAGC | |
| | | GACGGCTCATTTTTTCTGTATAGCAAACTGACTGTAGATAAATCA | |
| | | CGGTGGCAGCAGGGCAATGTATTCAGTTGCTCCGTTATGCATGAA | |
| | | GCGTTACATAATCACTACACGCAGAAATCTCTTAGTCTTTCACCC | |
| | | GGT | |
| Ab- | LC | GAAATTGTGTTGACACAGTCTCCAGGCACCCTGTCTTTGTCTCCA | 34 |
| 003 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCTCGGACGTTCGGCCAAGGGACCAAGG | |
| | | TGGAAATCAAACGTACGGTAGCTGCCCCTTCAGTTTTTATCTTTCC | |
| | | GCCGTCTGACGAGCAGTTAAAATCCGGGACCGCTTCTGTAGTTTG | |
| | | CCTGCTGAATAATTTTTATCCGCGTGAGGCTAAAGTACAATGGAA | |
| | | AGTCGACAATGCTTTGCAGTCGGGAAATTCACAGGAAAGTGTTAC | |
| | | GGAGCAGGATTCTAAAGATTCCACATATTCACTCAGCTCCACCCT | |
| | | TACACTGAGCAAAGCCGACTATGAAAAACATAAAGTTTACGCAT | |
| | | GTGAGGTGACGCACCAAGGATTATCCAGTCCGGTCACAAAATCGT | |
| | | TTAACCGCGGTGAGTGT | |
| Ab- | VH | GAAGTGCAGCTGGTGGAGTCTGGGGGAGGCTTGGTACAGCCTGG | 38 |
| 004 | | GGGGTCCCTGAGACTCTCCTGTACAACCTCTGGATTCACCTTTGCT | |
| | | GATTATGCCATGCACTGGGTCCGGCAAACTCCAGGGAAGGGCCT | |
| | | GGAGTGGGTCTCAGGTATTAGTTGGAATGGTGTTACCATTGACTA | |
| | | TGCGGACTCTGTGAAGGGCCGATTCACCATCTCCAGAGACATCGC | |
| | | CAAGAACTCCCTGTATCTGCAAATGAACAGTCTGAGAGCTGAGG | |
| | | ACACGGCCTTATATTATTGTGCAAAGGATATAGCATATAGCAGAG | |
| | | GATACTTCCAGCACTGGGGCCAGGGCACCCTGGTCACCGTCTCCT | |
| | | CA | |
| Ab- | VL | GAAATTGTGTTGACGCAGTCTCCAGCCACCCTGTCTGTGTCTCCA | 39 |
| 004 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCTCGGACGTTCGGCCAAGGGACCAAGG | |
| | | TGGAGATCAAA | |
| Ab- | HC | GAAGTGCAGCTGGTGGAGTCTGGGGGAGGCTTGGTACAGCCTGG | 42 |
| 004 | | GGGGTCCCTGAGACTCTCCTGTACAACCTCTGGATTCACCTTTGCT | |
| | | GATTATGCCATGCACTGGGTCCGGCAAACTCCAGGGAAGGGCCT | |
| | | GGAGTGGGTCTCAGGTATTAGTTGGAATGGTGTTACCATTGACTA | |
| | | TGCGGACTCTGTGAAGGGCCGATTCACCATCTCCAGAGACATCGC | |
| | | CAAGAACTCCCTGTATCTGCAAATGAACAGTCTGAGAGCTGAGG | |
| | | ACACGGCCTTATATTATTGTGCAAAGGATATAGCATATAGCAGAG | |
| | | GATACTTCCAGCACTGGGGCCAGGGCACCCTGGTCACCGTCTCCT | |
| | | CAGCTAGCACTAAAGGGCCTTCTGTATTTCCCTTGGCCCCGTCCA | |
| | | GCAAATCGACCTCGGGAGGGACAGCCGCCCTGGGTTGCCTTGTGA | |
| | | AAGATTATTTCCCTGAGCCAGTTACCGTAAGTTGGAACAGTGGGG | |
| | | CGCTGACAAGTGGTGTGCACACGTTTCCTGCCGTCCTGCAATCAT | |
| | | CGGGCTTGTATAGCCTCAGCTCTGTGGTCACTGTCCCAAGTTCATC | |
| | | GCTGGGCACTCAGACGTATATTTGCAATGTGAACCACAAACCTTC | |
| | | AAATACAAAAGTGGATAAACGCGTAGAACCGAAATCGTGTGATA | |
| | | AAACTCACACATGCCCGCCATGCCCGGCACCTGAACTGCTTGGTG | |
| | | GTCCCAGCGTGTTCCTGTTCCCGCCGAAGCCTAAAGATACTCTAA | |
| | | TGATCAGCCGTACGCCAGAGGTGACATGTGTCGTGGTTGACGTGT | |
| | | CCCACGAAGATCCCGAAGTTAAGTTCAATTGGTATGTTGATGGTG | |
| | | TAGAGGTACACAATGCTAAGACTAAACCTCGCGAGGAGCAGTAC | |
| | | AATTCGACCTATCGTGTCGTGAGCGTTCTGACCGTCCTTCACCAA | |
| | | GATTGGCTTAACGGCAAAGAATATAAGTGCAAGGTAAGCAATAA | |
| | | AGCACTTCCGGCCCCAATCGAGAAAACCATTTCCAAGGCCAAAG | |
| | | GTCAACCAAGAGAACCCCAGGTGTATACTCTTCCGCCTTCTCGTG | |
| | | AGGAAATGACTAAAAATCAAGTATCCCTTACGTGTCTGGTTAAAG | |
| | | GTTTTTATCCTAGCGATATTGCTGTTGAATGGGAATCGAACGGTC | |
| | | AGCCGGAGAATAATTATAAAACAACGCCACCCGTCCTGGATAGC | |
| | | GACGGCTCATTTTTTCTGTATAGCAAACTGACTGTAGATAAATCA | |
| | | CGGTGGCAGCAGGGCAATGTATTCAGTTGCTCCGTTATGCATGAA | |
| | | GCGTTACATAATCACTACACGCAGAAATCTCTTAGTCTTTCACCC | |
| | | GGT | |
| Ab- | LC | GAAATTGTGTTGACGCAGTCTCCAGCCACCCTGTCTGTGTCTCCA | 43 |
| 004 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCTCGGACGTTCGGCCAAGGGACCAAGG | |
| | | TGGAGATCAAACGTACGGTAGCTGCCCCTTCAGTTTTTATCTTTCC | |
| | | GCCGTCTGACGAGCAGTTAAAATCCGGGACCGCTTCTGTAGTTTG | |
| | | CCTGCTGAATAATTTTTATCCGCGTGAGGCTAAAGTACAATGGAA | |
| | | AGTCGACAATGCTTTGCAGTCGGGAAATTCACAGGAAAGTGTTAC | |
| | | GGAGCAGGATTCTAAAGATTCCACATATTCACTCAGCTCCACCCT | |
| | | TACACTGAGCAAAGCCGACTATGAAAAACATAAAGTTTACGCAT | |
| | | GTGAGGTGACGCACCAAGGATTATCCAGTCCGGTCACAAAATCGT | |
| | | TTAACCGCGGTGAGTGT | |
| Ab- | VH | GAAGTGCAGCTGGTGGAGTCTGGGGGAGGCTTGGTACAGCCTGG | 48 |
| 005 | | CGGGTCCCTGAGACTCTCCTGTACAACCTCTGGATTCACCTTTGCT | |
| | | GATTATGCCATGCACTGGGTCCGGCAAACTCCAGGGAAGGGCCT | |
| | | GGAGTGGGTCTCAGGTATTAGTTGGAATGGTGGTACCATAGGCTA | |
| | | TGCGGACTCTGTGAAGGGCCGATTCACCATCTCCAGAGACATCGC | |
| | | CAAGAACTCCCTGTATCTGCAAATGAACAGTCTGAGAGCTGAGG | |
| | | ACACGGCCTTATATTATTGTGCAAAGGATATAGCATATAGCAGAG | |
| | | GGTACTTTCAGCACTGGGGCCAGGGCACCCTGGTCACCGTCTCCT | |
| | | CA | |
| Ab- | VL | GAAATTGTGTTGACACAGTCTCCAGGCACCCTGTCTTTGTCTCCA | 49 |
| 005 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCGTACACTTTTGGCCAGGGGACCAAAGT | |
| | | GGATATCAAA | |
| Ab- | HC | GAAGTGCAGCTGGTGGAGTCTGGGGGAGGCTTGGTACAGCCTGG | 52 |
| 005 | | CGGGTCCCTGAGACTCTCCTGTACAACCTCTGGATTCACCTTTGCT | |
| | | GATTATGCCATGCACTGGGTCCGGCAAACTCCAGGGAAGGGCCT | |
| | | GGAGTGGGTCTCAGGTATTAGTTGGAATGGTGGTACCATAGGCTA | |
| | | TGCGGACTCTGTGAAGGGCCGATTCACCATCTCCAGAGACATCGC | |
| | | CAAGAACTCCCTGTATCTGCAAATGAACAGTCTGAGAGCTGAGG | |
| | | ACACGGCCTTATATTATTGTGCAAAGGATATAGCATATAGCAGAG | |
| | | GGTACTTTCAGCACTGGGGCCAGGGCACCCTGGTCACCGTCTCCT | |
| | | CAGCTAGCACTAAAGGGCCTTCTGTATTTCCCTTGGCCCCGTCCA | |
| | | GCAAATCGACCTCGGGAGGGACAGCCGCCCTGGGTTGCCTTGTGA | |
| | | AAGATTATTTCCCTGAGCCAGTTACCGTAAGTTGGAACAGTGGGG | |
| | | CGCTGACAAGTGGTGTGCACACGTTTCCTGCCGTCCTGCAATCAT | |
| | | CGGGCTTGTATAGCCTCAGCTCTGTGGTCACTGTCCCAAGTTCATC | |
| | | GCTGGGCACTCAGACGTATATTTGCAATGTGAACCACAAACCTTC | |
| | | AAATACAAAAGTGGATAAACGCGTAGAACCGAAATCGTGTGATA | |
| | | AAACTCACACATGCCCGCCATGCCCGGCACCTGAACTGCTTGGTG | |
| | | GTCCCAGCGTGTTCCTGTTCCCGCCGAAGCCTAAAGATACTCTAA | |
| | | TGATCAGCCGTACGCCAGAGGTGACATGTGTCGTGGTTGACGTGT | |
| | | CCCACGAAGATCCCGAAGTTAAGTTCAATTGGTATGTTGATGGTG | |
| | | TAGAGGTACACAATGCTAAGACTAAACCTCGCGAGGAGCAGTAC | |
| | | AATTCGACCTATCGTGTCGTGAGCGTTCTGACCGTCCTTCACCAA | |
| | | GATTGGCTTAACGGCAAAGAATATAAGTGCAAGGTAAGCAATAA | |
| | | AGCACTTCCGGCCCCAATCGAGAAAACCATTTCCAAGGCCAAAG | |
| | | GTCAACCAAGAGAACCCCAGGTGTATACTCTTCCGCCTTCTCGTG | |
| | | AGGAAATGACTAAAAATCAAGTATCCCTTACGTGTCTGGTTAAAG | |
| | | GTTTTTATCCTAGCGATATTGCTGTTGAATGGGAATCGAACGGTC | |
| | | AGCCGGAGAATAATTATAAAACAACGCCACCCGTCCTGGATAGC | |
| | | GACGGCTCATTTTTTCTGTATAGCAAACTGACTGTAGATAAATCA | |
| | | CGGTGGCAGCAGGGCAATGTATTCAGTTGCTCCGTTATGCATGAA | |
| | | GCGTTACATAATCACTACACGCAGAAATCTCTTAGTCTTTCACCC | |
| | | GGT | |
| Ab- | LC | GAAATTGTGTTGACACAGTCTCCAGGCACCCTGTCTTTGTCTCCA | 53 |
| 005 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCGTACACTTTTGGCCAGGGGACCAAAGT | |
| | | GGATATCAAACGTACGGTAGCTGCCCCTTCAGTTTTTATCTTTCCG | |
| | | CCGTCTGACGAGCAGTTAAAATCCGGGACCGCTTCTGTAGTTTGC | |
| | | CTGCTGAATAATTTTTATCCGCGTGAGGCTAAAGTACAATGGAAA | |
| | | GTCGACAATGCTTTGCAGTCGGGAAATTCACAGGAAAGTGTTACG | |
| | | GAGCAGGATTCTAAAGATTCCACATATTCACTCAGCTCCACCCTT | |
| | | ACACTGAGCAAAGCCGACTATGAAAAACATAAAGTTTACGCATG | |
| | | TGAGGTGACGCACCAAGGATTATCCAGTCCGGTCACAAAATCGTT | |
| | | TAACCGCGGTGAGTGT | |
| Ab- | VH | GAGGTGCAGCTGGTGGAGTCTGGGGGAGGCCTGGTCAAGCCTGG | 62 |
| 006 | | GGGGTCCCTGAGACTCTCCTGTACAGCCTCTGGATTCACCTTTGAT | |
| | | GATTATGACATGACCTGGGTCCGCCAAGCTCCAGGGAAGGGGCT | |
| | | GGAGTGGGTCTCTGGTATTAATTGGAATGGTGGTAACACAGATTA | |
| | | TGCAGACTCTGTGAAGGGCCGATTCACCATCTCCAGAGACAACAC | |
| | | CAAGAACTCCCTGTATCTGCAGATGAACAGTCTGAGAGCCGAGG | |
| | | ACACGGCCTTGTATTACTGTGCGAGAAAAGGGAGGGATATTGTA | |
| | | GTAGTAAAAGGATTTTACTACCACTACTACTACATGGACGTCTGG | |
| | | GGCAAAGGGATCACGGTCACTGTCTCCTCA | |
| Ab- | VL | GATATTGTGATGACCCAGTCTCCACTCTCCCTGCCCGTCACCCCTG | 63 |
| 006 | | GAGAGCCGGCCTCCATCTCCTGCAGGTCTAGTCAGAGCCTCCTGC | |
| | | ATAGTAATGGATACAACTATTTGGATTGGTACCTGCAGAAGCCAG | |
| | | GGCAGTCTCCACAGCTCCTGATCTATTTGGGTTCTAATCGGGCCTC | |
| | | CGGGGTCCCTGACAGGTTCAGTGGCAGTGGATCAGGCACAGATTT | |
| | | TACACTGAAAATCAGCAGAGTGGAGGCTGAGGATGTTGGGGTTT | |
| | | ATTACTGCATGCAAGCTCTACAAACTCCGCTCACTTTCGGCGGAG | |
| | | GGACCAAAGTGGATATCAAA | |
| Ab- | HC | GAGGTGCAGCTGGTGGAGTCTGGGGGAGGCCTGGTCAAGCCTGG | 66 |
| 006 | | GGGGTCCCTGAGACTCTCCTGTACAGCCTCTGGATTCACCTTTGAT | |
| | | GATTATGACATGACCTGGGTCCGCCAAGCTCCAGGGAAGGGGCT | |
| | | GGAGTGGGTCTCTGGTATTAATTGGAATGGTGGTAACACAGATTA | |
| | | TGCAGACTCTGTGAAGGGCCGATTCACCATCTCCAGAGACAACAC | |
| | | CAAGAACTCCCTGTATCTGCAGATGAACAGTCTGAGAGCCGAGG | |
| | | ACACGGCCTTGTATTACTGTGCGAGAAAAGGGAGGGATATTGTA | |
| | | GTAGTAAAAGGATTTTACTACCACTACTACTACATGGACGTCTGG | |
| | | GGCAAAGGGATCACGGTCACTGTCTCCTCAGCTAGCACTAAAGG | |
| | | GCCTTCTGTATTTCCCTTGGCCCCGTCCAGCAAATCGACCTCGGG | |
| | | AGGGACAGCCGCCCTGGGTTGCCTTGTGAAAGATTATTTCCCTGA | |
| | | GCCAGTTACCGTAAGTTGGAACAGTGGGGCGCTGACAAGTGGTG | |
| | | TGCACACGTTTCCTGCCGTCCTGCAATCATCGGGCTTGTATAGCCT | |
| | | CAGCTCTGTGGTCACTGTCCCAAGTTCATCGCTGGGCACTCAGAC | |
| | | GTATATTTGCAATGTGAACCACAAACCTTCAAATACAAAAGTGGA | |
| | | TAAACGCGTAGAACCGAAATCGTGTGATAAAACTCACACATGCC | |
| | | CGCCATGCCCGGCACCTGAACTGCTTGGTGGTCCCAGCGTGTTCC | 67 |
| | | TGTTCCCGCCGAAGCCTAAAGATACTCTAATGATCAGCCGTACGC | |
| | | CAGAGGTGACATGTGTCGTGGTTGACGTGTCCCACGAAGATCCCG | |
| | | AAGTTAAGTTCAATTGGTATGTTGATGGTGTAGAGGTACACAATG | |
| | | CTAAGACTAAACCTCGCGAGGAGCAGTACAATTCGACCTATCGTG | |
| | | TCGTGAGCGTTCTGACCGTCCTTCACCAAGATTGGCTTAACGGCA | |
| | | AAGAATATAAGTGCAAGGTAAGCAATAAAGCACTTCCGGCCCCA | |
| | | ATCGAGAAAACCATTTCCAAGGCCAAAGGTCAACCAAGAGAACC | |
| | | CCAGGTGTATACTCTTCCGCCTTCTCGTGAGGAAATGACTAAAAA | |
| | | TCAAGTATCCCTTACGTGTCTGGTTAAAGGTTTTTATCCTAGCGAT | |
| | | ATTGCTGTTGAATGGGAATCGAACGGTCAGCCGGAGAATAATTAT | |
| | | AAAACAACGCCACCCGTCCTGGATAGCGACGGCTCATTTTTTCTG | |
| | | TATAGCAAACTGACTGTAGATAAATCACGGTGGCAGCAGGGCAA | |
| | | TGTATTCAGTTGCTCCGTTATGCATGAAGCGTTACATAATCACTAC | |
| | | ACGCAGAAATCTCTTAGTCTTTCACCCGGT | |
| Ab- | LC | GATATTGTGATGACCCAGTCTCCACTCTCCCTGCCCGTCACCCCTG | |
| 006 | | GAGAGCCGGCCTCCATCTCCTGCAGGTCTAGTCAGAGCCTCCTGC | |
| | | ATAGTAATGGATACAACTATTTGGATTGGTACCTGCAGAAGCCAG | |
| | | GGCAGTCTCCACAGCTCCTGATCTATTTGGGTTCTAATCGGGCCTC | |
| | | CGGGGTCCCTGACAGGTTCAGTGGCAGTGGATCAGGCACAGATTT | |
| | | TACACTGAAAATCAGCAGAGTGGAGGCTGAGGATGTTGGGGTTT | |
| | | ATTACTGCATGCAAGCTCTACAAACTCCGCTCACTTTCGGCGGAG | |
| | | GGACCAAAGTGGATATCAAACGTACGGTAGCTGCCCCTTCAGTTT | |
| | | TTATCTTTCCGCCGTCTGACGAGCAGTTAAAATCCGGGACCGCTT | |
| | | CTGTAGTTTGCCTGCTGAATAATTTTTATCCGCGTGAGGCTAAAGT | |
| | | ACAATGGAAAGTCGACAATGCTTTGCAGTCGGGAAATTCACAGG | |
| | | AAAGTGTTACGGAGCAGGATTCTAAAGATTCCACATATTCACTCA | |
| | | GCTCCACCCTTACACTGAGCAAAGCCGACTATGAAAAACATAAA | |
| | | GTTTACGCATGTGAGGTGACGCACCAAGGATTATCCAGTCCGGTC | |
| | | ACAAAATCGTTTAACCGCGGTGAGTGT | |
| Ab- | VH | GAGGTGCAGCTGGTGGAGTCTGGGGGAGGTGTGGTTCGGCCTGG | 74 |
| 007 | | GGGGTCCCTGAGACTCTCCTGTGCAGCCTCTGGATTCACCTTTGAT | |
| | | GATTATGCCATGAGTTGGGTCCGCCAAGCTCCAGGGAAGGGGCT | |
| | | GGAGTGGGTCTCTGATATTAATTGGAATGGTGGTACCACAAGTTA | |
| | | TGCAGACTCTGTGAAGGGCCGATTCACCATCTCCAGAGACAACGC | |
| | | CAAGAACTCCCTCTTTCTACAAATGAACAGTCTGAGAGCCGAGGA | |
| | | CACGGCCTTGTATTACTGTGCGAGAAAAGGGAGGGATACTGTAGT | |
| | | AGTAAAAGGGTATTATTATTTTTCCTACTACATGGACGTCTGGGG | |
| | | CAAAGGGAGTACGGTCACCGTCTCCTCA | |
| Ab- | VL | GATATTGTGATGACTCAGTCTCCACTCTCCCTGCCCGTCACCCCTG | 75 |
| 007 | | GAGAGCCGGCCTCCATCTCCTGCAGGTCTAGTCAGAGCCTCCTGC | |
| | | ATAGTAATGGATACAACTATTTGGATTGGTACCTGCAGAAGCCAG | |
| | | GGCAGTCTCCACAGCTCCTGATCTATTTGGGTTCTAATCGGGCCTC | |
| | | CGGGGTCCCTGACAGGTTCAGTGGCAGTGGATCAGGCACAGATTT | |
| | | TACACTGAAAATCAGCAGAGTGGAGGCTGAGGATGTTGGGGTTT | |
| | | ATTACTGCATGCAAGCTCTACAAACTCCGTGGACGTTCGGCCAAG | |
| | | GGACCAAGCTGGAAATCAAA | |
| Ab- | HC | GAGGTGCAGCTGGTGGAGTCTGGGGGAGGTGTGGTTCGGCCTGG | 78 |
| 007 | | GGGGTCCCTGAGACTCTCCTGTGCAGCCTCTGGATTCACCTTTGAT | |
| | | GATTATGCCATGAGTTGGGTCCGCCAAGCTCCAGGGAAGGGGCT | |
| | | GGAGTGGGTCTCTGATATTAATTGGAATGGTGGTACCACAAGTTA | |
| | | TGCAGACTCTGTGAAGGGCCGATTCACCATCTCCAGAGACAACGC | |
| | | CAAGAACTCCCTCTTTCTACAAATGAACAGTCTGAGAGCCGAGGA | |
| | | CACGGCCTTGTATTACTGTGCGAGAAAAGGGAGGGATACTGTAGT | |
| | | AGTAAAAGGGTATTATTATTTTTCCTACTACATGGACGTCTGGGG | |
| | | CAAAGGGAGTACGGTCACCGTCTCCTCAGCTAGCACTAAAGGGC | |
| | | CTTCTGTATTTCCCTTGGCCCCGTCCAGCAAATCGACCTCGGGAG | |
| | | GGACAGCCGCCCTGGGTTGCCTTGTGAAAGATTATTTCCCTGAGC | |
| | | CAGTTACCGTAAGTTGGAACAGTGGGGCGCTGACAAGTGGTGTG | |
| | | CACACGTTTCCTGCCGTCCTGCAATCATCGGGCTTGTATAGCCTCA | |
| | | GCTCTGTGGTCACTGTCCCAAGTTCATCGCTGGGCACTCAGACGT | |
| | | ATATTTGCAATGTGAACCACAAACCTTCAAATACAAAAGTGGATA | |
| | | AACGCGTAGAACCGAAATCGTGTGATAAAACTCACACATGCCCG | |
| | | CCATGCCCGGCACCTGAACTGCTTGGTGGTCCCAGCGTGTTCCTG | |
| | | TTCCCGCCGAAGCCTAAAGATACTCTAATGATCAGCCGTACGCCA | |
| | | GAGGTGACATGTGTCGTGGTTGACGTGTCCCACGAAGATCCCGAA | |
| | | GTTAAGTTCAATTGGTATGTTGATGGTGTAGAGGTACACAATGCT | |
| | | AAGACTAAACCTCGCGAGGAGCAGTACAATTCGACCTATCGTGTC | |
| | | GTGAGCGTTCTGACCGTCCTTCACCAAGATTGGCTTAACGGCAAA | |
| | | GAATATAAGTGCAAGGTAAGCAATAAAGCACTTCCGGCCCCAAT | |
| | | CGAGAAAACCATTTCCAAGGCCAAAGGTCAACCAAGAGAACCCC | |
| | | AGGTGTATACTCTTCCGCCTTCTCGTGAGGAAATGACTAAAAATC | |
| | | AAGTATCCCTTACGTGTCTGGTTAAAGGTTTTTATCCTAGCGATAT | |
| | | TGCTGTTGAATGGGAATCGAACGGTCAGCCGGAGAATAATTATA | |
| | | AAACAACGCCACCCGTCCTGGATAGCGACGGCTCATTTTTTCTGT | |
| | | ATAGCAAACTGACTGTAGATAAATCACGGTGGCAGCAGGGCAAT | |
| | | GTATTCAGTTGCTCCGTTATGCATGAAGCGTTACATAATCACTAC | |
| | | ACGCAGAAATCTCTTAGTCTTTCACCCGGT | |
| Ab- | LC | GATATTGTGATGACTCAGTCTCCACTCTCCCTGCCCGTCACCCCTG | 79 |
| 007 | | GAGAGCCGGCCTCCATCTCCTGCAGGTCTAGTCAGAGCCTCCTGC | |
| | | ATAGTAATGGATACAACTATTTGGATTGGTACCTGCAGAAGCCAG | |
| | | GGCAGTCTCCACAGCTCCTGATCTATTTGGGTTCTAATCGGGCCTC | |
| | | CGGGGTCCCTGACAGGTTCAGTGGCAGTGGATCAGGCACAGATTT | |
| | | TACACTGAAAATCAGCAGAGTGGAGGCTGAGGATGTTGGGGTTT | |
| | | ATTACTGCATGCAAGCTCTACAAACTCCGTGGACGTTCGGCCAAG | |
| | | GGACCAAGCTGGAAATCAAACGTACGGTAGCTGCCCCTTCAGTTT | |
| | | TTATCTTTCCGCCGTCTGACGAGCAGTTAAAATCCGGGACCGCTT | |
| | | CTGTAGTTTGCCTGCTGAATAATTTTTATCCGCGTGAGGCTAAAGT | |
| | | ACAATGGAAAGTCGACAATGCTTTGCAGTCGGGAAATTCACAGG | |
| | | AAAGTGTTACGGAGCAGGATTCTAAAGATTCCACATATTCACTCA | |
| | | GCTCCACCCTTACACTGAGCAAAGCCGACTATGAAAAACATAAA | |
| | | GTTTACGCATGTGAGGTGACGCACCAAGGATTATCCAGTCCGGTC | |
| | | ACAAAATCGTTTAACCGCGGTGAGTGT | |
| Ab- | VH | GAAGTGCAGCTGGTGGAGTCTGGGGGAGGCTTGGTACAGCCTGG | 85 |
| 008 | | CAGGTCCCTGAGACTCTCCTGTACAACCTCTGGATTCACCTTTGCT | |
| | | GATTATGCCATGCACTGGGTCCGGCAAACTCCAGGGCAGGGCCTG | |
| | | GAGTGGGTCTCAGGTATTAGTTGGAATGGTGGTACCATGGGCTAT | |
| | | GCGGACTCTGTGAAGGGCCGATTCACCATCTCCAGAGACATCGCC | |
| | | AAGAACTCCCTGTATCTGCAAATGAACAGTCTGAGAGCTGAGGA | |
| | | CACGGCCTTATATTATTGTGCAAAGGATATCGCATATAGCAGAGG | |
| | | ATACTTCCAGCACTGGGGCCAGGGCACCCTGGTCACCGTCTCCTC | |
| | | A | |
| Ab- | VL | GACATCCAGATGACCCAGTCTCCATCCTCCCTGTCTGCATCTGTA | 86 |
| 008 | | GGAGACAGAGTCACCATCACTTGCCGGGCAAGTCAGGGCATTAG | |
| | | AAATGATTTAGGCTGGTATCAGCAGAAACCAGGGAAAGCCCCTA | |
| | | AGCTCCTGATCTATGCTGCATCCACTTTGCAATCAGGGGTCCCAT | |
| | | CTCGGTTCAGTGGCAGTGGATCTGGGACAGATTTCACTCTCACCA | |
| | | TCAGCAGCCTGCAGCCTGAAGATGTTGCAACTTATTACTGTCAAA | |
| | | AGTATAACAGTGCCCCATTCACTTTCGGCCCTGGGACCAAAGTGG | |
| | | ATATCAAA | |
| Ab- | HC | GAAGTGCAGCTGGTGGAGTCTGGGGGAGGCTTGGTACAGCCTGG | 89 |
| 008 | | CAGGTCCCTGAGACTCTCCTGTACAACCTCTGGATTCACCTTTGCT | |
| | | GATTATGCCATGCACTGGGTCCGGCAAACTCCAGGGCAGGGCCTG | |
| | | GAGTGGGTCTCAGGTATTAGTTGGAATGGTGGTACCATGGGCTAT | |
| | | GCGGACTCTGTGAAGGGCCGATTCACCATCTCCAGAGACATCGCC | |
| | | AAGAACTCCCTGTATCTGCAAATGAACAGTCTGAGAGCTGAGGA | |
| | | CACGGCCTTATATTATTGTGCAAAGGATATCGCATATAGCAGAGG | |
| | | ATACTTCCAGCACTGGGGCCAGGGCACCCTGGTCACCGTCTCCTC | |
| | | AGCTAGCACTAAAGGGCCTTCTGTATTTCCCTTGGCCCCGTCCAG | |
| | | CAAATCGACCTCGGGAGGGACAGCCGCCCTGGGTTGCCTTGTGAA | |
| | | AGATTATTTCCCTGAGCCAGTTACCGTAAGTTGGAACAGTGGGGC | |
| | | GCTGACAAGTGGTGTGCACACGTTTCCTGCCGTCCTGCAATCATC | |
| | | GGGCTTGTATAGCCTCAGCTCTGTGGTCACTGTCCCAAGTTCATC | |
| | | GCTGGGCACTCAGACGTATATTTGCAATGTGAACCACAAACCTTC | |
| | | AAATACAAAAGTGGATAAACGCGTAGAACCGAAATCGTGTGATA | |
| | | AAACTCACACATGCCCGCCATGCCCGGCACCTGAACTGCTTGGTG | |
| | | GTCCCAGCGTGTTCCTGTTCCCGCCGAAGCCTAAAGATACTCTAA | |
| | | TGATCAGCCGTACGCCAGAGGTGACATGTGTCGTGGTTGACGTGT | |
| | | CCCACGAAGATCCCGAAGTTAAGTTCAATTGGTATGTTGATGGTG | |
| | | TAGAGGTACACAATGCTAAGACTAAACCTCGCGAGGAGCAGTAC | |
| | | AATTCGACCTATCGTGTCGTGAGCGTTCTGACCGTCCTTCACCAA | |
| | | GATTGGCTTAACGGCAAAGAATATAAGTGCAAGGTAAGCAATAA | |
| | | AGCACTTCCGGCCCCAATCGAGAAAACCATTICCAAGGCCAAAG | |
| | | GTCAACCAAGAGAACCCCAGGTGTATACTCTTCCGCCTTCTCGTG | |
| | | AGGAAATGACTAAAAATCAAGTATCCCTTACGTGTCTGGTTAAAG | |
| | | GTTTTTATCCTAGCGATATTGCTGTTGAATGGGAATCGAACGGTC | |
| | | AGCCGGAGAATAATTATAAAACAACGCCACCCGTCCTGGATAGC | |
| | | GACGGCTCATTTTTTCTGTATAGCAAACTGACTGTAGATAAATCA | |
| | | CGGTGGCAGCAGGGCAATGTATTCAGTTGCTCCGTTATGCATGAA | |
| | | GCGTTACATAATCACTACACGCAGAAATCTCTTAGTCTTTCACCC | |
| | | GGT | |
| Ab- | LC | GACATCCAGATGACCCAGTCTCCATCCTCCCTGTCTGCATCTGTA | 90 |
| 008 | | GGAGACAGAGTCACCATCACTTGCCGGGCAAGTCAGGGCATTAG | |
| | | AAATGATTTAGGCTGGTATCAGCAGAAACCAGGGAAAGCCCCTA | |
| | | AGCTCCTGATCTATGCTGCATCCACTTTGCAATCAGGGGTCCCAT | |
| | | CTCGGTTCAGTGGCAGTGGATCTGGGACAGATTTCACTCTCACCA | |
| | | TCAGCAGCCTGCAGCCTGAAGATGTTGCAACTTATTACTGTCAAA | |
| | | AGTATAACAGTGCCCCATTCACTTTCGGCCCTGGGACCAAAGTGG | |
| | | ATATCAAACGTACGGTAGCTGCCCCTTCAGTTTTTATCTTTCCGCC | |
| | | GTCTGACGAGCAGTTAAAATCCGGGACCGCTTCTGTAGTTTGCCT | |
| | | GCTGAATAATTTTTATCCGCGTGAGGCTAAAGTACAATGGAAAGT | |
| | | CGACAATGCTTTGCAGTCGGGAAATTCACAGGAAAGTGTTACGG | |
| | | AGCAGGATTCTAAAGATTCCACATATTCACTCAGCTCCACCCTTA | |
| | | CACTGAGCAAAGCCGACTATGAAAAACATAAAGTTTACGCATGT | |
| | | GAGGTGACGCACCAAGGATTATCCAGTCCGGTCACAAAATCGTTT | |
| | | AACCGCGGTGAGTGT | |
| Ab- | VH | GAAGTGCAGCTGGTGGAGTCTGGGGGAGGCTTGGTACAGCCTGG | 96 |
| 009 | | CAGGTCCCTGAGACTCTCCTGTACAACCTCTGGATTCACCTTTGCT | |
| | | GATTATGCCATGCACTGGGTCCGGCAAACTCCAGGGAAGGGCCT | |
| | | GGAGTGGGTCTCAGGTATTAGTTGGAATGGTGTTACCATTGACTA | |
| | | TGCGGACTCTGTGAAGGGCCGATTCACCATCTCCAGAGACATCGC | |
| | | CAAGAACTCCCTGTATCTGCAAATGAACAGTCTGAGAGCTGAGG | |
| | | ACACGGCCTTATATTATTGTGCAAAGGATATAGCATATAGCAGAG | |
| | | GATACTTCCAGCACTGGGGCCAGGGCACCCTGGTCACTGTCTCCT | |
| | | CA | |
| Ab- | VL | GACATCGTGATGACCCAGTCTCCAGACTCCCTGGCTGTGTCTCTG | 97 |
| 009 | | GGCGAGAGGGCCACCGTCAACTGCAAGTCCAGCCAGAATGTTTT | |
| | | ATACAGTTCCAACAATGAGAATCACTTAGCTTGGTACCAGCAGAA | |
| | | ACCAGGACAGCCTCCTAAGCTGCTCATTTACTGGGCATCTACCCG | |
| | | GGAATCCGGGGTCCCTGACCGATTCAGTGGCAGCGGGTCTGGGA | |
| | | CAGATTTCACTCTCACCATCAGCAGCCTGCAGGCTGAAGATGTGG | |
| | | CAATTTATTACTGTCAACAATATTATAGTACTCCGTACACTTTTGG | |
| | | CCAGGGGACCAAAGTGGATATCAAA | |
| Ab- | HC | GAAGTGCAGCTGGTGGAGTCTGGGGGAGGCTTGGTACAGCCTGG | 100 |
| 009 | | CAGGTCCCTGAGACTCTCCTGTACAACCTCTGGATTCACCTTTGCT | |
| | | GATTATGCCATGCACTGGGTCCGGCAAACTCCAGGGAAGGGCCT | |
| | | GGAGTGGGTCTCAGGTATTAGTTGGAATGGTGTTACCATTGACTA | |
| | | TGCGGACTCTGTGAAGGGCCGATTCACCATCTCCAGAGACATCGC | |
| | | CAAGAACTCCCTGTATCTGCAAATGAACAGTCTGAGAGCTGAGG | |
| | | ACACGGCCTTATATTATTGTGCAAAGGATATAGCATATAGCAGAG | |
| | | GATACTTCCAGCACTGGGGCCAGGGCACCCTGGTCACTGTCTCCT | |
| | | CAGCTAGCACTAAAGGGCCTTCTGTATTTCCCTTGGCCCCGTCCA | |
| | | GCAAATCGACCTCGGGAGGGACAGCCGCCCTGGGTTGCCTTGTGA | |
| | | AAGATTATTTCCCTGAGCCAGTTACCGTAAGTTGGAACAGTGGGG | |
| | | CGCTGACAAGTGGTGTGCACACGTTTCCTGCCGTCCTGCAATCAT | |
| | | CGGGCTTGTATAGCCTCAGCTCTGTGGTCACTGTCCCAAGTTCATC | |
| | | GCTGGGCACTCAGACGTATATTTGCAATGTGAACCACAAACCTTC | |
| | | AAATACAAAAGTGGATAAACGCGTAGAACCGAAATCGTGTGATA | |
| | | AAACTCACACATGCCCGCCATGCCCGGCACCTGAACTGCTTGGTG | |
| | | GTCCCAGCGTGTTCCTGTTCCCGCCGAAGCCTAAAGATACTCTAA | |
| | | TGATCAGCCGTACGCCAGAGGTGACATGTGTCGTGGTTGACGTGT | |
| | | CCCACGAAGATCCCGAAGTTAAGTTCAATTGGTATGTTGATGGTG | |
| | | TAGAGGTACACAATGCTAAGACTAAACCTCGCGAGGAGCAGTAC | |
| | | AATTCGACCTATCGTGTCGTGAGCGTTCTGACCGTCCTTCACCAA | |
| | | GATTGGCTTAACGGCAAAGAATATAAGTGCAAGGTAAGCAATAA | |
| | | AGCACTTCCGGCCCCAATCGAGAAAACCATTTCCAAGGCCAAAG | |
| | | GTCAACCAAGAGAACCCCAGGTGTATACTCTTCCGCCTTCTCGTG | |
| | | AGGAAATGACTAAAAATCAAGTATCCCTTACGTGTCTGGTTAAAG | |
| | | GTTTTTATCCTAGCGATATTGCTGTTGAATGGGAATCGAACGGTC | |
| | | AGCCGGAGAATAATTATAAAACAACGCCACCCGTCCTGGATAGC | |
| | | GACGGCTCATTTTTTCTGTATAGCAAACTGACTGTAGATAAATCA | |
| | | CGGTGGCAGCAGGGCAATGTATTCAGTTGCTCCGTTATGCATGAA | |
| | | GCGTTACATAATCACTACACGCAGAAATCTCTTAGTCTTTCACCC | |
| | | GGT | |
| Ab- | LC | GACATCGTGATGACCCAGTCTCCAGACTCCCTGGCTGTGTCTCTG | 101 |
| 009 | | GGCGAGAGGGCCACCGTCAACTGCAAGTCCAGCCAGAATGTTTT | |
| | | ATACAGTTCCAACAATGAGAATCACTTAGCTTGGTACCAGCAGAA | |
| | | ACCAGGACAGCCTCCTAAGCTGCTCATTTACTGGGCATCTACCCG | |
| | | GGAATCCGGGGTCCCTGACCGATTCAGTGGCAGCGGGTCTGGGA | |
| | | CAGATTTCACTCTCACCATCAGCAGCCTGCAGGCTGAAGATGTGG | |
| | | CAATTTATTACTGTCAACAATATTATAGTACTCCGTACACTTTTGG | |
| | | CCAGGGGACCAAAGTGGATATCAAACGTACGGTAGCTGCCCCTTC | |
| | | AGTTTTTATCTTTCCGCCGTCTGACGAGCAGTTAAAATCCGGGAC | |
| | | CGCTTCTGTAGTTTGCCTGCTGAATAATTTTTATCCGCGTGAGGCT | |
| | | AAAGTACAATGGAAAGTCGACAATGCTTTGCAGTCGGGAAATTC | |
| | | ACAGGAAAGTGTTACGGAGCAGGATTCTAAAGATTCCACATATTC | |
| | | ACTCAGCTCCACCCTTACACTGAGCAAAGCCGACTATGAAAAACA | |
| | | TAAAGTTTACGCATGTGAGGTGACGCACCAAGGATTATCCAGTCC | |
| | | GGTCACAAAATCGTTTAACCGCGGTGAGTGT | |
| Ab- | VH | GAAGTGCAGCTGGTGGAGTCTGGGGGAGGCTTGGTACAGCCTGG | 106 |
| 010 | | GGGGTCCCTGAGACTCTCCTGTACATCCTCTGGATTCACCTTTGCT | |
| | | GATTATGCCATGCACTGGGTCCGGCAAACTCCAGGGAAGGGCCT | |
| | | GGAGTGGGTCTCAGGTATTAGTTGGAATGGTGGTACCTTTGACTA | |
| | | TGCGGACTCTGTGAAGGGCCGATTCACCATCTCCAGAGACATCGC | |
| | | CAAGAACTCCCTGTATCTGCAAATGAACAGTCTGAGAGTTGAGGA | |
| | | CACGGCCTTATATTATTGTGCAAAGGATATAGCATATAGCAGAGG | |
| | | ATACTTCCAGCACTGGGGCCAGGGCACCCTGGTCACCGTCTCCTC | |
| | | A | |
| Ab- | VL | GAAATTGTGTTGACGCAGTCTCCAGGCACCCTGTCTTTGTCTCCA | 107 |
| 010 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCACCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCTCTCACTTTCGGCGGAGGGACCAAGGT | |
| | | GGAGATCAAA | |
| Ab- | HC | GAAGTGCAGCTGGTGGAGTCTGGGGGAGGCTTGGTACAGCCTGG | 110 |
| 010 | | GGGGTCCCTGAGACTCTCCTGTACATCCTCTGGATTCACCTTTGCT | |
| | | GATTATGCCATGCACTGGGTCCGGCAAACTCCAGGGAAGGGCCT | |
| | | GGAGTGGGTCTCAGGTATTAGTTGGAATGGTGGTACCTTTGACTA | |
| | | TGCGGACTCTGTGAAGGGCCGATTCACCATCTCCAGAGACATCGC | |
| | | CAAGAACTCCCTGTATCTGCAAATGAACAGTCTGAGAGTTGAGGA | |
| | | CACGGCCTTATATTATTGTGCAAAGGATATAGCATATAGCAGAGG | |
| | | ATACTTCCAGCACTGGGGCCAGGGCACCCTGGTCACCGTCTCCTC | |
| | | AGCTAGCACTAAAGGGCCTTCTGTATTTCCCTTGGCCCCGTCCAG | |
| | | CAAATCGACCTCGGGAGGGACAGCCGCCCTGGGTTGCCTTGTGAA | |
| | | AGATTATTTCCCTGAGCCAGTTACCGTAAGTTGGAACAGTGGGGC | |
| | | GCTGACAAGTGGTGTGCACACGTTTCCTGCCGTCCTGCAATCATC | |
| | | GGGCTTGTATAGCCTCAGCTCTGTGGTCACTGTCCCAAGTTCATC | |
| | | GCTGGGCACTCAGACGTATATTTGCAATGTGAACCACAAACCTTC | |
| | | AAATACAAAAGTGGATAAACGCGTAGAACCGAAATCGTGTGATA | |
| | | AAACTCACACATGCCCGCCATGCCCGGCACCTGAACTGCTTGGTG | |
| | | GTCCCAGCGTGTTCCTGTTCCCGCCGAAGCCTAAAGATACTCTAA | |
| | | TGATCAGCCGTACGCCAGAGGTGACATGTGTCGTGGTTGACGTGT | 111 |
| | | CCCACGAAGATCCCGAAGTTAAGTTCAATTGGTATGTTGATGGTG | |
| | | TAGAGGTACACAATGCTAAGACTAAACCTCGCGAGGAGCAGTAC | |
| | | AATTCGACCTATCGTGTCGTGAGCGTTCTGACCGTCCTTCACCAA | |
| | | GATTGGCTTAACGGCAAAGAATATAAGTGCAAGGTAAGCAATAA | |
| | | AGCACTTCCGGCCCCAATCGAGAAAACCATTTCCAAGGCCAAAG | |
| | | GTCAACCAAGAGAACCCCAGGTGTATACTCTTCCGCCTTCTCGTG | |
| | | AGGAAATGACTAAAAATCAAGTATCCCTTACGTGTCTGGTTAAAG | |
| | | GTTTTTATCCTAGCGATATTGCTGTTGAATGGGAATCGAACGGTC | |
| | | AGCCGGAGAATAATTATAAAACAACGCCACCCGTCCTGGATAGC | |
| | | GACGGCTCATTTTTTCTGTATAGCAAACTGACTGTAGATAAATCA | |
| | | CGGTGGCAGCAGGGCAATGTATTCAGTTGCTCCGTTATGCATGAA | |
| | | GCGTTACATAATCACTACACGCAGAAATCTCTTAGTCTTTCACCC | |
| | | GGT | |
| Ab- | LC | GAAATTGTGTTGACGCAGTCTCCAGGCACCCTGTCTTTGTCTCCA | |
| 010 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCACCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCTCTCACTTTCGGCGGAGGGACCAAGGT | |
| | | GGAGATCAAACGTACGGTAGCTGCCCCTTCAGTTTTTATCTTTCCG | |
| | | CCGTCTGACGAGCAGTTAAAATCCGGGACCGCTTCTGTAGTTTGC | |
| | | CTGCTGAATAATTTTTATCCGCGTGAGGCTAAAGTACAATGGAAA | |
| | | GTCGACAATGCTTTGCAGTCGGGAAATTCACAGGAAAGTGTTACG | |
| | | GAGCAGGATTCTAAAGATTCCACATATTCACTCAGCTCCACCCTT | |
| | | ACACTGAGCAAAGCCGACTATGAAAAACATAAAGTTTACGCATG | |
| | | TGAGGTGACGCACCAAGGATTATCCAGTCCGGTCACAAAATCGTT | |
| | | TAACCGCGGTGAGTGT | |
| Ab- | VH | GAAGTGCAGCTGGTGGAGTCTGGGGGAGGCTTGGTACAGCCTGG | 116 |
| 012 | | CAGGTCCCTGAGACTCTCCTGTACAACCTCTGGATTCACCTTTGCT | |
| | | GATTATGCCATGCACTGGGTCCGGCAAACTCCAGGGCAGGGCCTG | |
| | | GAGTGGGTCTCAGGTATTAGTTGGAATGGTGGTACCATGGGCTAT | |
| | | GCGGACTCTGTGAAGGGCCGATTCACCATCTCCAGAGACATCGCC | |
| | | AAGAACTCCCTGTATCTGCAAATGAACAGTCTGAGAGCTGAGGA | |
| | | CACGGCCTTATATTATTGTGCAAAGGATATCGCATATAGCAGAGG | |
| | | ATACTTCCAGCACTGGGGCCAGGGCACCCTGGTCACCGTCTCCTC | |
| | | A | |
| Ab- | VL | GAAATTGTGTTGACGCAGTCTCCAGCCACCCTGTCTGTGTCTCCA | 117 |
| 012 | | GGGGAAAGAGCCACCCTCTCCTGCAGGGCCAGTCAGAGTGTTAG | |
| | | CAGCAACTTAGCCTGGTACCAGCAGAAACCTGGCCAGGCTCCCA | |
| | | GGCTCCTCATCTATGGTGCATCCAGCAGGGCCACTGGCATCCCAG | |
| | | ACAGGTTCAGTGGCAGTGGGTCTGGGACAGACTTCACTCTCACCA | |
| | | TCAGCAGACTGGAGCCTGAAGATTTTGCAGTGTATTACTGTCAGC | |
| | | AGTATGGTAGCTCACAGATCACCTTCGGCCAAGGGACACGACTG | |
| | | GAGATTAAA | |
| Ab- | HC | GAAGTGCAGCTGGTGGAGTCTGGGGGAGGCTTGGTACAGCCTGG | 120 |
| 012 | | CAGGTCCCTGAGACTCTCCTGTACAACCTCTGGATTCACCTTTGCT | |
| | | GATTATGCCATGCACTGGGTCCGGCAAACTCCAGGGCAGGGCCTG | |
| | | GAGTGGGTCTCAGGTATTAGTTGGAATGGTGGTACCATGGGCTAT | |
| | | GCGGACTCTGTGAAGGGCCGATTCACCATCTCCAGAGACATCGCC | |
| | | AAGAACTCCCTGTATCTGCAAATGAACAGTCTGAGAGCTGAGGA | |
| | | CACGGCCTTATATTATTGTGCAAAGGATATCGCATATAGCAGAGG | |
| | | ATACTTCCAGCACTGGGGCCAGGGCACCCTGGTCACCGTCTCCTC | |
| | | AGCTAGCACTAAAGGGCCTTCTGTATTTCCCTTGGCCCCGTCCAG | |
| | | CAAATCGACCTCGGGAGGGACAGCCGCCCTGGGTTGCCTTGTGAA | |
| | | AGATTATTTCCCTGAGCCAGTTACCGTAAGTTGGAACAGTGGGGC | |
| | | GCTGACAAGTGGTGTGCACACGTTTCCTGCCGTCCTGCAATCATC | |
| | | GGGCTTGTATAGCCTCAGCTCTGTGGTCACTGTCCCAAGTTCATC | |
| | | GCTGGGCACTCAGACGTATATTTGCAATGTGAACCACAAACCTTC | |
| | | AAATACAAAAGTGGATAAACGCGTAGAACCGAAATCGTGTGATA | |
| | | AAACTCACACATGCCCGCCATGCCCGGCACCTGAACTGCTTGGTG | |
| | | GTCCCAGCGTGTTCCTGTTCCCGCCGAAGCCTAAAGATACTCTAA | |
| | | TGATCAGCCGTACGCCAGAGGTGACATGTGTCGTGGTTGACGTGT | |
| | | CCCACGAAGATCCCGAAGTTAAGTTCAATTGGTATGTTGATGGTG | |
| | | TAGAGGTACACAATGCTAAGACTAAACCTCGCGAGGAGCAGTAC | |
| | | AATTCGACCTATCGTGTCGTGAGCGTTCTGACCGTCCTTCACCAA | |
| | | GATTGGCTTAACGGCAAAGAATATAAGTGCAAGGTAAGCAATAA | |
| | | AGCACTTCCGGCCCCAATCGAGAAAACCATTTCCAAGGCCAAAG | |
| | | GTCAACCAAGAGAACCCCAGGTGTATACTCTTCCGCCTTCTCGTG | |
| | | AGGAAATGACTAAAAATCAAGTATCCCTTACGTGTCTGGTTAAAG | |
| | | GTTTTTATCCTAGCGATATTGCTGTTGAATGGGAATCGAACGGTC | |
| | | AGCCGGAGAATAATTATAAAACAACGCCACCCGTCCTGGATAGC | |
| | | GACGGCTCATTTTTTCTGTATAGCAAACTGACTGTAGATAAATCA | |
| | | CGGTGGCAGCAGGGCAATGTATTCAGTTGCTCCGTTATGCATGAA | |
| | | GCGTTACATAATCACTACACGCAGAAATCTCTTAGTCTTTCACCC | |
| | | GGT | |
| Ab- | LC | GAAATTGTGTTGACGCAGTCTCCAGCCACCCTGTCTGTGTCTCCA | 121 |
| 012 | | GGGGAAAGAGCCACCCTCTCCTGCAGGGCCAGTCAGAGTGTTAG | |
| | | CAGCAACTTAGCCTGGTACCAGCAGAAACCTGGCCAGGCTCCCA | |
| | | GGCTCCTCATCTATGGTGCATCCAGCAGGGCCACTGGCATCCCAG | |
| | | ACAGGTTCAGTGGCAGTGGGTCTGGGACAGACTTCACTCTCACCA | |
| | | TCAGCAGACTGGAGCCTGAAGATTTTGCAGTGTATTACTGTCAGC | |
| | | AGTATGGTAGCTCACAGATCACCTTCGGCCAAGGGACACGACTG | |
| | | GAGATTAAACGTACGGTAGCTGCCCCTTCAGTTTTTATCTTTCCGC | |
| | | CGTCTGACGAGCAGTTAAAATCCGGGACCGCTTCTGTAGTTTGCC | |
| | | TGCTGAATAATTTTTATCCGCGTGAGGCTAAAGTACAATGGAAAG | |
| | | TCGACAATGCTTTGCAGTCGGGAAATTCACAGGAAAGTGTTACGG | |
| | | AGCAGGATTCTAAAGATTCCACATATTCACTCAGCTCCACCCTTA | |
| | | CACTGAGCAAAGCCGACTATGAAAAACATAAAGTTTACGCATGT | |
| | | GAGGTGACGCACCAAGGATTATCCAGTCCGGTCACAAAATCGTTT | |
| | | AACCGCGGTGAGTGT | |
| Ab- | VH | GAAGTGCAGCTGGTGGAGTCTGGGGGAGGCTTGGTACAGCCTGG | 124 |
| 014 | | CAGGTCCCTGAGACTCTCCTGTACAACCTCTGGATTCACCTTTGCT | |
| | | GATTATGCCATGCACTGGGTCCGGCAAACTCCAGGGAAGGGCCT | |
| | | GGAGTGGGTCTCAGGTATTAGTTGGAATGGTGTTACCATTGACTA | |
| | | TGCGGACTCTGTGAAGGGCCGATTCACCATCTCCAGAGACATCGC | |
| | | CAAGAACTCCCTGTATCTGCAAATGAACAGTCTGAGAGCTGAGG | |
| | | ACACGGCCTTATATTATTGTGCAAAGGATATAGCATATAGCAGAG | |
| | | GATACTTCCAGCACTGGGGCCAGGGCACCCTGGTCACCGTCTCTT | |
| | | CA | |
| Ab- | VL | GAAATTGTGTTGACGCAGTCTCCAGGCACCCTGTCTTTGTCTCCA | 125 |
| 014 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCGCTCACTTTCGGCGGAGGGACCAAAGT | |
| | | GGATATCAAA | |
| Ab- | HC | GAAGTGCAGCTGGTGGAGTCTGGGGGAGGCTTGGTACAGCCTGG | 128 |
| 014 | | CAGGTCCCTGAGACTCTCCTGTACAACCTCTGGATTCACCTTTGCT | |
| | | GATTATGCCATGCACTGGGTCCGGCAAACTCCAGGGAAGGGCCT | |
| | | GGAGTGGGTCTCAGGTATTAGTTGGAATGGTGTTACCATTGACTA | |
| | | TGCGGACTCTGTGAAGGGCCGATTCACCATCTCCAGAGACATCGC | |
| | | CAAGAACTCCCTGTATCTGCAAATGAACAGTCTGAGAGCTGAGG | |
| | | ACACGGCCTTATATTATTGTGCAAAGGATATAGCATATAGCAGAG | |
| | | GATACTTCCAGCACTGGGGCCAGGGCACCCTGGTCACCGTCTCTT | |
| | | CAGCTAGCACTAAAGGGCCTTCTGTATTTCCCTTGGCCCCGTCCA | |
| | | GCAAATCGACCTCGGGAGGGACAGCCGCCCTGGGTTGCCTTGTGA | |
| | | AAGATTATTTCCCTGAGCCAGTTACCGTAAGTTGGAACAGTGGGG | |
| | | CGCTGACAAGTGGTGTGCACACGTTTCCTGCCGTCCTGCAATCAT | |
| | | CGGGCTTGTATAGCCTCAGCTCTGTGGTCACTGTCCCAAGTTCATC | |
| | | GCTGGGCACTCAGACGTATATTTGCAATGTGAACCACAAACCTTC | |
| | | AAATACAAAAGTGGATAAACGCGTAGAACCGAAATCGTGTGATA | |
| | | AAACTCACACATGCCCGCCATGCCCGGCACCTGAACTGCTTGGTG | |
| | | GTCCCAGCGTGTTCCTGTTCCCGCCGAAGCCTAAAGATACTCTAA | |
| | | TGATCAGCCGTACGCCAGAGGTGACATGTGTCGTGGTTGACGTGT | |
| | | CCCACGAAGATCCCGAAGTTAAGTTCAATTGGTATGTTGATGGTG | |
| | | TAGAGGTACACAATGCTAAGACTAAACCTCGCGAGGAGCAGTAC | |
| | | AATTCGACCTATCGTGTCGTGAGCGTTCTGACCGTCCTTCACCAA | |
| | | GATTGGCTTAACGGCAAAGAATATAAGTGCAAGGTAAGCAATAA | |
| | | AGCACTTCCGGCCCCAATCGAGAAAACCATTTCCAAGGCCAAAG | |
| | | GTCAACCAAGAGAACCCCAGGTGTATACTCTTCCGCCTTCTCGTG | |
| | | AGGAAATGACTAAAAATCAAGTATCCCTTACGTGTCTGGTTAAAG | |
| | | GTTTTTATCCTAGCGATATTGCTGTTGAATGGGAATCGAACGGTC | |
| | | AGCCGGAGAATAATTATAAAACAACGCCACCCGTCCTGGATAGC | |
| | | GACGGCTCATTTTTTCTGTATAGCAAACTGACTGTAGATAAATCA | |
| | | CGGTGGCAGCAGGGCAATGTATTCAGTTGCTCCGTTATGCATGAA | |
| | | GCGTTACATAATCACTACACGCAGAAATCTCTTAGTCTTTCACCC | |
| | | GGT | |
| Ab- | LC | GAAATTGTGTTGACGCAGTCTCCAGGCACCCTGTCTTTGTCTCCA | 129 |
| 014 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCGCTCACTTTCGGCGGAGGGACCAAAGT | |
| | | GGATATCAAACGTACGGTAGCTGCCCCTTCAGTTTTTATCTTTCCG | |
| | | CCGTCTGACGAGCAGTTAAAATCCGGGACCGCTTCTGTAGTTTGC | |
| | | CTGCTGAATAATTTTTATCCGCGTGAGGCTAAAGTACAATGGAAA | |
| | | GTCGACAATGCTTTGCAGTCGGGAAATTCACAGGAAAGTGTTACG | |
| | | GAGCAGGATTCTAAAGATTCCACATATTCACTCAGCTCCACCCTT | |
| | | ACACTGAGCAAAGCCGACTATGAAAAACATAAAGTTTACGCATG | |
| | | TGAGGTGACGCACCAAGGATTATCCAGTCCGGTCACAAAATCGTT | |
| | | TAACCGCGGTGAGTGT | |
| Ab- | VH | GAAGTGCAGCTGGTGGAGTCTGGGGGAGGCTTGGTACAGCCTGG | 133 |
| 016 | | CAGGTCCCTGAGACTCTCCTGTACAACCTCTGGATTCACCTTTGCT | |
| | | GATTATGCCATGCACTGGGTCCGGCAAACTCCAGGGAAGGGCCT | |
| | | GGAGTGGGTCTCAGGTATTAGTTGGAATGGTGGTACCATAGGCTA | |
| | | TGCGGACTCTGTGAAGGGCCGATTCACCATCTCCAGAGACATCGC | |
| | | CAAGAACTCCCTGTATCTGCAAATGAACAGTCTGAGAGCTGAGG | |
| | | ACACGGCCTTATATTATTGTGCAAAGGATATAGCATATAGCAGAG | |
| | | GATACTTCCAGCACTGGGGCCAGGGCACCCTGGTCACCGTCTCCT | |
| | | CA | |
| Ab- | VL | GAAATTGTGTTGACGCAGTCTCCAGGCACCCTGTCTTTGTCTCCA | 134 |
| 016 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCTCACACTTTTGGCCAGGGGACCAAGGT | |
| | | GGAAATCAAA | |
| Ab- | HC | GAAGTGCAGCTGGTGGAGTCTGGGGGAGGCTTGGTACAGCCTGG | 137 |
| 016 | | CAGGTCCCTGAGACTCTCCTGTACAACCTCTGGATTCACCTTTGCT | |
| | | GATTATGCCATGCACTGGGTCCGGCAAACTCCAGGGAAGGGCCT | |
| | | GGAGTGGGTCTCAGGTATTAGTTGGAATGGTGGTACCATAGGCTA | |
| | | TGCGGACTCTGTGAAGGGCCGATTCACCATCTCCAGAGACATCGC | |
| | | CAAGAACTCCCTGTATCTGCAAATGAACAGTCTGAGAGCTGAGG | |
| | | ACACGGCCTTATATTATTGTGCAAAGGATATAGCATATAGCAGAG | |
| | | GATACTTCCAGCACTGGGGCCAGGGCACCCTGGTCACCGTCTCCT | |
| | | CAGCTAGCACTAAAGGGCCTTCTGTATTTCCCTTGGCCCCGTCCA | |
| | | GCAAATCGACCTCGGGAGGGACAGCCGCCCTGGGTTGCCTTGTGA | |
| | | AAGATTATTTCCCTGAGCCAGTTACCGTAAGTTGGAACAGTGGGG | |
| | | CGCTGACAAGTGGTGTGCACACGTTTCCTGCCGTCCTGCAATCAT | |
| | | CGGGCTTGTATAGCCTCAGCTCTGTGGTCACTGTCCCAAGTTCATC | |
| | | GCTGGGCACTCAGACGTATATTTGCAATGTGAACCACAAACCTTC | |
| | | AAATACAAAAGTGGATAAACGCGTAGAACCGAAATCGTGTGATA | |
| | | AAACTCACACATGCCCGCCATGCCCGGCACCTGAACTGCTTGGTG | |
| | | GTCCCAGCGTGTTCCTGTTCCCGCCGAAGCCTAAAGATACTCTAA | |
| | | TGATCAGCCGTACGCCAGAGGTGACATGTGTCGTGGTTGACGTGT | |
| | | CCCACGAAGATCCCGAAGTTAAGTTCAATTGGTATGTTGATGGTG | |
| | | TAGAGGTACACAATGCTAAGACTAAACCTCGCGAGGAGCAGTAC | |
| | | AATTCGACCTATCGTGTCGTGAGCGTTCTGACCGTCCTTCACCAA | |
| | | GATTGGCTTAACGGCAAAGAATATAAGTGCAAGGTAAGCAATAA | |
| | | AGCACTTCCGGCCCCAATCGAGAAAACCATTTCCAAGGCCAAAG | |
| | | GTCAACCAAGAGAACCCCAGGTGTATACTCTTCCGCCTTCTCGTG | |
| | | AGGAAATGACTAAAAATCAAGTATCCCTTACGTGTCTGGTTAAAG | |
| | | GTTTTTATCCTAGCGATATTGCTGTTGAATGGGAATCGAACGGTC | |
| | | AGCCGGAGAATAATTATAAAACAACGCCACCCGTCCTGGATAGC | |
| | | GACGGCTCATTTTTTCTGTATAGCAAACTGACTGTAGATAAATCA | |
| | | CGGTGGCAGCAGGGCAATGTATTCAGTTGCTCCGTTATGCATGAA | |
| | | GCGTTACATAATCACTACACGCAGAAATCTCTTAGTCTTTCACCC | |
| | | GGT | |
| Ab- | LC | GAAATTGTGTTGACGCAGTCTCCAGGCACCCTGTCTTTGTCTCCA | 138 |
| 016 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCTCACACTTTTGGCCAGGGGACCAAGGT | |
| | | GGAAATCAAACGTACGGTAGCTGCCCCTTCAGTTTTTATCTTTCCG | |
| | | CCGTCTGACGAGCAGTTAAAATCCGGGACCGCTTCTGTAGTTTGC | |
| | | CTGCTGAATAATTTTTATCCGCGTGAGGCTAAAGTACAATGGAAA | |
| | | GTCGACAATGCTTTGCAGTCGGGAAATTCACAGGAAAGTGTTACG | |
| | | GAGCAGGATTCTAAAGATTCCACATATTCACTCAGCTCCACCCTT | |
| | | ACACTGAGCAAAGCCGACTATGAAAAACATAAAGTTTACGCATG | |
| | | TGAGGTGACGCACCAAGGATTATCCAGTCCGGTCACAAAATCGTT | |
| | | TAACCGCGGTGAGTGT | |
| Ab- | VH | GAAGTGCAGCTGGTGGAGTCTGGGGGAGGCTTGGTACAGCCTGG | 141 |
| 017 | | CAGGTCCCTGAGACTCTCCTGTACAACCTCTGGATTCACCTTTGCT | |
| | | GATTATGCCATGCACTGGGTCCGGCAAACTCCAGGGCAGGGCCTG | |
| | | GAGTGGGTCTCAGGTATTAGTTGGAATGGTGGTACCATGGGCTAT | |
| | | GCGGACTCTGTGAAGGGCCGATTCACCATCTCCAGAGACATCGCC | |
| | | AAGAACTCCCTGTATCTGCAAATGAACAGTCTGAGAGCTGAGGA | |
| | | CACGGCCTTATATTATTGTGCAAAGGATATCGCATATAGCAGAGG | |
| | | ATACTTCCAGCACTGGGGCCAGGGCACCCTGGTCACTGTCTCCTC | |
| | | A | |
| Ab- | VL | GAAATTGTGTTGACGCAGTCTCCAGGCACCCTGTCTTTGTCTCCA | 142 |
| 017 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCGCTCACTTTCGGCGGAGGGACCAAAGT | |
| | | GGATATCAAA | |
| Ab- | HC | GAAGTGCAGCTGGTGGAGTCTGGGGGAGGCTTGGTACAGCCTGG | 145 |
| 017 | | CAGGTCCCTGAGACTCTCCTGTACAACCTCTGGATTCACCTTTGCT | |
| | | GATTATGCCATGCACTGGGTCCGGCAAACTCCAGGGCAGGGCCTG | |
| | | GAGTGGGTCTCAGGTATTAGTTGGAATGGTGGTACCATGGGCTAT | |
| | | GCGGACTCTGTGAAGGGCCGATTCACCATCTCCAGAGACATCGCC | |
| | | AAGAACTCCCTGTATCTGCAAATGAACAGTCTGAGAGCTGAGGA | |
| | | CACGGCCTTATATTATTGTGCAAAGGATATCGCATATAGCAGAGG | |
| | | ATACTTCCAGCACTGGGGCCAGGGCACCCTGGTCACTGTCTCCTC | |
| | | AGCTAGCACTAAAGGGCCTTCTGTATTTCCCTTGGCCCCGTCCAG | |
| | | CAAATCGACCTCGGGAGGGACAGCCGCCCTGGGTTGCCTTGTGAA | |
| | | AGATTATTTCCCTGAGCCAGTTACCGTAAGTTGGAACAGTGGGGC | |
| | | GCTGACAAGTGGTGTGCACACGTTTCCTGCCGTCCTGCAATCATC | |
| | | GGGCTTGTATAGCCTCAGCTCTGTGGTCACTGTCCCAAGTTCATC | |
| | | GCTGGGCACTCAGACGTATATTTGCAATGTGAACCACAAACCTTC | |
| | | AAATACAAAAGTGGATAAACGCGTAGAACCGAAATCGTGTGATA | |
| | | AAACTCACACATGCCCGCCATGCCCGGCACCTGAACTGCTTGGTG | |
| | | GTCCCAGCGTGTTCCTGTTCCCGCCGAAGCCTAAAGATACTCTAA | |
| | | TGATCAGCCGTACGCCAGAGGTGACATGTGTCGTGGTTGACGTGT | |
| | | CCCACGAAGATCCCGAAGTTAAGTTCAATTGGTATGTTGATGGTG | |
| | | TAGAGGTACACAATGCTAAGACTAAACCTCGCGAGGAGCAGTAC | |
| | | AATTCGACCTATCGTGTCGTGAGCGTTCTGACCGTCCTTCACCAA | |
| | | GATTGGCTTAACGGCAAAGAATATAAGTGCAAGGTAAGCAATAA | |
| | | AGCACTTCCGGCCCCAATCGAGAAAACCATTTCCAAGGCCAAAG | |
| | | GTCAACCAAGAGAACCCCAGGTGTATACTCTTCCGCCTTCTCGTG | |
| | | AGGAAATGACTAAAAATCAAGTATCCCTTACGTGTCTGGTTAAAG | |
| | | GTTTTTATCCTAGCGATATTGCTGTTGAATGGGAATCGAACGGTC | |
| | | AGCCGGAGAATAATTATAAAACAACGCCACCCGTCCTGGATAGC | |
| | | GACGGCTCATTTTTTCTGTATAGCAAACTGACTGTAGATAAATCA | |
| | | CGGTGGCAGCAGGGCAATGTATTCAGTTGCTCCGTTATGCATGAA | |
| | | GCGTTACATAATCACTACACGCAGAAATCTCTTAGTCTTTCACCC | |
| | | GGT | |
| Ab- | LC | GAAATTGTGTTGACGCAGTCTCCAGGCACCCTGTCTTTGTCTCCA | 146 |
| 017 | | GGGGAAAGAGCCACCCTCTCCTGCAGGGCCAGTCAGAGTGTTAG | |
| | | CAGCAGCTACTTAGCCTGGTACCAGCAGAAACCTGGCCAGGCTCC | |
| | | CAGGCTCCTCATCTATGGTGCATCCAGCAGGGCCACTGGCATCCC | |
| | | AGACAGGTTCAGTGGCAGTGGGTCTGGGACAGACTTCACTCTCAC | |
| | | CATCAGCAGACTGGAGCCTGAAGATTTTGCAGTGTATTACTGTCA | |
| | | GCAGTATGGTAGCTCACCGCTCACTTTCGGCGGAGGGACCAAAGT | |
| | | GGATATCAAACGTACGGTAGCTGCCCCTTCAGTTTTTATCTTTCCG | |
| | | CCGTCTGACGAGCAGTTAAAATCCGGGACCGCTTCTGTAGTTTGC | |
| | | CTGCTGAATAATTTTTATCCGCGTGAGGCTAAAGTACAATGGAAA | |
| | | GTCGACAATGCTTTGCAGTCGGGAAATTCACAGGAAAGTGTTACG | |
| | | GAGCAGGATTCTAAAGATTCCACATATTCACTCAGCTCCACCCTT | |
| | | ACACTGAGCAAAGCCGACTATGAAAAACATAAAGTTTACGCATG | |
| | | TGAGGTGACGCACCAAGGATTATCCAGTCCGGTCACAAAATCGTT | |
| | | TAACCGCGGTGAGTGT | |

### Example 2

### Characterization of Anti-CD180 Antibodies

### Cell-Based ELISA Assay.

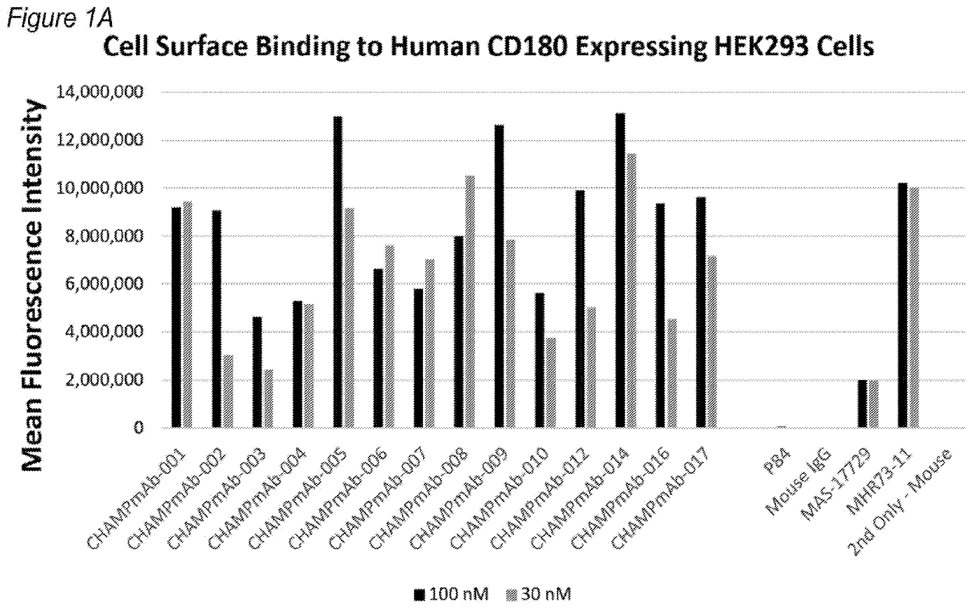

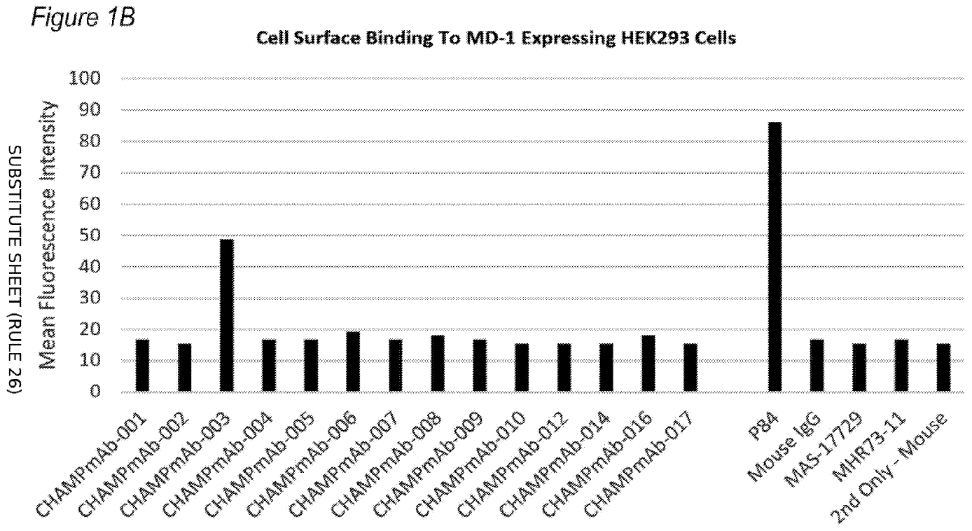

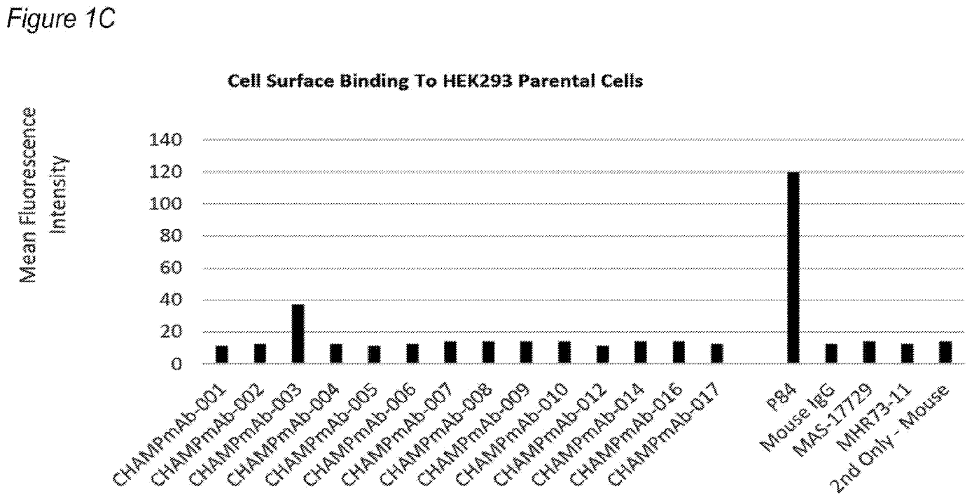

All mAbs were subjected to cell binding screen detected on a flow cytometer. Engineered cells expressing CD180/MD-1, MD-1, or parental HEK cells were used to test for binding of the antibodies. Antibodies at a concentration of 100 nM or 30 nM were incubated with cells at 4° C. for 45 min. R-Phycoerythrin AffiniPure Goat Anti-Human IgG (Jackson Immunoresearch 109-115-098) was used as the secondary antibody. The binding of antibodies on cells were detected on Intellicyt iQue3 VBR. FIG. 1A shows all CHAMP-mAbs had significant binding in the ELISA. The antibodies did not bind to cells expressing MD-1 only (FIG. 1B).

### EC₅₀ Assessment.

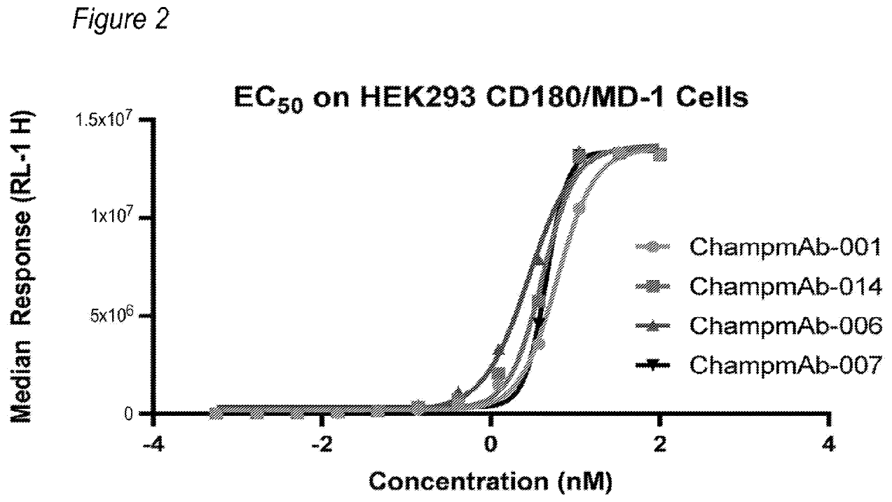

For EC50, anti-CD180 antibodies were tested at a concentration from 100 nM to 0.6 pM (a serial 3-fold dilution) for binding on CD180/MD-1 cells. Cells were then incubated with the secondary antibody R-Phycoerythrin AffiniPure Goat Anti-Human IgG (Jackson Immunoresearch 109-115-098). The data was acquired on Intellicyt iQue3 VBR. Median fluorescence intensities (MFI) were plotted against the concentrations of the anti-CD180 antibodies. EC50 was derived from fitting to 4 parameter dose-response curve. Results in FIG. 2 show the quantitative binding affinity of the antibodies for CD180, demonstrating specific and strong binding.

### Surface Plasmon Resonance (SPR) for Binding Affinity.

Surface Plasmon Resonance (SPR) was used to identify intrinsic binding affinity. Kinetic experiments were performed on Carterra LSA with a running buffer of PBS pH 7.40, 1% BSA, 0.05% Tween20. Antibodies were captured on an anti-human Fc capture chip prepared with a HC30M chip. For kinetics analysis, purified recombinant His-tagged human CD180 from Sino Biologics or from Creative Biomart, or His-tagged rhesus CD180 from Creative Biomart at a concentration from 0.076 nM to 1500 nM (a serial 3-fold dilution) was injected sequentially. For each concentration, there was 5 min association followed by 15 min dissociation. Results were processed and analyzed in Carterra LSA Kinetics Software. The kinetic data was referenced with the interstitial reference spots and double-referenced to a buffer cycle, and then fit globally to a 1:1 binding model to determine their apparent association and dissociation kinetic rate constants (kₒₙ and k_{off} values). The ratio k_{off}/kₒₙ was used to derive the KD value of each antigen/mAb interaction, i.e., KD=k_{off}/kₒₙ.

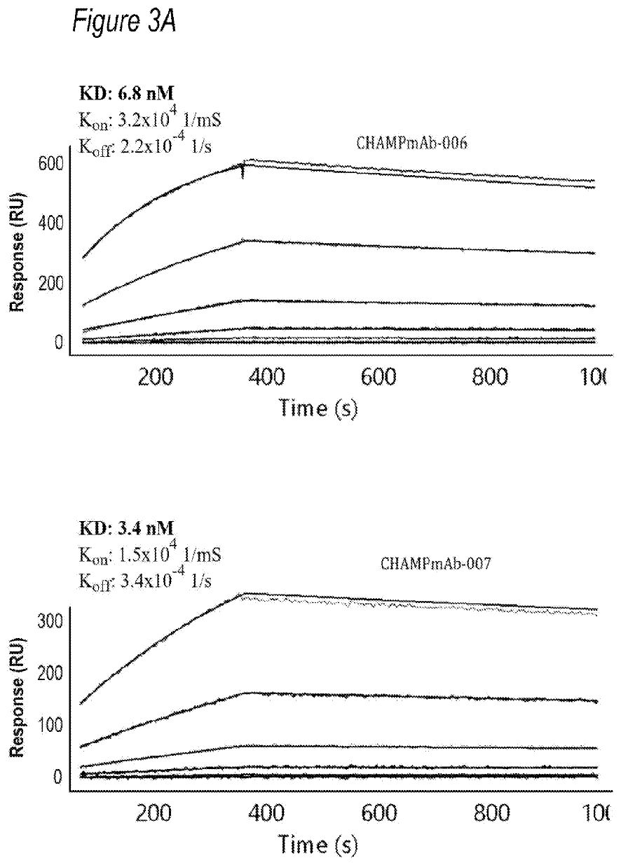

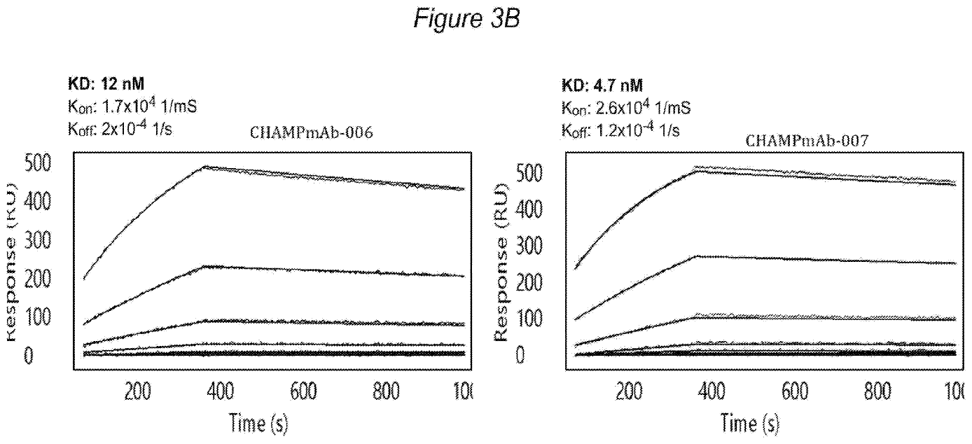

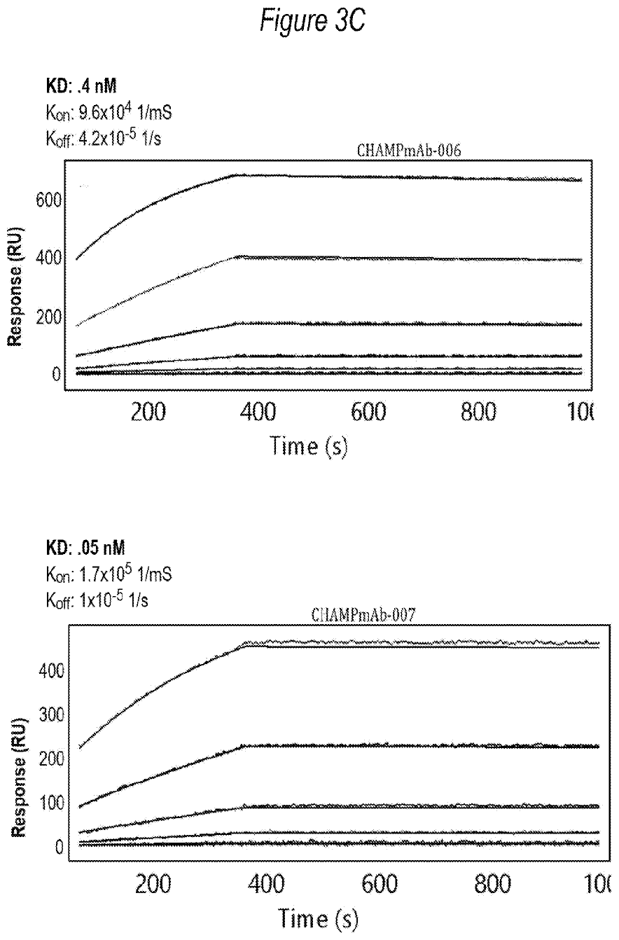

FIGS. 3A-3C show that only Champ mAb-006 and Champ mAb-007 showed binding to soluble CD180 protein, indicating that the epitopes for all other Champ mABs are masked when CD180 is expressed as a soluble protein.

### DSF and Turbidity Assays.

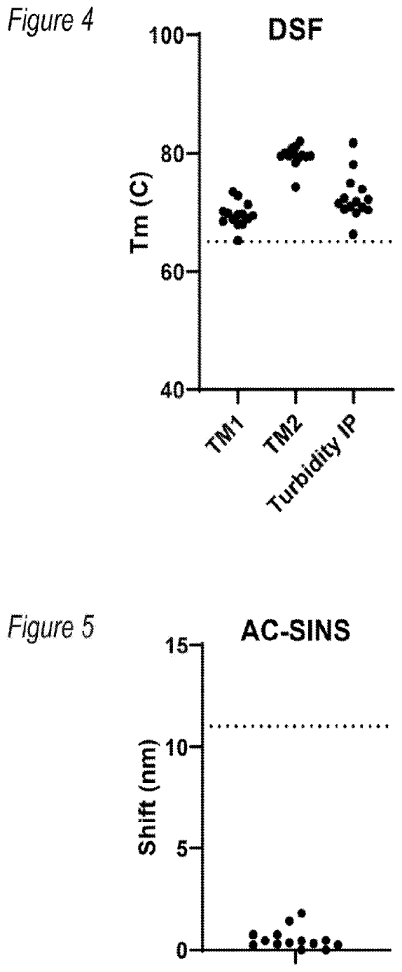

DSF and Turbidity assays were used to determine an antibody's thermal aggregation rate. Thermal stability was assessed via nano differential scanning fluorimetry (nanoDSF) on Prometheus Panta. Each sample was measured in duplicate. Melting temperatures of the antibodies were detected during heating in a linear thermal ramp (0.5° C./min, 25-95° C.). Data was analyzed using the Panta Analysis software. The unfolding transition points were determined from changes in the emission wavelengths of tryptophan fluorescence at 350 and 330 nm. FIG. 4 shows that all the antibodies exhibited an acceptable melting temperature (Tm) above 65° C. Thermal stability is a general biophysical characteristic for an antibody that needs to be accounted for in lead selection. Any lead antibody should have a thermal stability above 65 C. The first peak (TM1) is the denaturing of the VL, the second (TM2) is the VH/CH denaturing.

### Affinity-Capture Self-Interaction Nanoparticle Spectroscopy (AC-SINS) Assay.

AC-SINS was used to test how likely an antibody is to interact with itself. Gold nanoparticles (Ted Pella, 15705-20) were washed with water. Antibody mixture of 80/20 (v/v) capture antibody/non-capture antibodies (Jackson Immuno Research Labs) was buffer exchanges into 20 mM sodium acetate pH 4.5 to a concentration of 500 μg/ml. To prepare 1 ml coated particles, 900 μL of gold nanoparticles were incubated overnight with 100 μL of antibody mixture for 90 min at RT. After antibody coating, thiolated PEG (MW: 2000 Da) were used to quench the beads. The beads were then concentrated 10 fold in PBS. 10 μL 10× concentrated particles solution was incubated with 100 μL of 40 μg/mL of antibody samples on a 384-well polypropylene plate for 2 hrs at room temperature. The plate was then quickly spun down at 3000 rpm and scanned from 510 to 580 nm in increments of 2 nm on Synergy Neo2 Multi-Mode Plate Reader (BioTek). Values reported are averages of duplicate wells and are sample red shift wavelengths at maximum absorbance subtracting the blank reference (PBS only). Greater red shifts indicate increased self-interaction.

FIG. 5 shows that all CHAMP antibodies exhibited a favorable profile below a shift of 11 nM, indicating a low propensity to self-interact.

### Size-Exclusion Chromatography.

Size-exclusion chromatography, also known as molecular sieve chromatography, is a chromatographic method in which molecules in solution are separated by their size, and in some cases molecular weight. Size exclusion chromatography (SEC) was performed with a YMC Diol-200 8×300 mm column (Cat.no #DL20S05-3008WT) on an Agilent 1200 series HPLC instrument. The running buffer was 20 mM sodium phosphate, 400 mM NaCl pH 7.0 at a flow rate of 0.3 mL/min. For freeze-thaw stability, samples frozen at −80 C for 20 minutes, then thawed at room temperature for approximately 20 minutes.

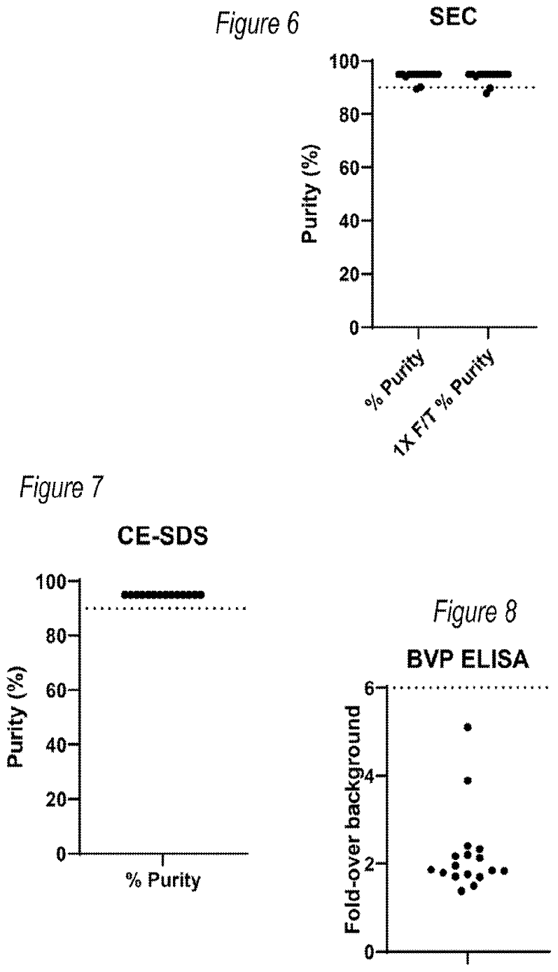

FIG. 6 shows that all CHAMP antibodies show a high level of purity after production (>95%) before and after a freeze thaw cycle (1×F/T).

### Capillary Electrophoresis Sodium Dodecyl Sulfate (CE-SDS).

CE-SDS is an analytical method used to assess the purity of proteins. CE-SDS employed Protein Express Assay Reagent Kit (Perkin Elmer, #CLS960008) and was performed on a LapChip GX II instrument using Protein Express 200 (Perkin Elmer, #760499). The reagents and chip were prepared according to the manufacturer's instruction. Briefly, the reducing sample buffer was prepared by mixing 1 M dichlorodiphenyltrichloroethane with Protein Express Sample Buffer, while the nonreducing buffer consisted of only Protein Express Sample Buffer. Samples were mixed with reducing or non-reducing buffer and denatured at 80° C. for 10 min. Samples were centrifuged at 2,000 g for 1 min to remove air bubbles before placing in the LapChip GXII instrument for analysis.

FIG. 7 shows that all CHAMP mABs tested under this method show a high level of purity (>95%) after production.

### Baculovirus Particles ELISA.

Baculovirus Particles ELISA was used to test the propensity of an antibody to interact with proteins in a non-specific manner. The method was similar as reported by Hotzel et al. (2012). Briefly, baculovirus particles (BVP, Lake Pharma) was diluted 1:100 in 50 mM sodium bicarbonate (pH 9.3). After overnight incubation of 50 μL of BVP on ELISA plates (3369; Corning) at 4° C. overnight, unbound BVPs were aspirated from the wells. All remaining steps were performed at room temperature. The plate was blocked with 100 μL of blocking buffer (PBS with 1% BSA) for 1 h before three washes with 100 μL of PBS. Next, 50 μL of 16 nM testing antibodies was added to the wells and incubated for 1 h followed by washes with 100 μL of PBS. HRP-conjugated goat anti-human IgG antibody at 1:1000 (Jackson ImmunoResearch) was used as the secondary antibody, and incubated for 1 h followed by washes as before. Finally, 50 μL of TMB substrate (34021; Fisher Scientific) was added to each well and incubated for 10-15 min. The reactions were stopped by adding 50 μL of 2 M sulfuric acid to each well. The absorbance was read at 450 nm and BVP score determined by normalizing absorbance by control wells with no test antibody.

FIG. 8 shows that all CHAMP mAbs show a low propensity for non-specific, polyreactive binding.

### Example 3

### Antibody Drug Conjugates (ADC)

Antibody-drug conjugate (ADC) is a new emerging class of highly potent pharmaceutical drugs, which is a great combination of chemotherapy and immunotherapy. The concept of ADC was first presented by the German physician and scientist Paul Ehrlich almost 100 years ago. He described the antibody as a “magic bullet” that identifies their target themselves without harming the organism. Ehrlich also anticipates attaching toxin to the antibodies to improve their therapeutic specificity.

Currently, antibody-drug conjugates (ADCs) are one of the fastest growing anticancer drugs. In one embodiment, this approach comprises a monoclonal antibody (mAb) conjugated to a cytotoxic payload via a chemical linker. ADCs selectively target specific antigens on cancer cells with highly potent cytotoxic agents that offer maximum efficacy while minimizing systemic toxicities. Selection of an appropriate target, mAb, cytotoxic payload, and the manner in which the antibody is linked to the payload are key determinants of the safety and efficacy of ADCs.

Tesirine (SG3249) is an antibody-drug conjugate pyrrolobenzodiazepine (PBD) dimer payload. Tesirine combines potent antitumor activity with desirable physicochemical properties such as favorable hydrophobicity and improved conjugation characteristics. SG3199 is the released warhead component of the ADC payload tesirine. SG3199 retains picomolar activity in a panel of cancer cell lines. PBD dimers are highly efficient DNA minor groove cross-linking agents with potent cytotoxicity.

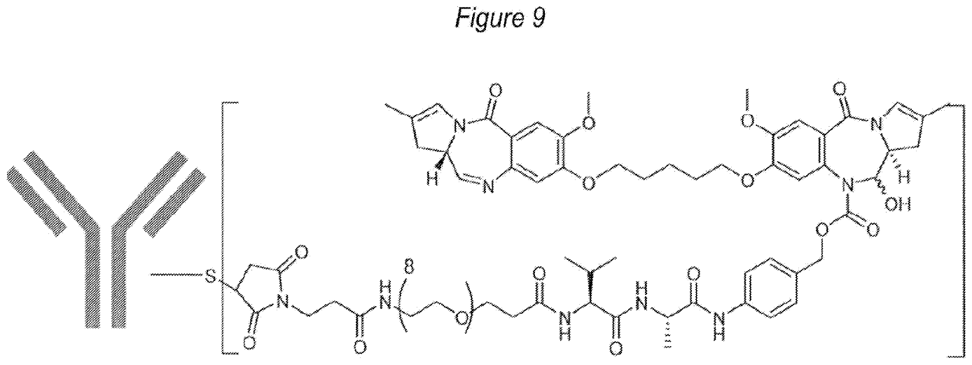

In one embodiment, Ab-001 and Ab-014 hIgG1 monoclonal antibodies as well as a negative control IgG1 mAb was conjugated to the linker-payload tesirine via stochastic maleimide conjugation to interchain cysteines to create CO-ADC-001 and CO-ADC-002 (FIG. 9). Non-specific drug conjugation on naturally available reduced interchain cysteine residues is used to construct the chemical links between the antibodies and payloads. The interchain cysteine residues are reduced followed by the addition of tesirine and cysteine rebridging. Size exclusion chromatography was performed to confirm the purity of monomer ADC (data not shown).

One of ordinary skill in the art would readily incorporate the anti-CD180 binding molecules disclosed herein into ADCs comprising different linkers and/or payloads. Anti-CD180 ADCs comprising tesirine are presented herein as one example. Other linkers for ADC include, but are not limited to, valine-citruline-PAB or valine-alanine-PAB. Other payloads for ADC include, but are not limited to, monomethyl auristatin E (MMAE), maytansine, or calicheamicin. Examples of linker payload combination include, but are not limited to, tesirine, deruxtecan, ozogamicin, or emtansine.

The ADCs disclosed herein (e.g., tesirine conjugated ADC and deruxtecan conjugated ADC) can be used to treat diseases such as, but not limited to, acute myeloid leukemia, mantle cell lymphoma, multiple myeloma. follicular lymphoma, B-Acute lymphoblastic leukemia, or diffuse large B-Cell lymphoma.

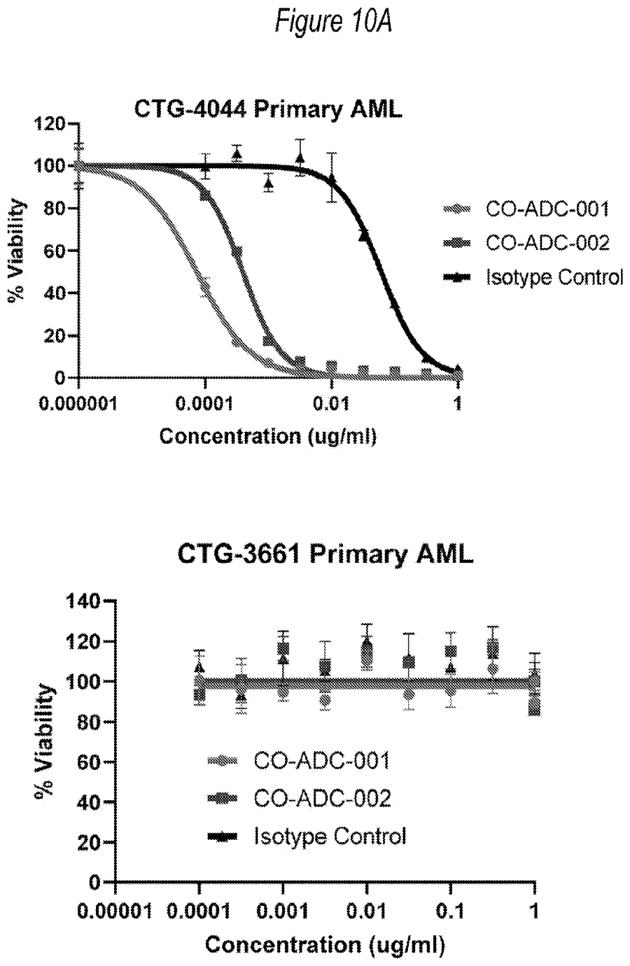

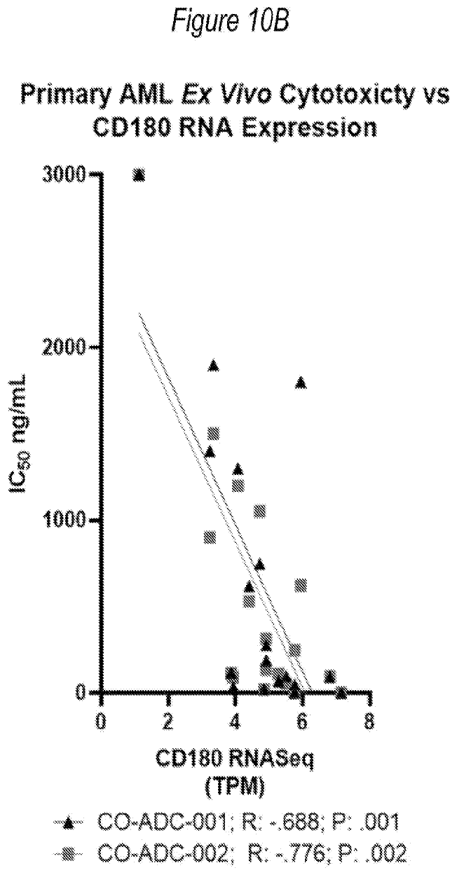

FIGS. 10A-10B show ex vivo cytotoxicity of ADCs in primary acute myeloid leukemia (AML). The results show effective cell killing in a primary model of AML. Cytotoxicity is correlated with CD180 expression; CD180 is the target of the antibody in the ADC. The higher the expression of CD180, the more potent the ADC is.

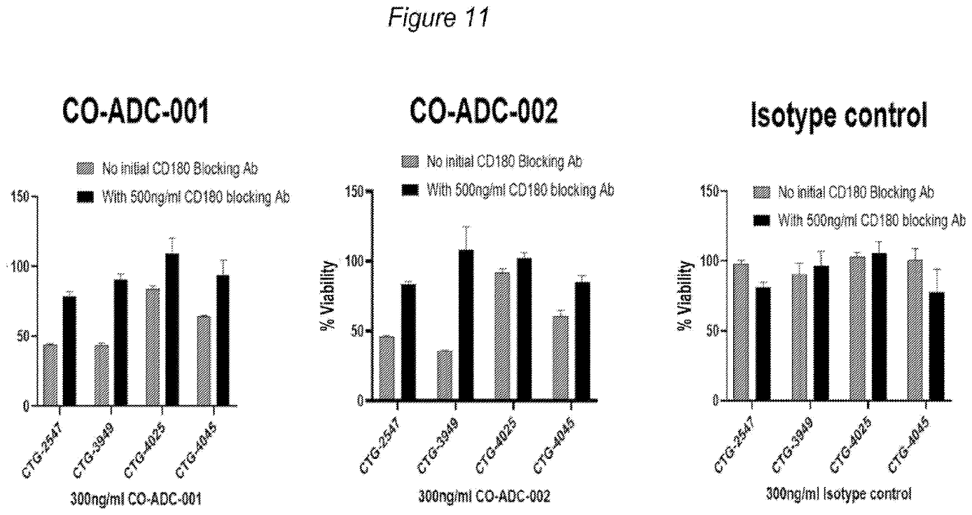

FIG. 11 shows inhibition of ex vivo cytotoxicity of ADCs in primary acute myeloid leukemia. A CD180-targeted naked antibody was added first to block the binding of the CD180-targeted ADC. As a result, no ADC could bind, thereby eliminating any ADC-mediated cell killing. The results show the cell killing is dependent on the ADC binding to CD180.

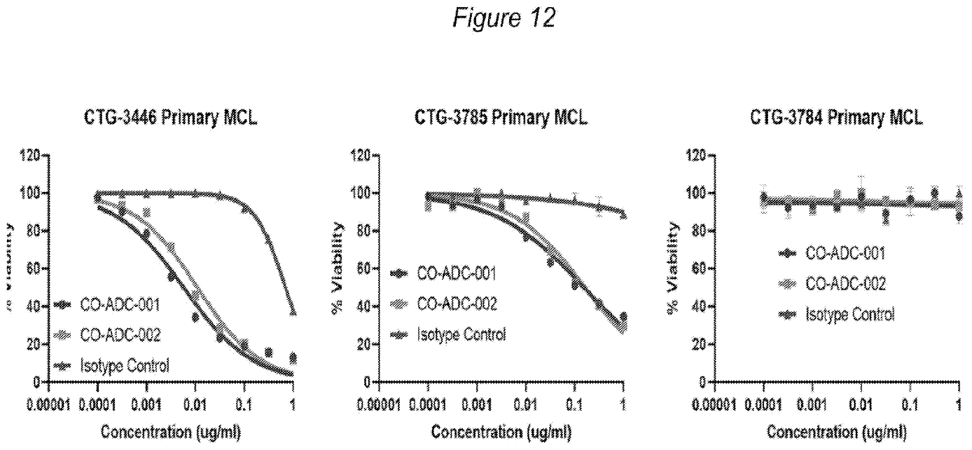

FIG. 12 shows ex vivo cytotoxicity of ADCs in primary mantle cell lymphoma (MCL). The results show effective cell killing in a primary model of MCL.

### Example 4

CD180 Targeting in Primary AML with Anti-CD180 Antibody-Drug Conjugates

CD180 surface expression is enriched in Primary AML over normal immune cells. Thus, CD180-high tumors are the target of therapy with anti-CD180 antibody-drug conjugates according to the present invention.

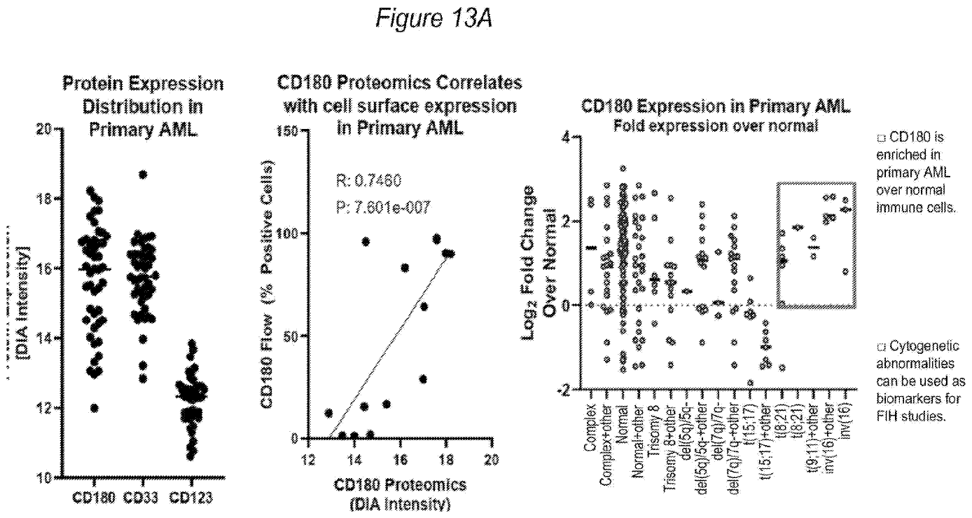

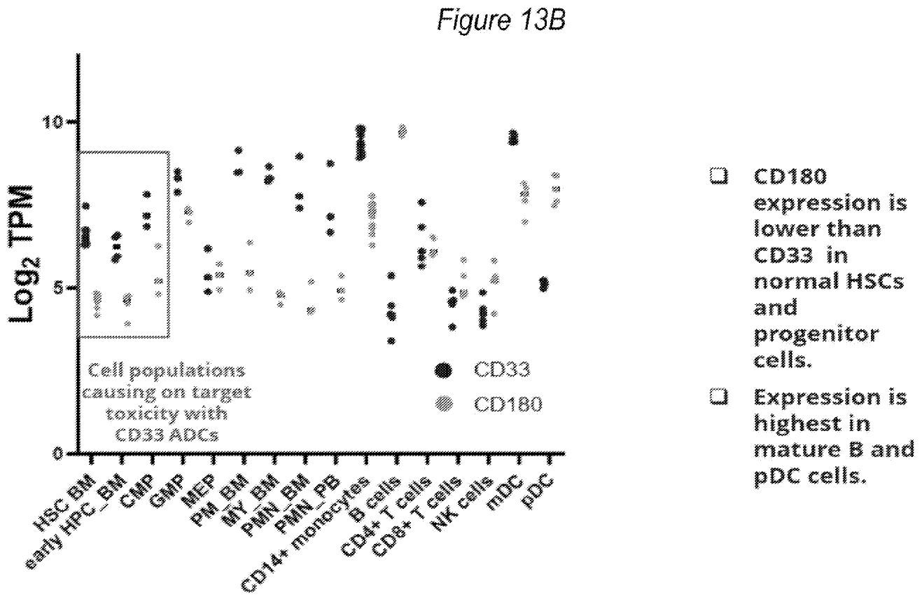

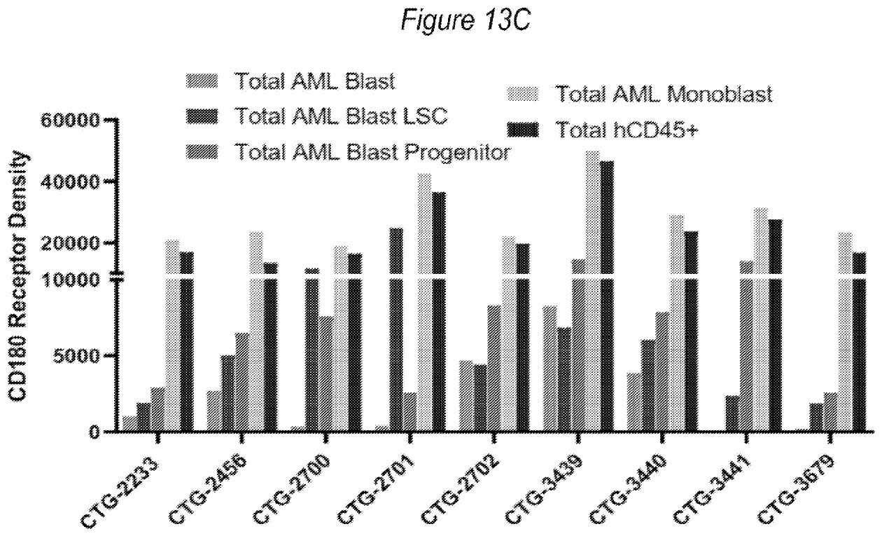

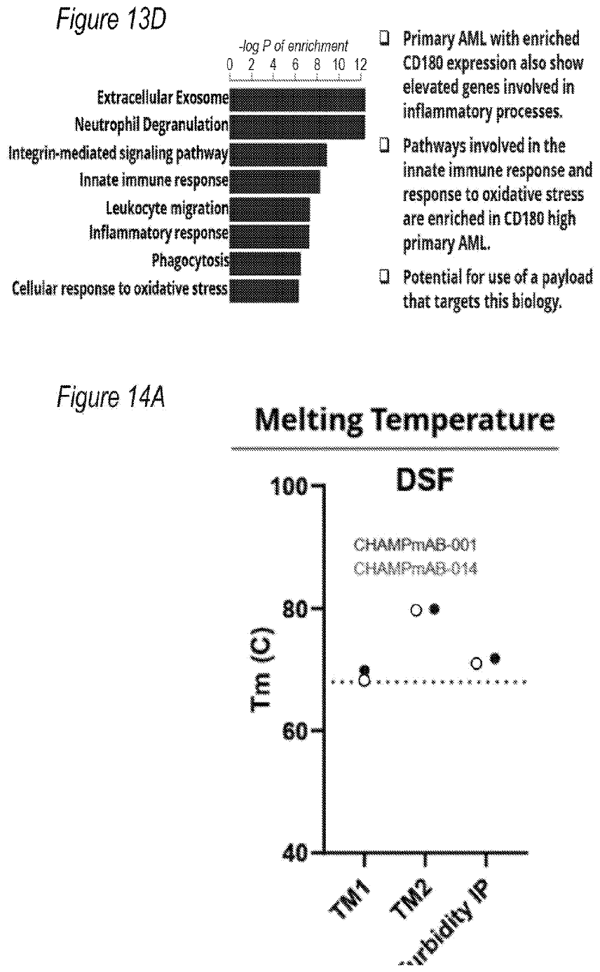

FIGS. 13A-13D show the CD180 target overview. FIG. 13A shows CD180 surface expression is enriched in Primary AML: protein expression distribution in Primary AML CD180 proteomics correlates with cell surface expression in Primary AML, and CD180 is elevated in Primary AML over normal immune cells; thus; cytogenetic abnormalities can be used as biomarkers for first-in-human study (FIH) studies. FIG. 13B shows CD180 expression in Primary normal bone marrow (BM) suggests an improved therapeutic index (TI) over CD133 ADCs. CD180 expression is lower than CD133 in normal hematopoietic stem cells (HSCs) and progenitor cells and expression is highest in mature B and plasmacytoid dendritic (pDC) cells. FIG. 13C shows CD180 is expressed on leukemic stem cells (LSC) and progenitor cells in Primary AML which will eliminate measurable residual disease (MRD). FIG. 13D shows enriched inflammatory signaling is observed in CD180-high tumors. Inflammatory AML provides a survival niche for LSCs. Primary AML with enriched CD180 expression also show elevated genes involved in inflammatory processes. Pathways involved in the innate immune response and response to oxidative stress are enriched in CD180 high primary AML.

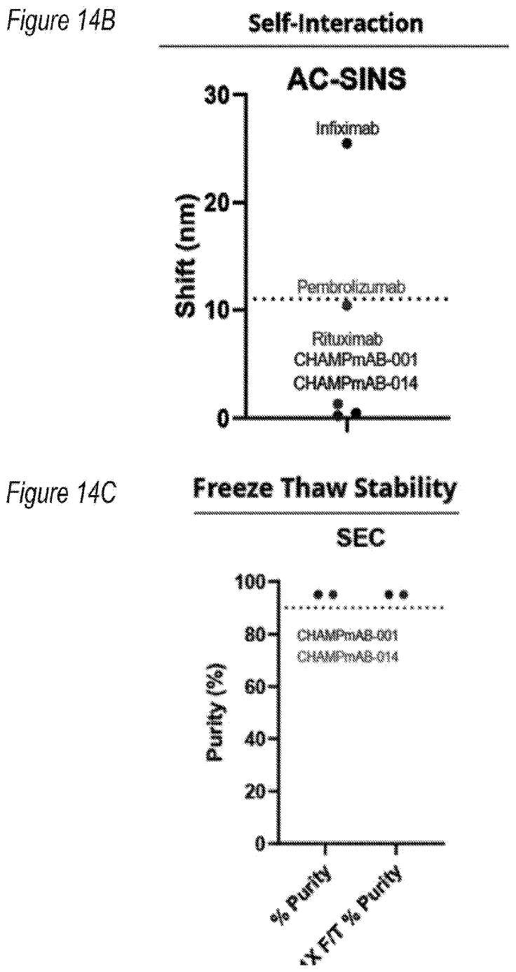

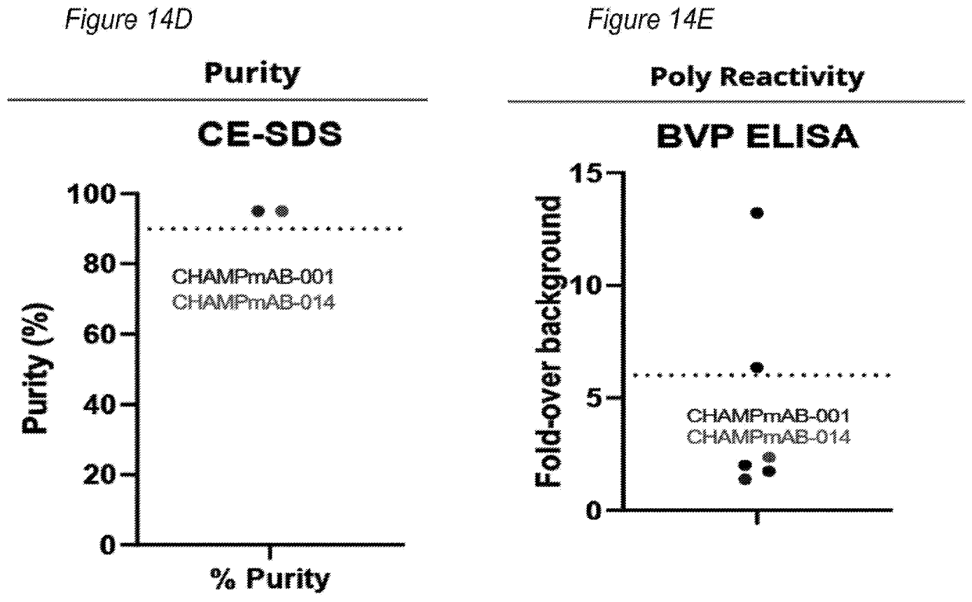

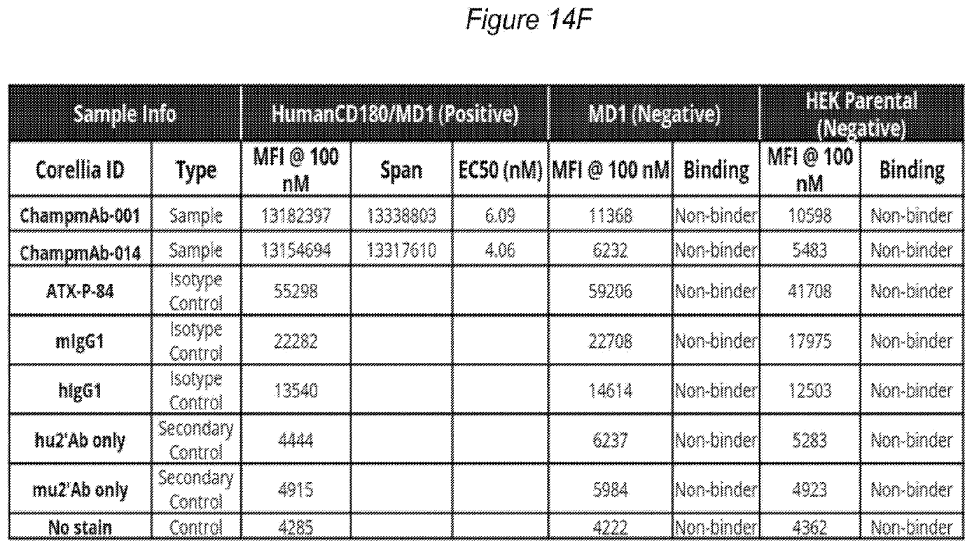

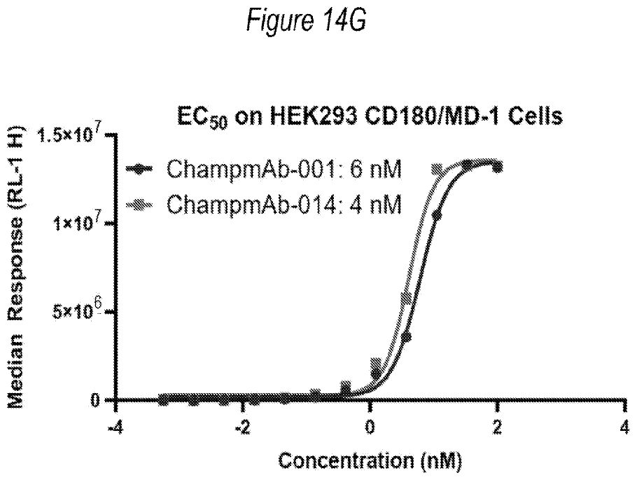

Lead anti-CD180 antibodies, CHAMPmAB-001 and CHAMPmAB-014, were characterized. FIGS. 14A-14F show the melting temperature, self-interaction, freeze thaw stability, purity of anti-CD180 antibody leads CHAMPmAB-001 and CHAMPmAB-014; a comparison of CHAMPmAB-001 and CHAMPmAB-014 with isotype controls (ATX-P-84, and mIgG1), with secondary controls (hIgG1, hu2′Ab only and mu2′Ab only) and with no stain (a control sample) for binding to humanCD180/MD1 (positive) cells, MD1 (negative) cells, and HEK parental cells (negative) (FIG. 14F)

CHAMPmAB-001 (6 nM) and CHAMPmAB-014 (4 nM) half maximal effective concentration (EC₅₀) on HEK293 CD180/MD-1 cells are shown in FIG. 14G.

ADCs, prepared as described in Example 3, were characterized and conjugated to tesirine or deruxtecan.

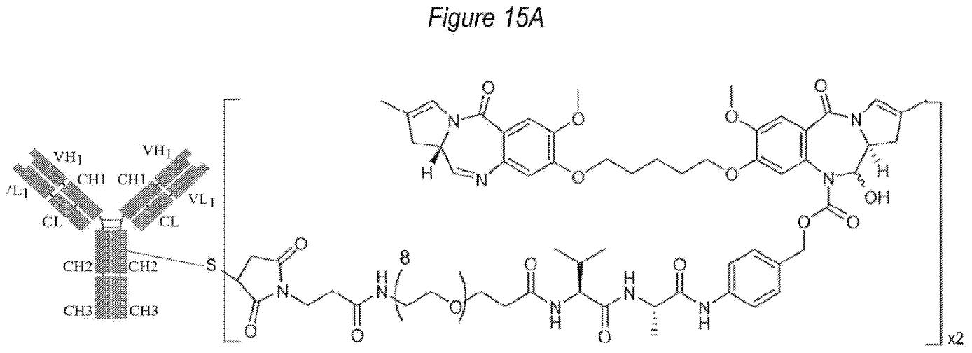

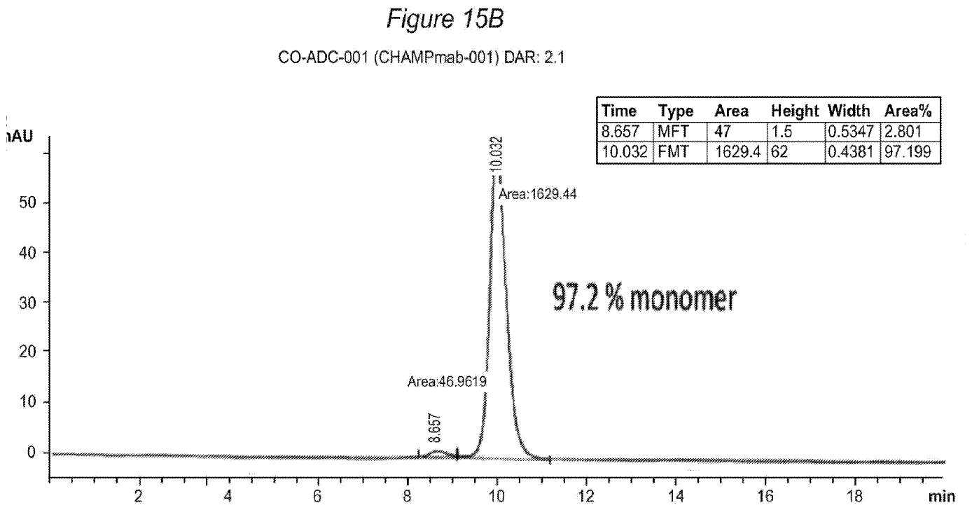

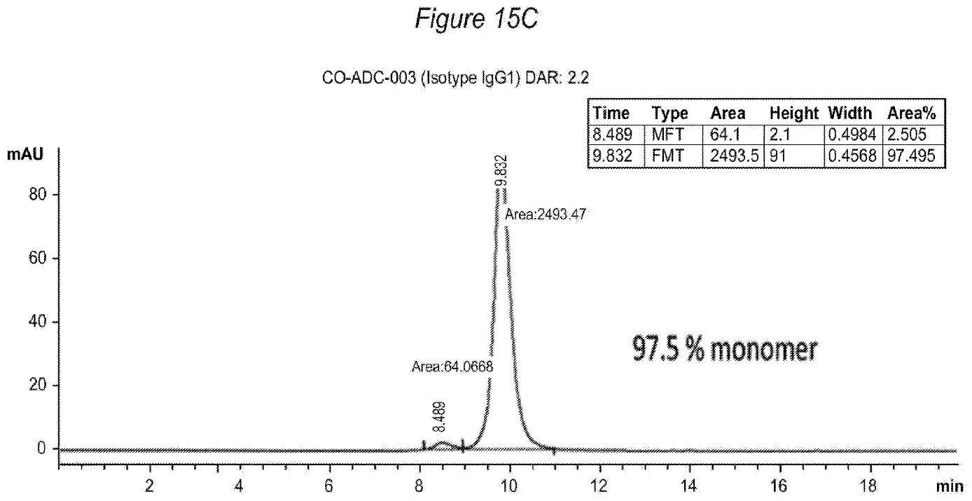

FIGS. 15A-15C show conjugation of anti-CD180 antibody lead CHAMPmAB-001 to tesirine compared to conjugation of an isotype IgG1 to tesirine. FIG. 15A shows a schematic of an embodiment of an ADC of anti-CD180 antibody conjugated to the linker-payload tesirine (×2). FIG. 15B shows a drug-to-antibody ratio (DAR) of 2.1, i.e., the average number of drug conjugated to an anti-CD180 antibody according to the present disclosure, CHAMPmAB-001, conjugated to the linker-payload tesirine (×2) (CO-ADC-001). FIG. 15C shows a DAR of 2.2 of an isotype IgG1 conjugated to the linker-payload tesirine (×2) (CO-ADC-003).

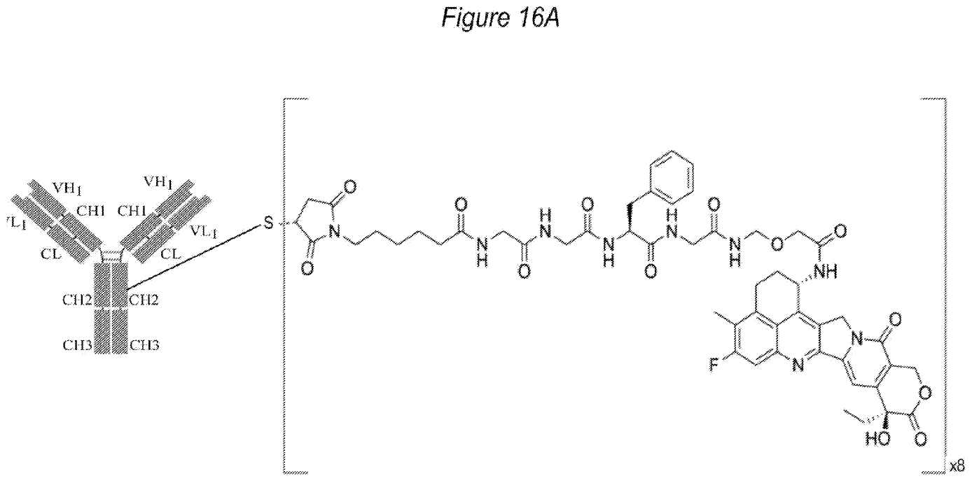

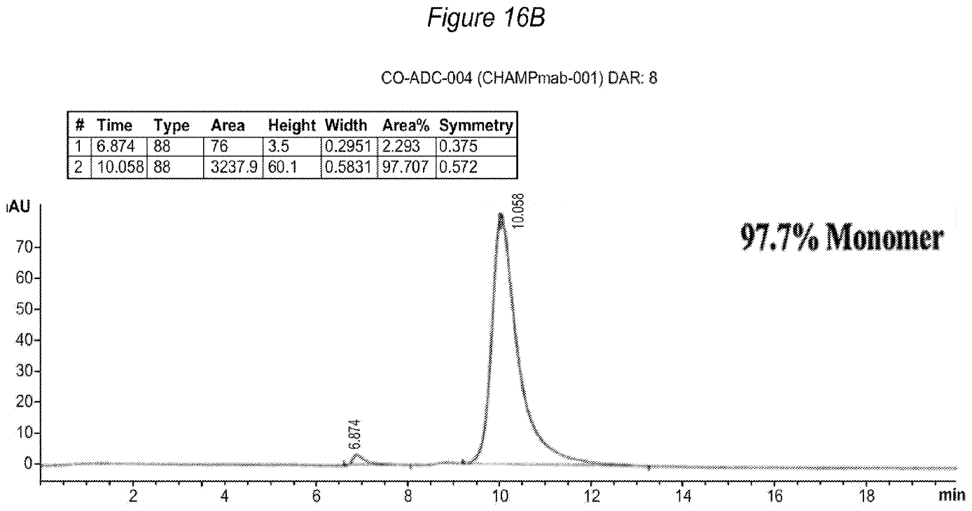

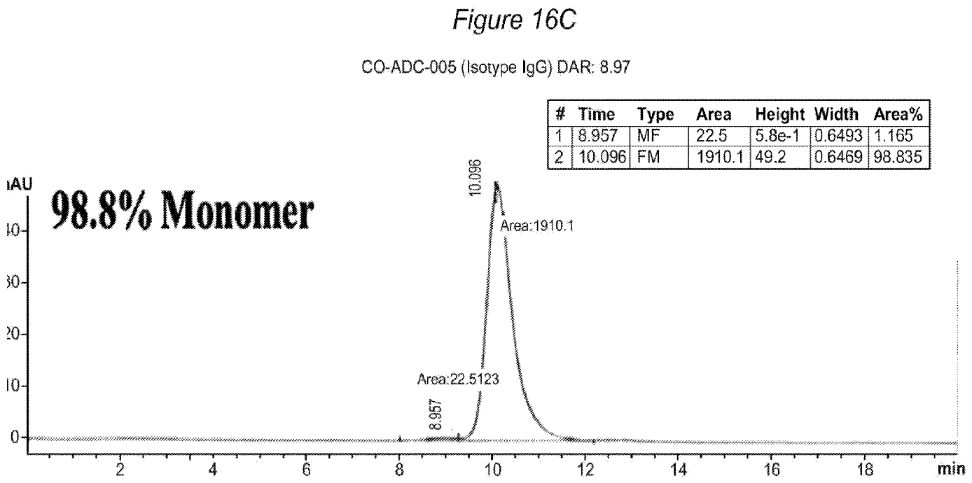

FIGS. 16A-16C show conjugation of anti-CD180 antibody lead CHAMPmAB-004 to deruxtecan compared to conjugation of an isotype IgG1 to deruxtecan. FIG. 16A shows a schematic of an embodiment of an anti-CD180 antibody conjugated to the linker-payload deruxtecan (×8). FIG. 16B shows a DAR of 8 of an anti-CD180 antibody according to the present disclosure, CHAMPmAB-001, conjugated to the linker-payload deruxtecan (×8). FIG. 16C shows a DAR of 8.97 of an isotype IgG1 conjugated to the linker-payload deruxtecan (×8).

### Example 5

### Potent CD180-Dependent Cytotoxicity of Anti-CD180 ADC was Observed in Primary AML Tumors

The cytotoxicity of an anti-CD180 ADC CO-ADC-001, a tesirine (DAR 2) conjugated therapeutic mAb, and an isotype control ADC was compared in tumors from two Primary AML (CTG-4044 and CTG-2240) and measured as % viability. The ADC CO-ADC-001 was observed to have a potent CD180-dependent cytotoxicity compared to the isotype control ADC.

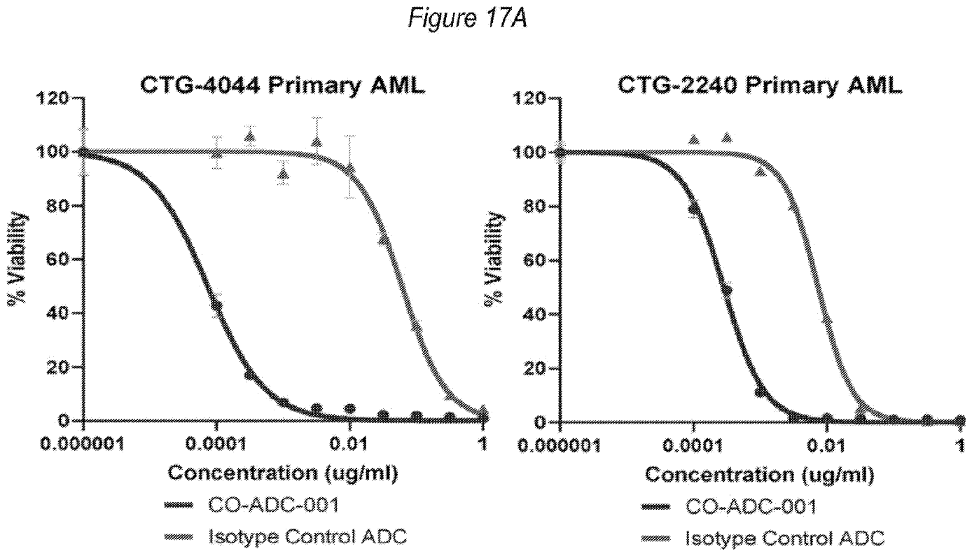

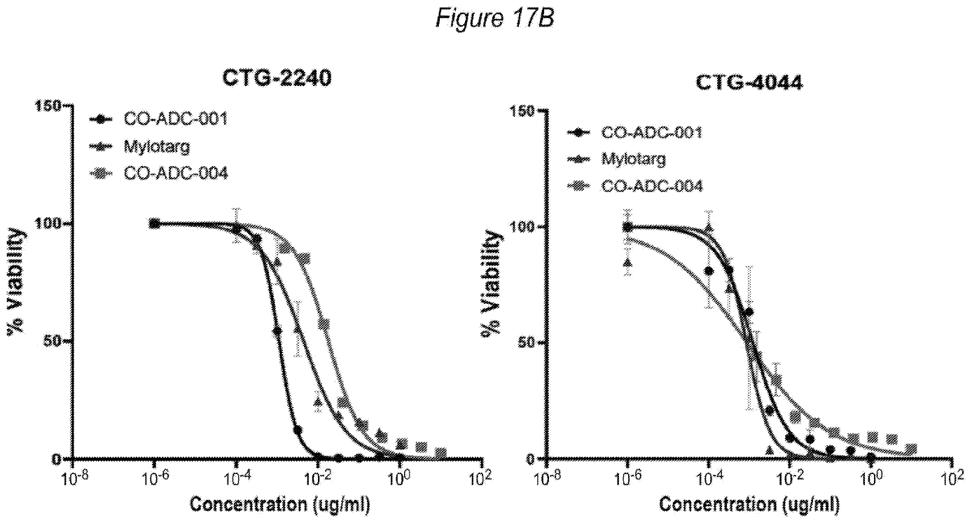

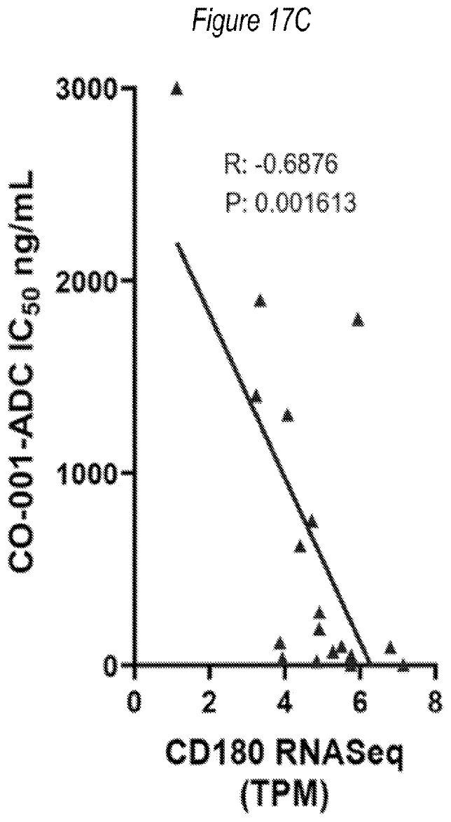

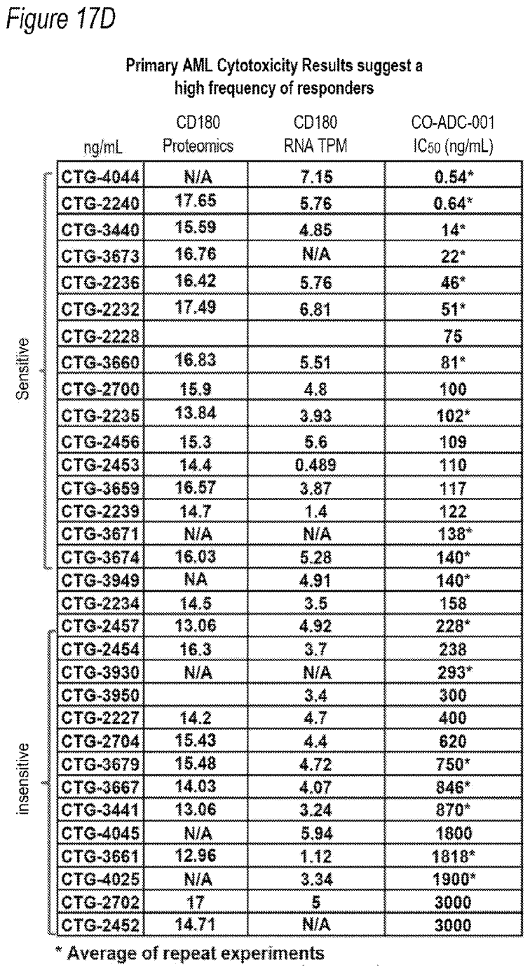

FIG. 17A shows that tesirine conjugates are potent ADCs in CTG-2240 Primary AML: CO-ADC-001 is a tesirine (DAR 2) conjugated therapeutic anti-CD180 mAb. FIG. 17B shows that deruxtecan conjugates are also potent ADCs in CTG-2240 Primary AML and are comparable with MYLOTARG® (gemtuzumab ozogamicin, which is indicated for the treatment of newly diagnosed CD33-positive AML in adults and pediatric patients 1 month and older and in relapsed or refractory CD33-positive AML in adults and pediatric patients 2 years and older).

CO-ADC-004 is a deruxtecan (DAR 8) conjugated therapeutic anti-CD180 mAb. FIG. 17C shows that CO-ADC-001 cytotoxicity correlates with CD180 expression in Primary AML. FIG. 17D shows that Primary AML cytotoxicity results suggest a high frequency of responders.

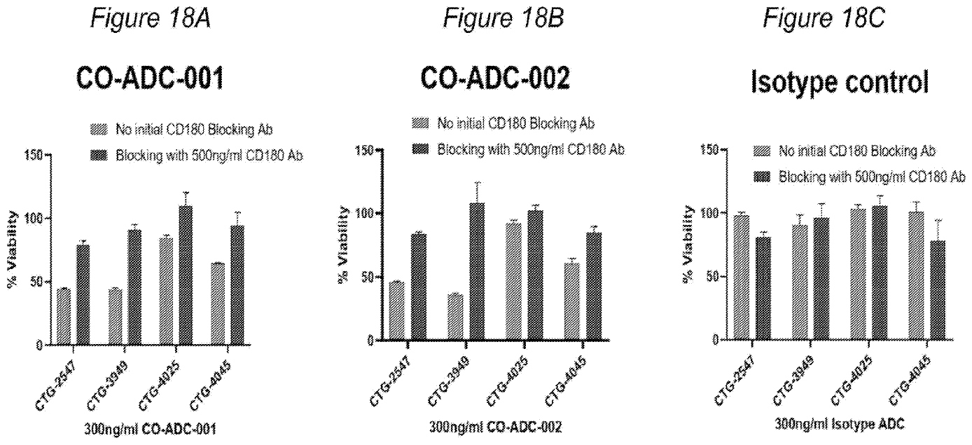

FIGS. 18A-18C show that blocking CD180 with naked CD180 mAb reduces ADC activity. FIG. 18A shows the % viability of primary AML after addition of CO-ADC-001 (300 ng/ml) with no initial CD180 blocking Ab compared to the % viability of the primary AML after addition of CD180 ADCs when naked CD180 antibody 500 ng/ml was added prior to the addition of CD180 ADCs; the naked antibody blocked ADC binding to CD180 to CD180. FIG. 18B shows the % viability of primary AML after addition of CO-ADC-002 (300 ng/ml) with no initial CD180 blocking Ab compared to the % viability of the primary AML after addition of CD180 ADCs when naked CD180 antibody (500 ng/ml) was added prior to the addition of CD180 ADCs, which blocked ADC binding to CD180. FIG. 18C shows the % viability of primary AML after addition of isotype ADC (300 ng/ml) with no initial CD180 blocking Ab compared to the % viability of the primary AML after addition of isotype ADCs when naked CD180 antibody (500 ng/ml) was added prior to the addition of CD180 ADCs, which blocked the isotype ADC binding to CD180.

### Example 6

### Potent CD180-Dependent Cytotoxicity of Anti-CD180 ADC was Observed in Primary MCL Tumors

The cytotoxicity of an anti-CD180 ADC CO-ADC-001, a tesirine (DAR 2) conjugated therapeutic mAb, and CO-ADC-003, a tesirine conjugated negative control mAb, was compared in tumors from three Primary MCL (CTG-3446, CTG-3785 and CTG-3448) and measured as % viability. The ADC CO-ADC-001 was observed to have a potent CD180-dependent cytotoxicity compared to the tesirine conjugated negative control mAb.

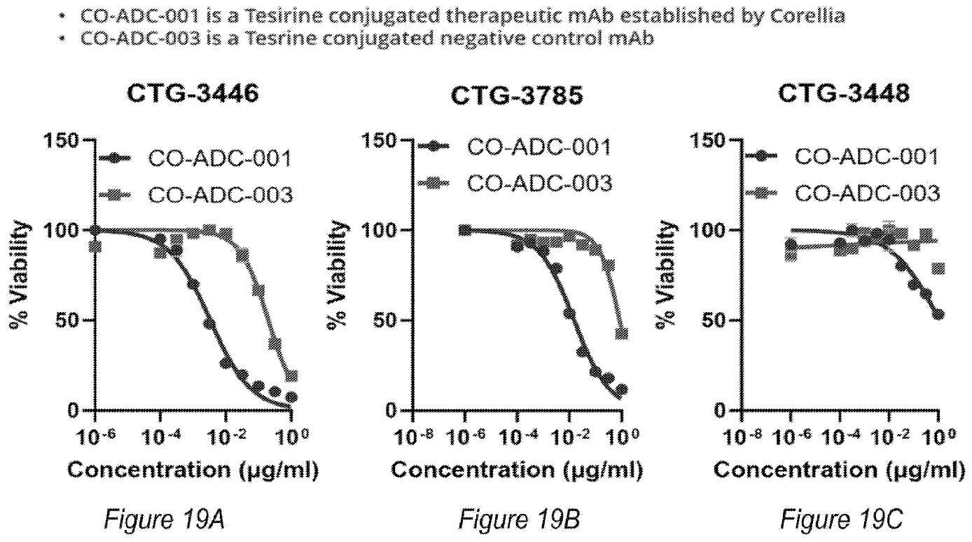

FIGS. 19A-19C show that CD180 ADCs are potent against Primary MCL. FIG. 19A shows the % viability of primary MCL with CO-ADC-001, a tesirine conjugated therapeutic anti-CD180 mAb, compared to the % viability of primary MCL with CO-ADC-003, a tesirine conjugated negative control mAb. FIG. 19A shows the % viability of Primary MCL CTG-3446 with CO-ADC-001 and CO-ADC-003, respectively. FIG. 19B shows the % viability of Primary MCL CTG-3785 with CO-ADC-001 and CO-ADC-003, respectively. FIG. 19C shows the % viability of Primary MCL CTG-3448 with CO-ADC-001 and CO-ADC-003, respectively.

### Example 7

### CD180 ADCs Are Very Potent Against Disseminated Primary AML In Vivo

CTG-2240 Primary AML tumor cells were injected into mice by TVI (Tail Vein Injection) for engraftment of bone marrow, spleen, and peripheral blood to produce disseminated primary AML. Animals were randomized when bone marrow reached a concentration of 20% AML.

CO-ADC-004 (anti-CD180 antibody CO-mAb-020 conjugated to deruxtecan) was administered as a single dose of 5 MKP (mg/kg) by I.V. injection. The endpoint was assessed via flow cytometry in whole blood and bone marrow.

ADC CO-ADC-004 was administered as a single dose of 5 MKP (mg/kg) by I.V. injection. The endpoint was assessed via flow cytometry in bone marrow.

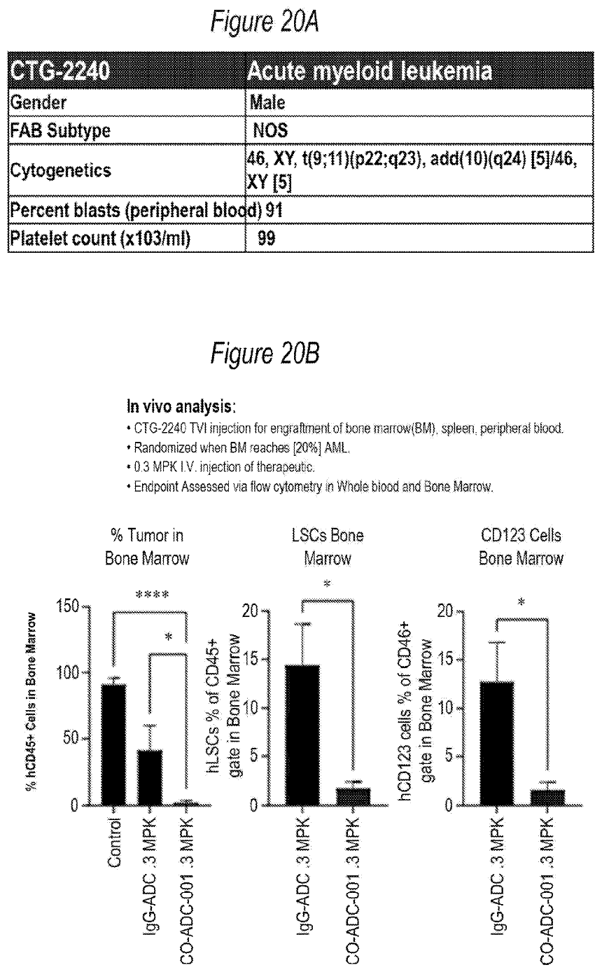

FIGS. 20A-20B show that CO-ADC-001, a CD180-targeted DAR2 tesirine conjugate (anti-CD180 antibody CO-mAb-020 conjugated to tesirine), is very active against disseminated primary AML in vivo. FIG. 20A shows the characteristics of CTG-2240 Primary AML. FIG. 20B shows results of in vivo analysis of % tumor in bone marrow, LSCs in bone marrow and CD123 cells in bone marrow after administration of CO-ADC-001 as a single dose of 0.3 MPK (mg/kg) I.V. injection compared to 0.3 MPK control and IgG-ADC as a single dose of 0.3 MPK.

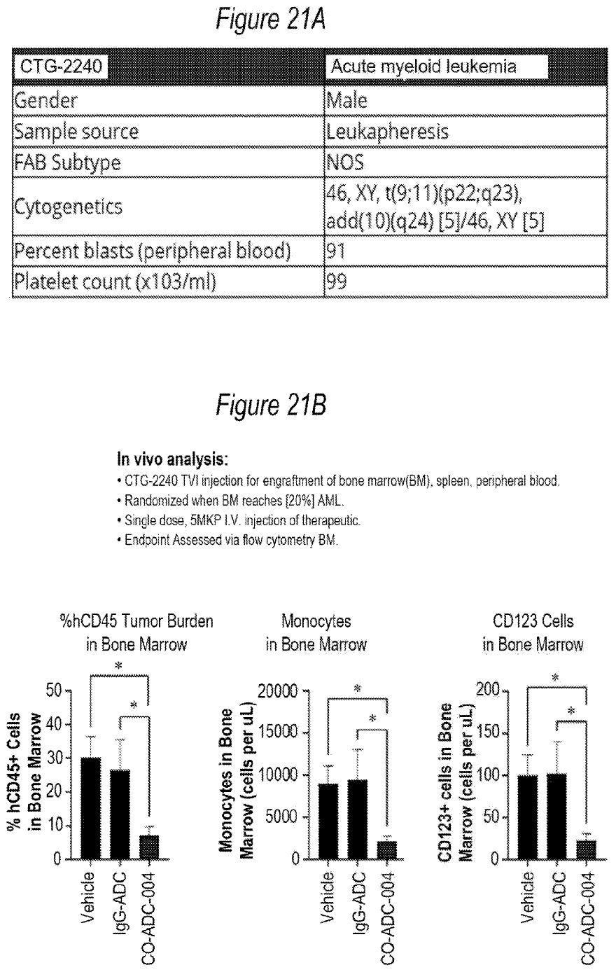

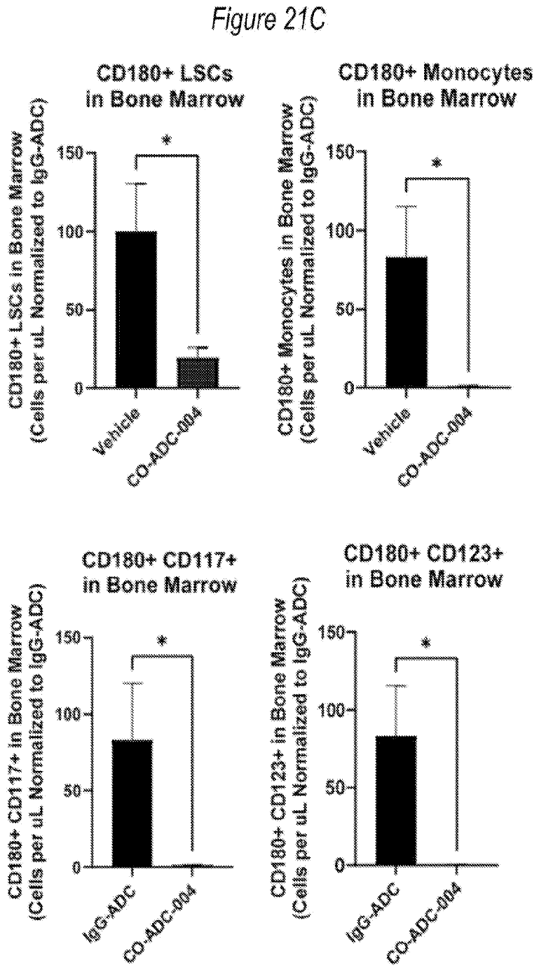

FIGS. 21A-21C show that ADC CO-ADC-004, a CD180-targeted DAR8 deruxtecan conjugate, is very active against disseminated primary AML in vivo. FIG. 21A shows the characteristics of CTG-2240 Primary AML. FIG. 21B shows results of in vivo analysis of % hCD45 tumor in bone marrow, monocytes in bone marrow, CD123 cells in bone marrow, after administration of 5 MPK CO-ADC-004 compared to control (vehicle) and 5 MPK IgG-ADC (CO-ADC-005), as well as CD180+ LSCs in bone marrow, CD180+ monocytes in bone marrow, CD180+CD117+ in bone marrow, and CD180+CD123+ in bone marrow after administration of IgG-ADC or CO-ADC-004, an exemplary embodiment according to the present invention.

FIG. 21C shows results of in vivo analysis of % CD180+ LSCs in bone marrow, % CD180+ monocytes in bone marrow, CD180+CD117+ in bone marrow and CD180+CD123+ in bone marrow.

### Example 8

### Cellular Binding of CD180 ADCs to HEK293 CD180/MD-1 Cells and Potent Anti-Tumor Effectiveness In Vivo

Anti-CD180 antibody CO-mAb-020 (also known as Ab-001 and CHAMPmAb-001) is a fully human IgG1, whose HCDR1, HCDR2 and HCDR3 (SEQ ID NOS: 1-3) and LCDR1, LCDR2 and LCDR3 (SEQ ID NOS: 4-6) are shown in Tables 1-2, respectively, supra. The amino acid sequences of VH, VL, HC and LC of anti-CD180 antibody CO-mAb-020 are shown in Table 3 (SEQ ID NOS: 7, 8 and 11). The nucleotide sequences encoding the VH, VL HC and LC of anti-CD180 antibody CO-mAb-020 are shown in Table 4 (SEQ ID NOS: 9, 13 and 14).

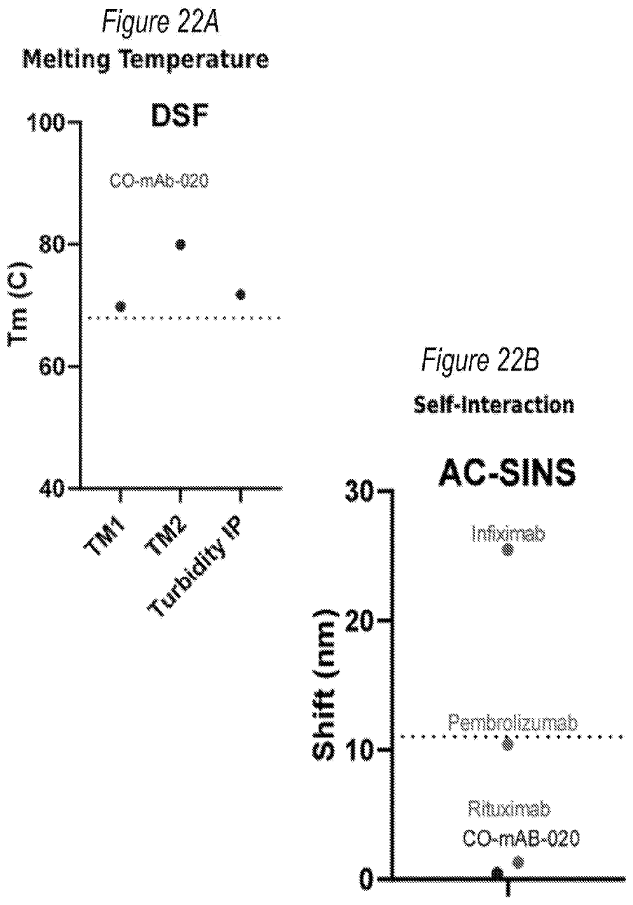

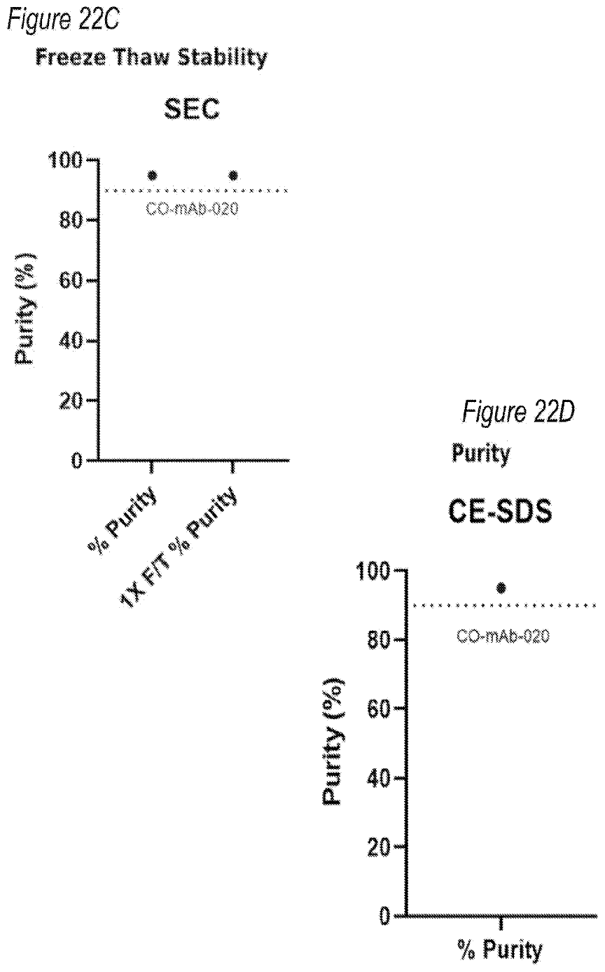

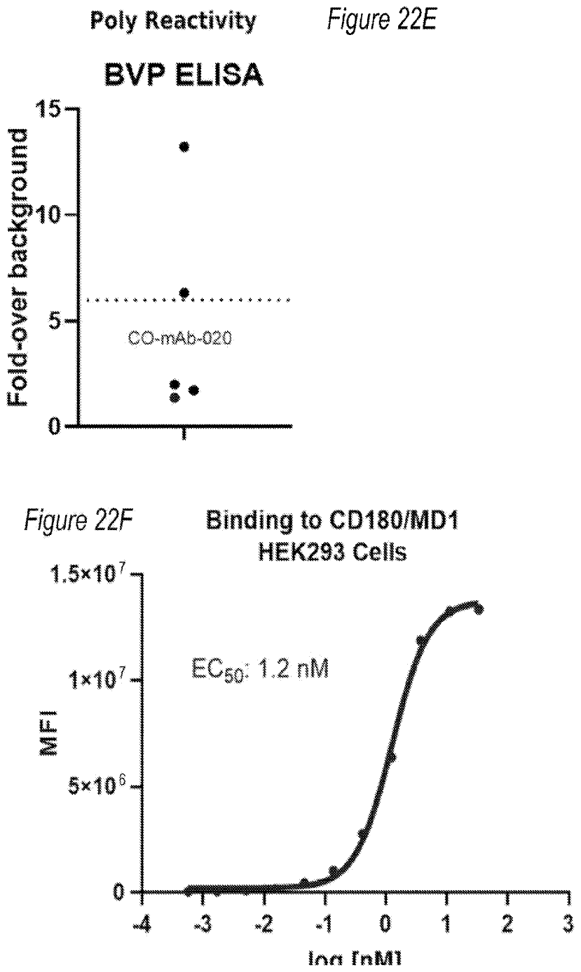

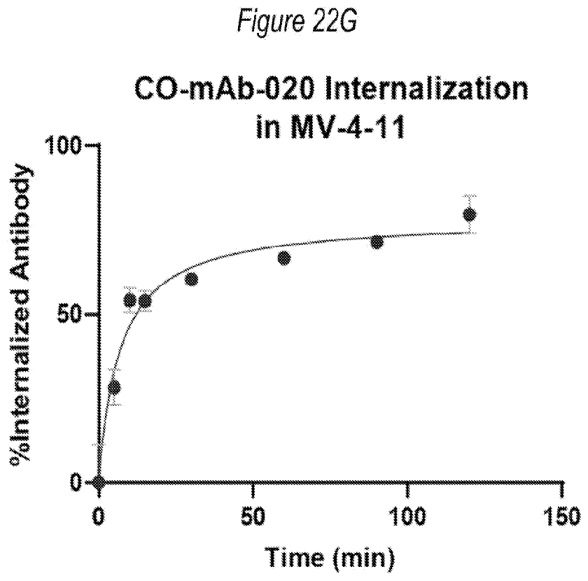

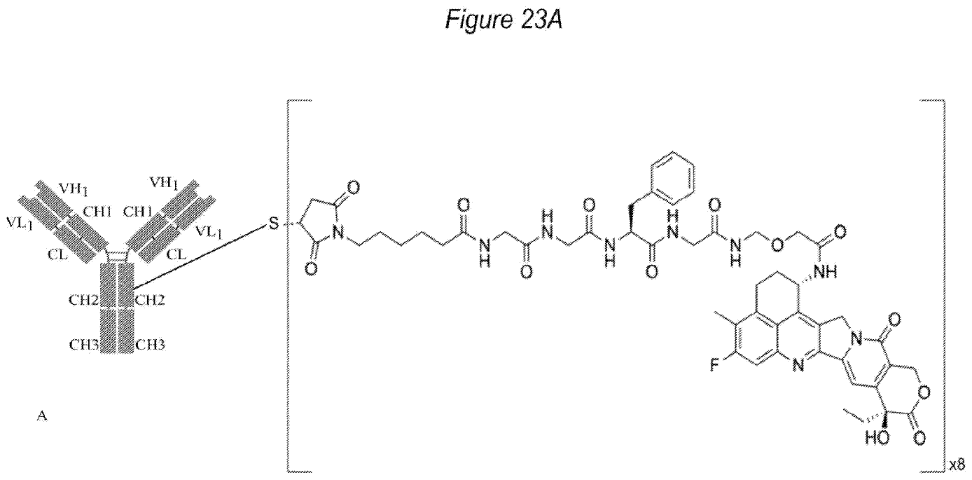

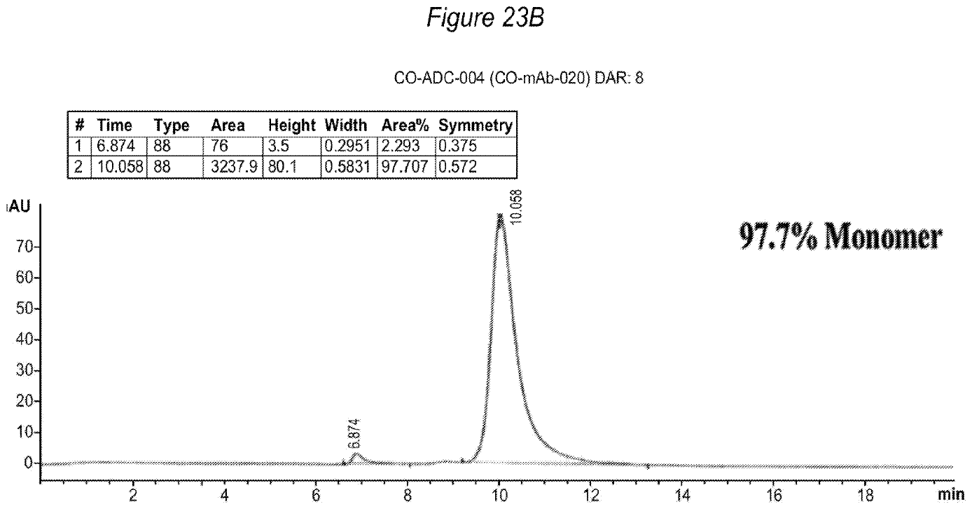

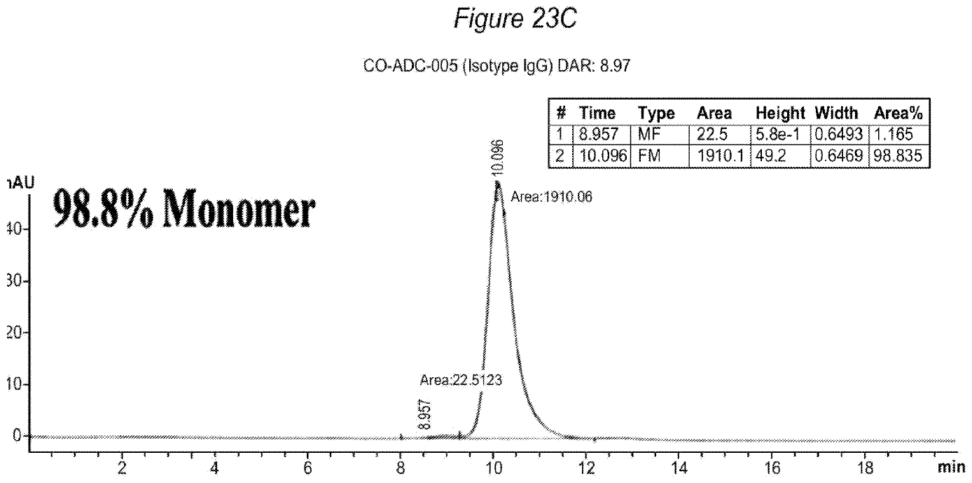

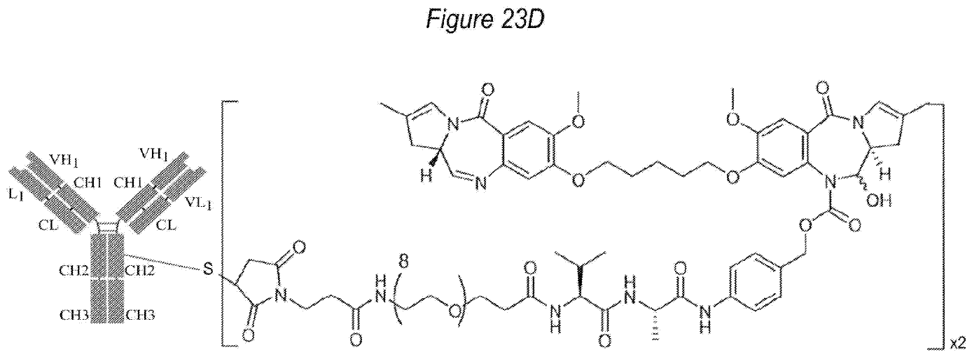

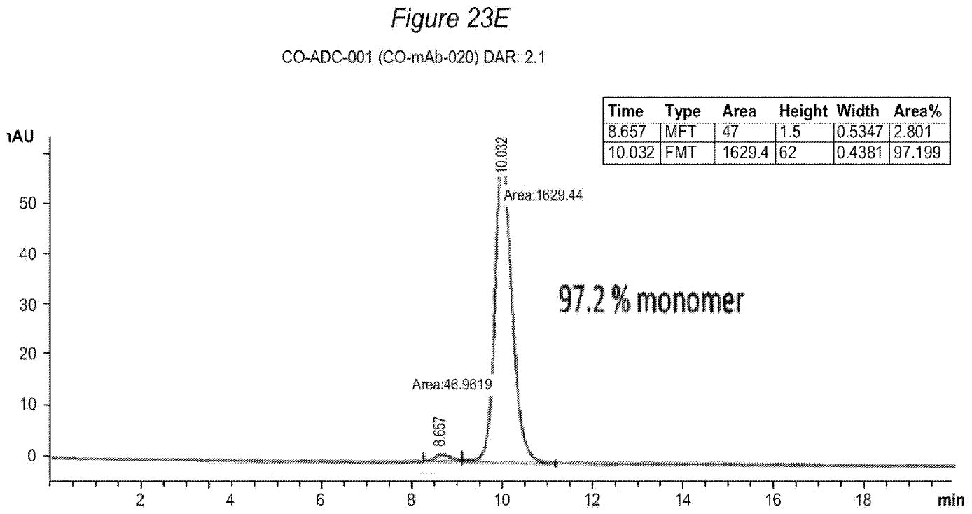

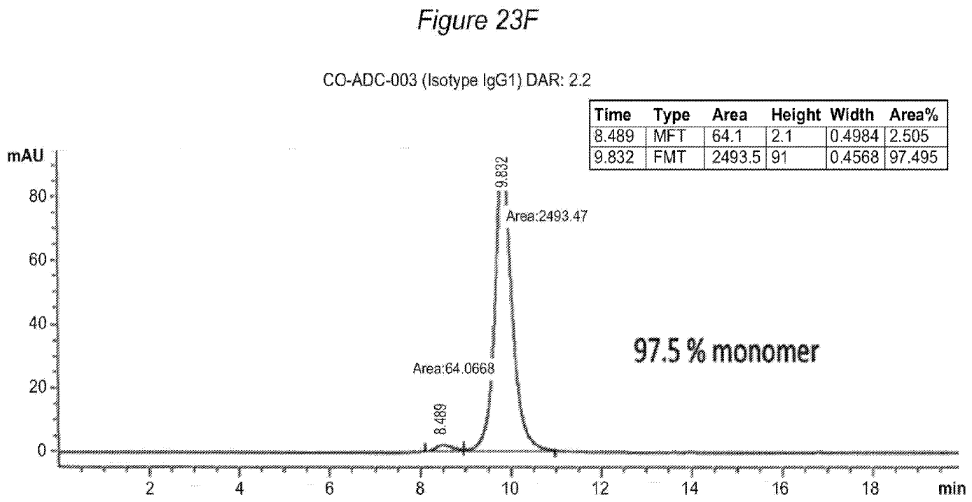

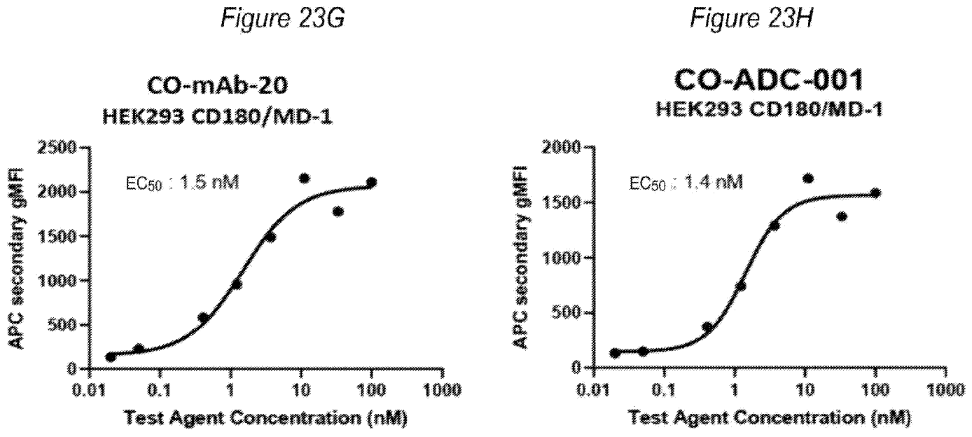

The properties of melting temperature, self-interaction, freeze thaw stability, purity, poly reactivity, binding to CD180/MD1 HEK293 cells, and CO-mAb-020 internalization in MV-4-11 of anti-CD180 antibody CO-mAb-020, are shown in FIGS. 22A-22G. Anti-CD180 antibody CO-mAb-20 conjugated to either deruxtecan (CO-ADC-004) DAR: 8 (FIG. 23B) or to tesirine (CO-ADC-001) DAR: 2.1 (FIG. 23E), respectively, were subjected to EC50 assessment via cell-based ELISA using HEK293 cells expressing human CD180/MD-1. As shown in FIGS. 23G-23H, cellular binding to HEK293 CD180/MD-1 cells is unchanged after conjugation.

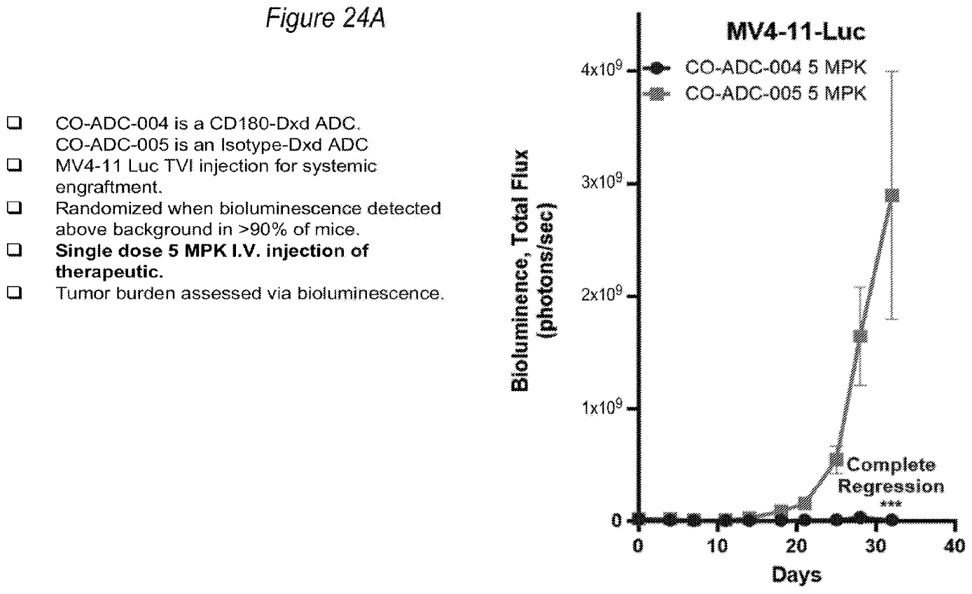

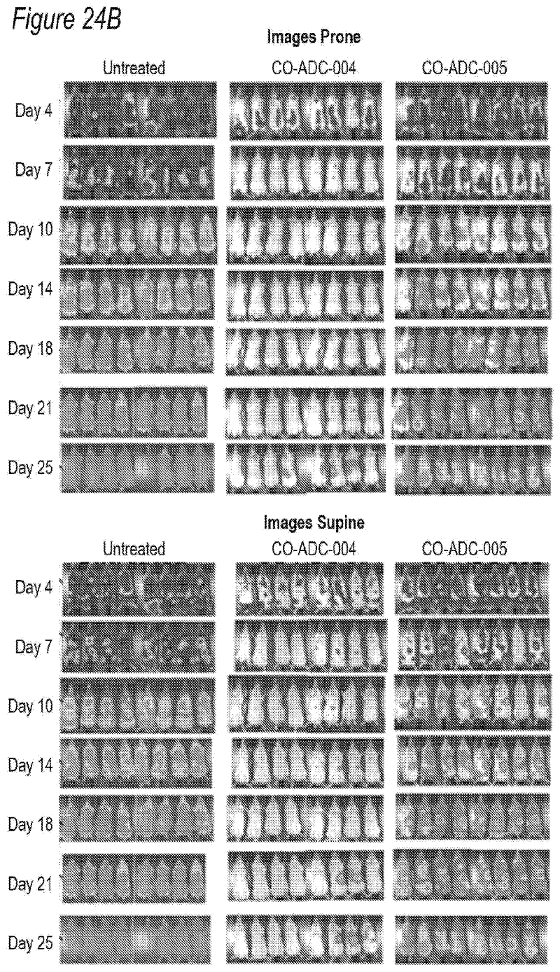

The anti-tumor effectiveness of CO-ADC-004 lead ADC against MV4-11 tumors was tested in vivo. MV4-11 Luc TVI injection (using a Transvalvular Insertion Tool) was used for systemic engraftment of the MV4-11 tumors. Mice were randomized when bioluminescence was detected above background in >90% of mice. A single dose of 5 mg/kg (MPK) was administered to each mouse by I.V. injection of the therapeutic, either CO-ADC-004 or CO-ADC-005. Tumor burden was assessed via bioluminescence. (FIG. 24B) CO-ADC-004 administered at a dose of 5 MPK showed complete regression for over 30 days compared to the Isotype-D×d ADC CO-ADC-005 (FIG. 24A). Accordingly, ADC CO-ADC-004 is potent against MV4-11 tumors in vivo.

### Example 9

### Anti-CD180-Tesirine ADC Effectively Reduced Tumor Burden In Vivo

As noted above, CO-ADC-001 is an anti-CD180 antibody-tesirine ADC. CO-ADC-003 is an isotype-tesirine ADC. The effectiveness of CO-ADC-001 in reducing tumor burden was tested in vivo.

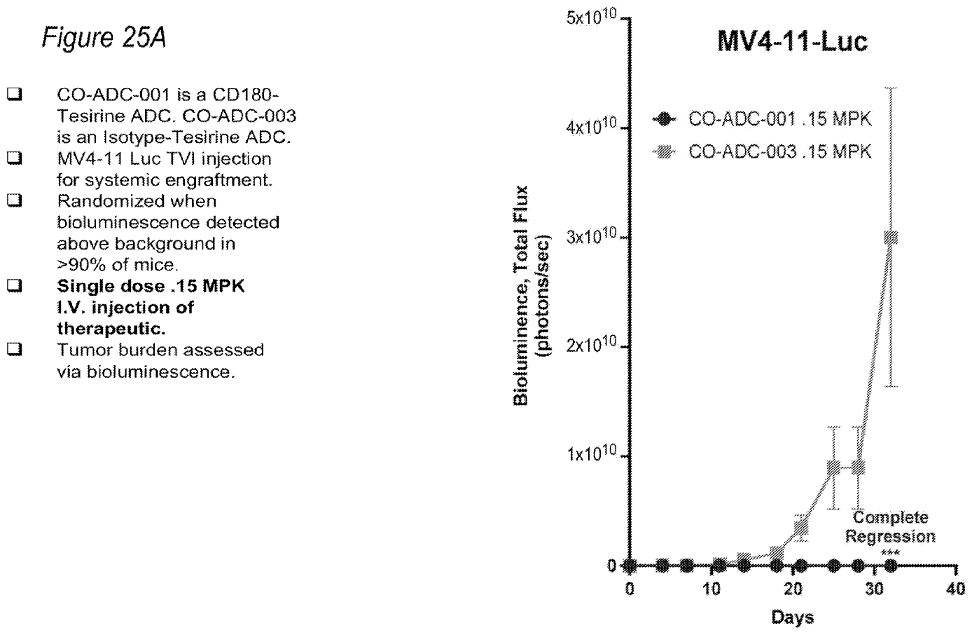

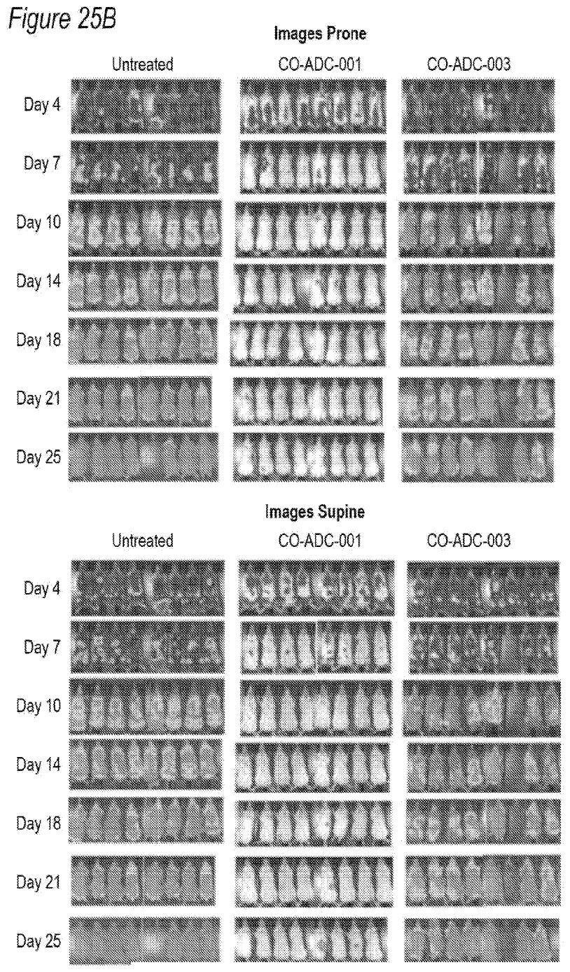

MV4-11 Luc TVI injection was used for systemic engraftment. Mice were randomized when bioluminescence was detected above background in >90% of mice. A single dose of 0.15 mg/kg (MPK) was administered to each mouse by I.V. injection of therapeutic, either CO-ADC-001 ADC or CO-ADC-003. Tumor burden was assessed via bioluminescence. (FIG. 25B) CO-ADC-001 ADC administered at a dose of 0.15 MPK showed complete regression for over 30 days compared to the isotype-tesirine ADC CO-ADC-003 (FIG. 25A).

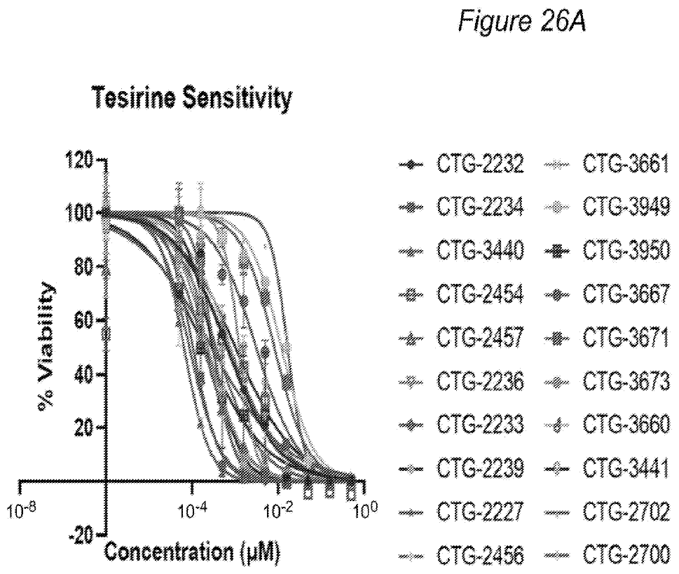

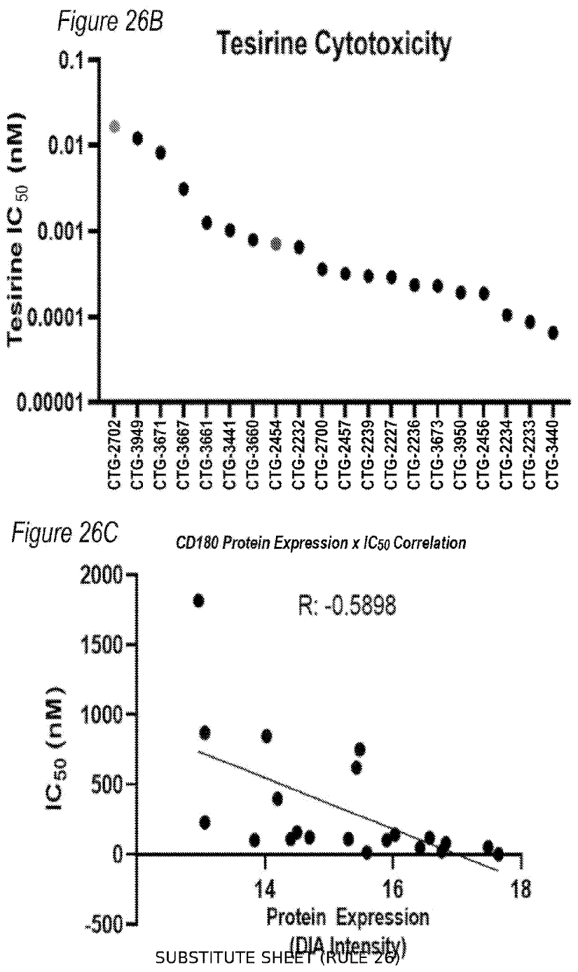

FIGS. 26A-26C show that CD180 protein expression correlates with ADC cytotoxicity. FIGS. 26A-26B show that there is a wide range of sensitivity and cytotoxicity in AML patient samples. FIG. 26C shows CD180 protein expression and IC₅₀ correlation.

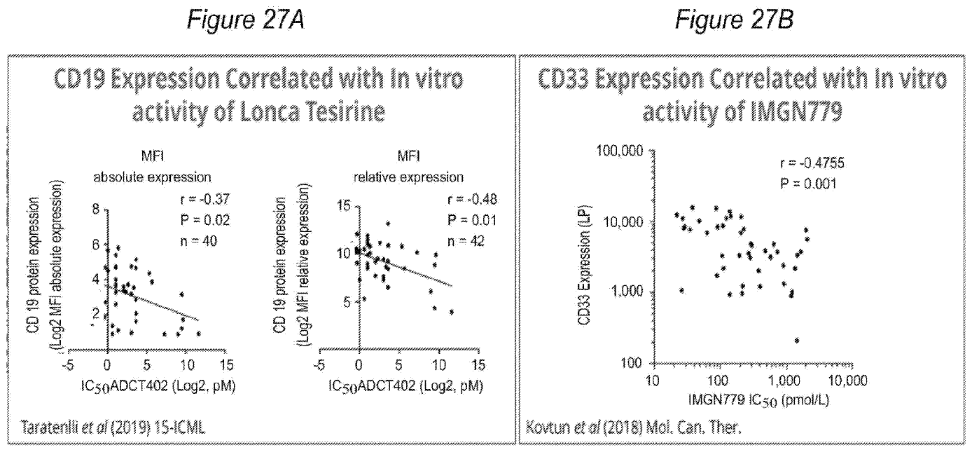

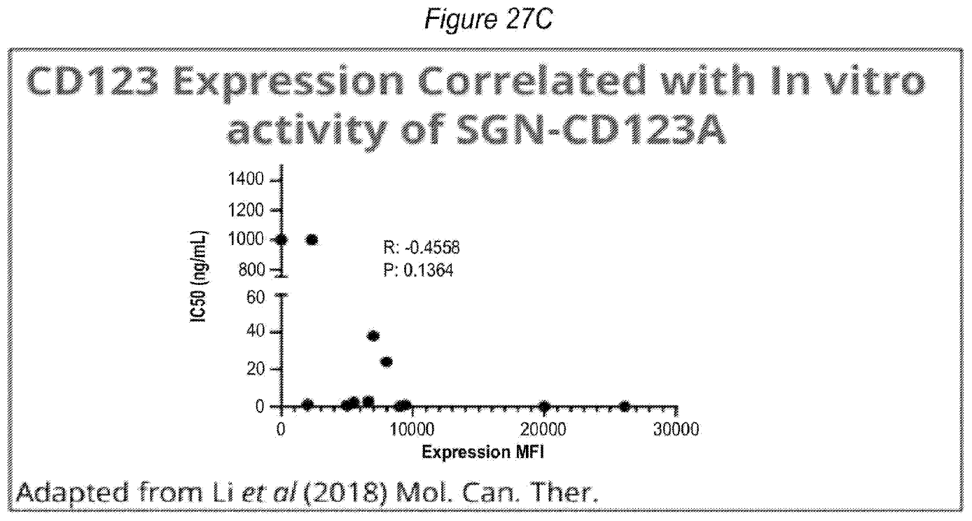

FIGS. 27A-27C show receptor expression correlation with ADC potency in other comparative ADCs. FIG. 27A shows CD19 expression correlated with in vitro activity of Lonca Tesirine. FIG. 27B shows CD33 expression correlated with in vitro activity of IMGN779. FIG. 27 shows CD33 expression correlated with in vitro activity of shows CD123 expression correlated with in vitro activity of SGN-CD123A.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An anti-CD180 antibody-drug conjugate (ADC) comprising a CD180-high expressing tumor-targeting monoclonal antibody or antigen-binding fragment thereof, a cytotoxic drug payload and a linker moiety conjugating the CD180-high expressing tumor-targeting antibody or the antigen-binding fragment thereof to the cytotoxic drug payload, wherein the anti-CD180 antibody or antigen-binding fragment thereof comprises a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each comprise the amino acid sequences:
HCDR1, HCDR2 and HCDR3 of SEQ ID NOs: 1-3 and LCDR1, LCDR2 and LCDR3 of SEQ ID NOs: 4-6;
HCDR1, HCDR2 and HCDR3 of SEQ ID NOs: 1-3 and LCDR1, LCDR2 and LCDR3 of SEQ ID NOs: 4, 5, and 15;
HCDR1, HCDR2 and HCDR3 of SEQ ID NOs: 1, 24, and 25 and LCDR1, LCDR2 and LCDR3 of SEQ ID NOs: 4, 5, and 26;
HCDR1, HCDR2 and HCDR3 of SEQ ID NOs: 1, 35, and 25 and LCDR1, LCDR2 and LCDR3 of SEQ ID NOs: 4, 5, and 26;
HCDR1, HCDR2 and HCDR3 of SEQ ID NOs: 1, 44, and 25 and LCDR1, LCDR2 and LCDR3 of SEQ ID NOs: 4, 5, and 45;
HCDR1, HCDR2 and HCDR3 of SEQ ID NOs: 54-56 and LCDR1, LCDR2 and LCDR3 of SEQ ID NOs: 57-59;
HCDR1, HCDR2 and HCDR3 of SEQ ID NOs: 68-70 and LCDR1, LCDR2 and LCDR3 of SEQ ID NOs: 57, 58, and 71;
HCDR1, HCDR2 and HCDR3 of SEQ ID NOs: 1, 24, and 25 and LCDR1, LCDR2 and LCDR3 of SEQ ID NOs: 80-82;
HCDR1, HCDR2 and HCDR3 of SEQ ID NOs: 1, 35, and 25 and LCDR1, LCDR2 and LCDR3 of SEQ ID NOs: 91-93;
HCDR1, HCDR2 and HCDR3 of SEQ ID NOs: 1, 102, and 25 and LCDR1, LCDR2 and LCDR3 of SEQ ID NOs: 4, 103, and 6;
HCDR1, HCDR2 and HCDR3 of SEQ ID NOs: 1, 24, and 25 and LCDR1, LCDR2 and LCDR3 of SEQ ID NOs: 112, 5, and 113;
HCDR1, HCDR2 and HCDR3 of SEQ ID NOs: 1, 35, and 25 and LCDR1, LCDR2 and LCDR3 of SEQ ID NOs: 4-6;
HCDR1, HCDR2 and HCDR3 of SEQ ID NOs: 1, 44, and 25 and LCDR1, LCDR2 and LCDR3 of SEQ ID NOs: 4, 5, and 130; or
HCDR1, HCDR2 and HCDR3 of SEQ ID NOs: 1, 24, and 25 and LCDR1, LCDR2 and LCDR3 of SEQ ID NOs: 4-6.

2. The ADC of claim 1, wherein the anti-CD180 antibody or antigen-binding fragment thereof comprises a heavy chain variable region and a light chain variable region, said heavy chain variable region and light chain variable region comprise the amino acid sequences of SEQ ID NOs:7-8, SEQ ID NOs:16-17, SEQ ID NOs:27-28, SEQ ID NOs:36-37, SEQ ID NOs:46-47, SEQ ID NOs:60-61, SEQ ID NOs:72-73, SEQ ID NOs:83-84, SEQ ID NOs:94-95, SEQ ID NOs:104-105, SEQ ID NOs:114-115, SEQ ID NOs:122-123, SEQ ID NOs:131-132, or SEQ ID NOs:139-140.

3. The ADC of claim 1, wherein the anti-CD180 antibody or antigen-binding fragment thereof comprises a heavy chain and a light chain, said heavy chain and light chain comprise the amino acid sequences of SEQ ID NOs: 11-12, SEQ ID NOs:20-21, SEQ ID NOs:31-32, SEQ ID NOs:40-41, SEQ ID NOs:50-51, SEQ ID NOs:64-65, SEQ ID NOs:76-77, SEQ ID NOs:87-88, SEQ ID NOs:98-99, SEQ ID NOs:108-109, SEQ ID NOs:118-119, SEQ ID NOs:126-127, SEQ ID NOs:135-136, or SEQ ID NOs:143-144.

4. The ADC of claim 1, wherein the anti-CD180 antibody or antigen-binding fragment thereof comprises an IgG, a Fv, a scFv, a Fab, a F(ab′)₂, a minibody, a diabody, a triabody, a nanobody, a bispecific antibody, a single domain antibody, or a chimeric antigen receptor.

5. The ADC of claim 4, wherein the IgG is IgG1, IgG2, IgG3, or IgG4.

6. The ADC of claim 1, wherein the cytotoxic drug payload comprises a ADC drug-linker conjugate tesirine (SG3249), the tesirine comprising a cytotoxic drug payload, the cytotoxic drug payload comprising pyrrolobenzodiazepine dimer cytotoxic DNA-alkylating agent (SG3199), wherein the tesirine is conjugated to the CD180-high expressing tumor-targeting monoclonal antibody or antigen-binding fragment thereof via a cleavable linker moiety.

7. The ADC of claim 1 comprising an ADC drug-linker conjugate deruxtecan, the deruxetan comprising a cleavable linker, a self-immolative amino methylene spacer, and a cytotoxic drug payload, the cytotoxic drug payload comprising a topoisomerase 1 inhibitor payload, wherein the topoisomerase 1 inhibitor payload is a derivative of exatecan (DX-8951), wherein the deruxetan is conjugated to the CD180-high expressing tumor-targeting monoclonal antibody or antigen-binding fragment thereof via the cleavable linker, wherein the cleavable linker is a glycine-glycine-phenylalanine-glycine tetrapeptide-based linker.

8. The ADC of claim 1, wherein the cleavable linker moiety is a hydrazone linker, a disulphide linker or a peptide linker.

9. The ADC of claim 3, wherein the peptide linker is a dipeptide linker selected from the group consisting of valine-citrulline (Val-Cit), valine-alanine (Val-Ala) and alanine-alanine (Ala-Ala).

10. The ADC of claim 4, wherein the dipeptide linker is joined to the cytotoxic drug payload by a spacer unit, wherein the spacer unit is para-aminobenzyloxycarbonyl (PABC).

11. The ADC of claim 3, wherein the peptide linker is a tripeptide linker, wherein the tripeptide linker is a glutamic acid-valine-citrulline (EVCit) tripeptide linker.

12. The ADC of claim 6, wherein the glutamic acid-valine-citrulline (EVCit) tripeptide linker is joined to a meta-amide para-aminobenzyl carbamate (MA-PABC) group.

13. The ADC of claim 6, wherein the drug-to-antibody ratio (DAR) is 2.1.

14. The ADC of claim 7, wherein the drug-to-antibody ratio (DAR) is 8.

15. A method of treating a cancer in a subject, comprising the step of administering to the subject the ADC of any of claims 1-3 or 6-7.

16. The method of claim 15, wherein the cancer is acute myeloid leukemia (AML), mantle cell lymphoma (MCL), multiple myeloma, follicular lymphoma, B-acute lymphoblastic leukemia, or diffuse large B-cell lymphoma.

17. The method of claim 16, wherein the AML is primary AML or primary MCL.

18. The method of claim 15, wherein the ADC comprises the set of HCDR1, HCDR2 and HCDR3 comprising the amino acids of SEQ ID NOs: 1-3 and the set of corresponding LCDR1, LCDR2 and LCDR3 comprising the amino acid sequences of SEQ ID NOs: 4-6, wherein the cytotoxic drug payload comprises a pyrrolobenzodiazepine dimer cytotoxic alkylating agent tesirine (SG3199) conjugated to the CD180-high expressing tumor-targeting monoclonal antibody or antigen-binding fragment thereof via a cleavable linker moiety, wherein the cleavable linker moiety is a valine-alanine (Val-Ala) peptide linker.

19. The method of claim 18, wherein the Val-Ala peptide linker is cathepsin B-cleavable.

20. The method of claim 18, wherein the DAR is 2.1.

21. The method of claim 18, wherein the cancer is primary AML.

22. The method of claim 18, wherein the cancer is primary MCL.

23. The method of claim 15, wherein the ADC comprises the set of HCDR1, HCDR2 and HCDR3 comprising the amino acids of SEQ ID NOs: 1, 35 and 25 and the set of corresponding LCDR1, LCDR2 and LCDR3 comprising the amino acid sequences of SEQ ID NOs: 4, 5 and 26, wherein the ADC is conjugated to an ADC drug-linker conjugate deruxtecan, the deruxetan comprising a cleavable glycine-glycine-phenylalanine-glycine tetrapeptide-based linker, a self-immolative amino methylene spacer, and a cytotoxic drug payload, the cytotoxic drug payload comprising a topoisomerase 1 inhibitor payload, wherein the topoisomerase 1 inhibitor payload is a derivative of exatecan (DX-8951), wherein the deruxetan is conjugated to the CD180-high expressing tumor-targeting monoclonal antibody or antigen-binding fragment thereof via the cleavable glycine-glycine-phenylalanine-glycine tetrapeptide-based linker.

24. The method of claim 23, wherein the DAR is 8.

25. The method of claim 23, wherein the cancer is primary AML.

26. The method of claim 23, wherein the cancer is primary MCL.

27. A method of modulating CD180-mediated immune responses in a subject, comprising administering to the subject the ADC of any of claims 1-3 or 6-7.

28. The method of claim 27, wherein the CD180-mediated immune responses are antigen presenting cell activities of B cells, antibody secretion, or proliferation of B lymphocytes, monocytes or dendritic cells.

29. The method of claim 27, wherein modulation of CD180-mediated immune responses comprises enhancing said CD180-mediated immune responses or inhibiting said CD180-mediated immune responses.

30. A method of treating a disease in a subject, comprising the step of administering to the subject the ADC of any of claims 1-3 or 6-7.

31. The method of claim 30, wherein the disease is a viral infection, a bacterial infection, an autoimmune disease or an immune disorder.

32. An isolated anti-CD180 binding molecule comprising a set of three heavy chain complementarity determining regions HCDR1, HCDR2 and HCDR3, and a corresponding set of three light chain complementarity determining regions LCDR1, LCDR2 and LCDR3, wherein the set of HCDR1, HCDR2 and HCDR3 and the set of corresponding LCDR1, LCDR2 and LCDR3 each comprise the amino acid sequences:
HCDR1, HCDR2 and HCDR3 of SEQ ID NOs: 1-3 and LCDR1, LCDR2 and LCDR3 of SEQ ID NOs: 4-6;
HCDR1, HCDR2 and HCDR3 of SEQ ID NOs: 1-3 and SEQ ID NOs: 4, 5, and 15;
HCDR1, HCDR2 and HCDR3 of SEQ ID NOs: 1, 24, and 25 and SEQ ID NOs: 4, 5, and 26;
HCDR1, HCDR2 and HCDR3 of SEQ ID NOs: 1, 35, and 25 and SEQ ID NOs: 4, 5, and 26;
HCDR1, HCDR2 and HCDR3 of SEQ ID NOs: 1, 44, and 25 and SEQ ID NOs: 4, 5, and 45;
HCDR1, HCDR2 and HCDR3 of SEQ ID NOs: 54-56 and SEQ ID NOs: 57-59;
HCDR1, HCDR2 and HCDR3 of SEQ ID NOs: 68-70 and SEQ ID NOs: 57, 58, and 71;
HCDR1, HCDR2 and HCDR3 of SEQ ID NOs: 1, 24, and 25 and SEQ ID NOs: 80-82;
HCDR1, HCDR2 and HCDR3 of SEQ ID NOs: 1, 35, and 25 and SEQ ID NOs: 91-93;
HCDR1, HCDR2 and HCDR3 of SEQ ID NOs: 1, 102, and 25 and SEQ ID NOs: 4, 103, and 6;
HCDR1, HCDR2 and HCDR3 of SEQ ID NOs: 1, 24, and 25 and SEQ ID NOs: 112, 5, and 113;
HCDR1, HCDR2 and HCDR3 of SEQ ID NOs: 1, 35, and 25 and SEQ ID NOs: 4-6;
HCDR1, HCDR2 and HCDR3 of SEQ ID NOs: 1, 44, and 25 and SEQ ID NOs: 4, 5, and 130;
HCDR1, HCDR2 and HCDR3 of SEQ ID NOs: 1, 24, and 25 and SEQ ID NOs: 4-6.

33. The anti-CD180 binding molecule of claim 32, wherein the binding molecule comprises a heavy chain variable region and a light chain variable region, said heavy chain variable region and light chain variable region comprise the amino acid sequences of SEQ ID NOs:7-8, SEQ ID NOs:16-17, SEQ ID NOs:27-28, SEQ ID NOs:36-37, SEQ ID NOs:46-47, SEQ ID NOs:60-61, SEQ ID NOs:72-73, SEQ ID NOs:83-84, SEQ ID NOs:94-95, SEQ ID NOs:104-105, SEQ ID NOs:114-115, SEQ ID NOs:122-123, SEQ ID NOs:131-132, or SEQ ID NOs:139-140.

34. The anti-CD180 binding molecule of claim 32, wherein the binding molecule comprises a heavy chain and a light chain, said heavy chain and light chain comprise the amino acid sequences of SEQ ID NOs: 11-12, SEQ ID NOs:20-21, SEQ ID NOs:31-32, SEQ ID NOs:40-41, SEQ ID NOs:50-51, SEQ ID NOs:64-65, SEQ ID NOs:76-77, SEQ ID NOs:87-88, SEQ ID NOs:98-99, SEQ ID NOs:108-109, SEQ ID NOs:118-119, SEQ ID NOs:126-127, SEQ ID NOs:135-136, or SEQ ID NOs:143-144.

35. The anti-CD180 binding molecule of claim 32, wherein the binding molecule comprises an IgG, a Fv, a scFv, a Fab, a F(ab′)₂, a minibody, a diabody, a triabody, a nanobody, a bispecific antibody, a single domain antibody, or a chimeric antigen receptor.

36. The anti-CD180 binding molecule of claim 35, wherein said IgG is IgG1, IgG2, IgG3, or IgG4.

37. An isolated polynucleotide sequence encoding the anti-CD180 binding molecule of any one of claims 32-36.

38. A vector comprising the polynucleotide sequence of claim 37.

39. A host cell comprising the vector of claim 38.

40. A composition comprising the anti-CD180 binding molecule of any one of claims 32-36 and a pharmaceutically acceptable carrier.

41. A composition comprising the ADC of any one of claims 1-3 or 6-7 and a pharmaceutically acceptable carrier.

42. A composition comprising the polynucleotide sequence of claim 37.

43. The composition of claim 42, wherein the polynucleotide sequence comprises an expression vector for expressing the anti-CD180 binding molecule in a cell.

44. A method of modulating CD180-mediated immune responses in a subject, comprising the step of administering to the subject the composition of claim 40 or 41.

45. The method of claim 44, wherein the CD180-mediated immune responses are antigen presenting cell activities of B cells, antibody secretion, or proliferation of B lymphocytes, monocytes or dendritic cells.

46. The method of claim 44, wherein modulation of CD180-mediated immune responses comprises enhancing said CD180-mediated immune responses or inhibiting said CD180-mediated immune responses.

47. A method of treating a disease in a subject, comprising the step of administering to the subject the composition of claim 40 or 41.

48. The method of claim 47, wherein the disease is a viral infection, a bacterial infection, cancer, an autoimmune disease or an immune disorder.

49. The method of claim 47, wherein the disease is a CD180-mediated disease.

50. The method of claim 48, wherein the cancer is acute myeloid leukemia (AML), mantle cell lymphoma (MCL), multiple myeloma, follicular lymphoma, B-acute lymphoblastic leukemia, or diffuse large B-cell lymphoma.

51. A method of treating a CD180 high expressing cancer in a subject, comprising the step of administering to the subject the composition of claim 41.

52. A method of treating a CD180 high expressing cancer in a subject, comprising the step of administering to the subject composition the ADC of claim 6.

53. The method of claim 52, wherein the cancer is acute myeloid leukemia (AML), mantle cell lymphoma (MCL), multiple myeloma, follicular lymphoma, B-acute lymphoblastic leukemia, or diffuse large B-cell lymphoma.

54. The method of claim 52, wherein the cancer is primary AML.

55. The method of claim 52, wherein the cancer is primary MCL.

56. A method of treating a CD180 high expressing cancer in a subject, comprising the step of administering to the subject composition the ADC of claim 7.

57. The method of claim 56, wherein the cancer is acute myeloid leukemia (AML), mantle cell lymphoma (MCL), multiple myeloma, follicular lymphoma, B-acute lymphoblastic leukemia, or diffuse large B-cell lymphoma.

58. The method of claim 56, wherein the cancer is primary AML.

59. The method of claim 56, wherein the cancer is primary MCL.


SEQUENCE LISTING (146 sequences)

SEQ ID NO: 1 - protein, 5 aa, synthetic construct
  REGION 1..5: note = Description of sequence: Name: Ab-001, Chain: HCDR1
DYAMH

SEQ ID NO: 2 - protein, 17 aa, synthetic construct
  REGION 1..17: note = Description of sequence: Name: Ab-001, Chain: HCDR2
YISWNNNNIGYADSVKG

SEQ ID NO: 3 - protein, 11 aa, synthetic construct
  REGION 1..11: note = Description of sequence: Name: Ab-001, Chain: HCDR3
DIAYNTGYFDY

SEQ ID NO: 4 - protein, 12 aa, synthetic construct
  REGION 1..12: note = Description of sequence: Name: Ab-001, Chain: LCDR1
RASQSVSSSYLA

SEQ ID NO: 5 - protein, 7 aa, synthetic construct
  REGION 1..7: note = Description of sequence: Name: Ab-001, Chain: LCDR2
GASSRAT

SEQ ID NO: 6 - protein, 9 aa, synthetic construct
  REGION 1..9: note = Description of sequence: Name: Ab-001, Chain: LCDR3
QQYGSSPLT

SEQ ID NO: 7 - protein, 120 aa, synthetic construct
  REGION 1..120: note = Description of sequence: Name: Ab-001, Chain: VH
EVQLVESGGGLVQPGGSLRLSCAASGFTSLDYAMHWVRQAPGKGLEWVSYISWNNNNIGYADSVKGRFTISRDNAKNSLYLHMNSLRPEDTAFYYCAKDIAYNTGYFDYWGQGTLVTVSS

SEQ ID NO: 8 - protein, 108 aa, synthetic construct
  REGION 1..108: note = Description of sequence: Name: Ab-001, Chain: VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIK

SEQ ID NO: 9 - protein, 360 aa, synthetic construct
  REGION 1..360: note = Description of sequence: Name: Ab-001, Chain: VH
GAAGTGCAGCTGGTGGAGTCTGGGGGAGGCTTGGTACAGCCTGGGGGGTCCCTGAGACTCTCCTGTGCAGCCTCTGGATTCACCTCTCTTGATTATGCCATGCACTGGGTCCGGCAAGCTCCAGGGAAGGGCCTGGAGTGGGTCTCATATATTAGTTGGAATAATAATAACATAGGCTATGCGGACTCTGTGAAGGGCCGATTCACCATCTCCAGAGACAACGCCAAGAACTCCCTGTATCTGCACATGAACAGTCTGAGACCTGAGGACACGGCCTTCTATTACTGTGCAAAAGATATCGCATATAACACCGGCTACTTTGACTACTGGGGCCAGGGAACCCTGGTCACCGTCTCCTCA

SEQ ID NO: 10 - protein, 324 aa, synthetic construct
  REGION 1..324: note = Description of sequence: Name: Ab-001, Chain: VL
GAAATTGTGTTGACGCAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCTCTCACTTTCGGCGGAGGGACCAAGGTGGAAATCAAA

SEQ ID NO: 11 - protein, 449 aa, synthetic construct
  REGION 1..449: note = Description of sequence: Name: Ab-001, Chain: HC
EVQLVESGGGLVQPGGSLRLSCAASGFTSLDYAMHWVRQAPGKGLEWVSYISWNNNNIGYADSVKGRFTISRDNAKNSLYLHMNSLRPEDTAFYYCAKDIAYNTGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 215 aa, synthetic construct
  REGION 1..215: note = Description of sequence: Name: Ab-001, Chain: LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 1347 aa, synthetic construct
  REGION 1..1347: note = Description of sequence: Name: Ab-001, Chain: HC
GAAGTGCAGCTGGTGGAGTCTGGGGGAGGCTTGGTACAGCCTGGGGGGTCCCTGAGACTCTCCTGTGCAGCCTCTGGATTCACCTCTCTTGATTATGCCATGCACTGGGTCCGGCAAGCTCCAGGGAAGGGCCTGGAGTGGGTCTCATATATTAGTTGGAATAATAATAACATAGGCTATGCGGACTCTGTGAAGGGCCGATTCACCATCTCCAGAGACAACGCCAAGAACTCCCTGTATCTGCACATGAACAGTCTGAGACCTGAGGACACGGCCTTCTATTACTGTGCAAAAGATATCGCATATAACACCGGCTACTTTGACTACTGGGGCCAGGGAACCCTGGTCACCGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 14 - protein, 645 aa, synthetic construct
  REGION 1..645: note = Description of sequence: Name: Ab-001, Chain: LC
GAAATTGTGTTGACGCAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCTCTCACTTTCGGCGGAGGGACCAAGGTGGAAATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 15 - protein, 9 aa, synthetic construct
  REGION 1..9: note = Description of sequence: Name: Ab-002, Chain: LCDR3
QQYGSSPFT

SEQ ID NO: 16 - protein, 120 aa, synthetic construct
  REGION 1..120: note = Description of sequence: Name: Ab-002, Chain: VH
EVQLVESGGGLVQPGGSLRLSCAASGFTSVDYAMHWVRQAPGKGLEWVSYISWNNNNIGYADSVKGRFTISRDNAKNSLYLHMSSLRAEDTALYYCAKDIAYNTGYFDYWGQGTLVTVSS

SEQ ID NO: 17 - protein, 108 aa, synthetic construct
  REGION 1..108: note = Description of sequence: Name: Ab-002, Chain: VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVEIK

SEQ ID NO: 18 - protein, 360 aa, synthetic construct
  REGION 1..360: note = Description of sequence: Name: Ab-002, Chain: VH
GAAGTGCAGCTGGTGGAGTCTGGGGGAGGCTTGGTACAGCCTGGGGGGTCCCTGAGACTCTCCTGTGCAGCCTCTGGATTCACCTCTGTTGATTATGCCATGCACTGGGTCCGGCAAGCTCCAGGGAAGGGCCTAGAGTGGGTCTCATATATTAGTTGGAATAATAATAACATAGGCTATGCGGACTCTGTGAAGGGCCGATTCACCATCTCCAGAGACAACGCCAAGAACTCCCTCTATCTGCACATGAGCAGTCTGAGAGCTGAGGACACGGCCTTGTATTATTGTGCAAAAGATATCGCATATAACACCGGGTACTTTGACTACTGGGGCCAGGGAACCCTGGTCACCGTCTCCTCA

SEQ ID NO: 19 - protein, 324 aa, synthetic construct
  REGION 1..324: note = Description of sequence: Name: Ab-002, Chain: VL
GAAATTGTGTTGACGCAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCATTCACTTTCGGCCCTGGGACCAAGGTGGAAATCAAA

SEQ ID NO: 20 - protein, 449 aa, synthetic construct
  REGION 1..449: note = Description of sequence: Name: Ab-002, Chain: HC
EVQLVESGGGLVQPGGSLRLSCAASGFTSVDYAMHWVRQAPGKGLEWVSYISWNNNNIGYADSVKGRFTISRDNAKNSLYLHMSSLRAEDTALYYCAKDIAYNTGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 215 aa, synthetic construct
  REGION 1..215: note = Description of sequence: Name: Ab-002, Chain: LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 1347 aa, synthetic construct
  REGION 1..1347: note = Description of sequence: Name: Ab-002, Chain: HC
GAAGTGCAGCTGGTGGAGTCTGGGGGAGGCTTGGTACAGCCTGGGGGGTCCCTGAGACTCTCCTGTGCAGCCTCTGGATTCACCTCTGTTGATTATGCCATGCACTGGGTCCGGCAAGCTCCAGGGAAGGGCCTAGAGTGGGTCTCATATATTAGTTGGAATAATAATAACATAGGCTATGCGGACTCTGTGAAGGGCCGATTCACCATCTCCAGAGACAACGCCAAGAACTCCCTCTATCTGCACATGAGCAGTCTGAGAGCTGAGGACACGGCCTTGTATTATTGTGCAAAAGATATCGCATATAACACCGGGTACTTTGACTACTGGGGCCAGGGAACCCTGGTCACCGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 23 - protein, 645 aa, synthetic construct
  REGION 1..645: note = Description of sequence: Name: Ab-002, Chain: LC
GAAATTGTGTTGACGCAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCATTCACTTTCGGCCCTGGGACCAAGGTGGAAATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 24 - protein, 17 aa, synthetic construct
  REGION 1..17: note = Description of sequence: Name: Ab-003, Chain: HCDR2
GISWNGGTMGYADSVKG

SEQ ID NO: 25 - protein, 11 aa, synthetic construct
  REGION 1..11: note = Description of sequence: Name: Ab-003, Chain: HCDR3
DIAYSRGYFQH

SEQ ID NO: 26 - protein, 9 aa, synthetic construct
  REGION 1..9: note = Description of sequence: Name: Ab-003, Chain: LCDR3
QQYGSSPRT

SEQ ID NO: 27 - protein, 120 aa, synthetic construct
  REGION 1..120: note = Description of sequence: Name: Ab-003, Chain: VH
EVQLVESGGGLVQPGRSLRLSCTTSGFTFADYAMHWVRQTPGQGLEWVSGISWNGGTMGYADSVKGRFTISRDIAKNSLYLQMNSLRAEDTALYYCAKDIAYSRGYFQHWGQGTLVTVSS

SEQ ID NO: 28 - protein, 108 aa, synthetic construct
  REGION 1..108: note = Description of sequence: Name: Ab-003, Chain: VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 29 - protein, 360 aa, synthetic construct
  REGION 1..360: note = Description of sequence: Name: Ab-003, Chain: VH
GAAGTGCAGCTGGTGGAGTCTGGGGGAGGCTTGGTACAGCCTGGCAGGTCCCTGAGACTCTCCTGTACAACCTCTGGATTCACCTTTGCTGATTATGCCATGCACTGGGTCCGGCAAACTCCAGGGCAGGGCCTGGAGTGGGTCTCAGGTATTAGTTGGAATGGTGGTACCATGGGCTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATCGCATATAGCAGAGGATACTTCCAGCACTGGGGCCAGGGCACCCTGGTCACCGTCTCTTCA

SEQ ID NO: 30 - protein, 324 aa, synthetic construct
  REGION 1..324: note = Description of sequence: Name: Ab-003, Chain: VL
GAAATTGTGTTGACACAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCTCGGACGTTCGGCCAAGGGACCAAGGTGGAAATCAAA

SEQ ID NO: 31 - protein, 449 aa, synthetic construct
  REGION 1..449: note = Description of sequence: Name: Ab-003, Chain: HC
EVQLVESGGGLVQPGRSLRLSCTTSGFTFADYAMHWVRQTPGQGLEWVSGISWNGGTMGYADSVKGRFTISRDIAKNSLYLQMNSLRAEDTALYYCAKDIAYSRGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 215 aa, synthetic construct
  REGION 1..215: note = Description of sequence: Name: Ab-003, Chain: LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 1347 aa, synthetic construct
  REGION 1..1347: note = Description of sequence: Name: Ab-003, Chain: HC
GAAGTGCAGCTGGTGGAGTCTGGGGGAGGCTTGGTACAGCCTGGCAGGTCCCTGAGACTCTCCTGTACAACCTCTGGATTCACCTTTGCTGATTATGCCATGCACTGGGTCCGGCAAACTCCAGGGCAGGGCCTGGAGTGGGTCTCAGGTATTAGTTGGAATGGTGGTACCATGGGCTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATCGCATATAGCAGAGGATACTTCCAGCACTGGGGCCAGGGCACCCTGGTCACCGTCTCTTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 34 - protein, 645 aa, synthetic construct
  REGION 1..645: note = Description of sequence: Name: Ab-003, Chain: LC
GAAATTGTGTTGACACAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCTCGGACGTTCGGCCAAGGGACCAAGGTGGAAATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 35 - protein, 17 aa, synthetic construct
  REGION 1..17: note = Description of sequence: Name: Ab-004, Chain: HCDR2
GISWNGVTIDYADSVKG

SEQ ID NO: 36 - protein, 120 aa, synthetic construct
  REGION 1..120: note = Description of sequence: Name: Ab-004, Chain: VH
EVQLVESGGGLVQPGGSLRLSCTTSGFTFADYAMHWVRQTPGKGLEWVSGISWNGVTIDYADSVKGRFTISRDIAKNSLYLQMNSLRAEDTALYYCAKDIAYSRGYFQHWGQGTLVTVSS

SEQ ID NO: 37 - protein, 108 aa, synthetic construct
  REGION 1..108: note = Description of sequence: Name: Ab-004, Chain: VL
EIVLTQSPATLSVSPGERATLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 38 - protein, 360 aa, synthetic construct
  REGION 1..360: note = Description of sequence: Name: Ab-004, Chain: VH
GAAGTGCAGCTGGTGGAGTCTGGGGGAGGCTTGGTACAGCCTGGGGGGTCCCTGAGACTCTCCTGTACAACCTCTGGATTCACCTTTGCTGATTATGCCATGCACTGGGTCCGGCAAACTCCAGGGAAGGGCCTGGAGTGGGTCTCAGGTATTAGTTGGAATGGTGTTACCATTGACTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATAGCATATAGCAGAGGATACTTCCAGCACTGGGGCCAGGGCACCCTGGTCACCGTCTCCTCA

SEQ ID NO: 39 - protein, 324 aa, synthetic construct
  REGION 1..324: note = Description of sequence: Name: Ab-004, Chain: VL
GAAATTGTGTTGACGCAGTCTCCAGCCACCCTGTCTGTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCTCGGACGTTCGGCCAAGGGACCAAGGTGGAGATCAAA

SEQ ID NO: 40 - protein, 449 aa, synthetic construct
  REGION 1..449: note = Description of sequence: Name: Ab-004, Chain: HC
EVQLVESGGGLVQPGGSLRLSCTTSGFTFADYAMHWVRQTPGKGLEWVSGISWNGVTIDYADSVKGRFTISRDIAKNSLYLQMNSLRAEDTALYYCAKDIAYSRGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 215 aa, synthetic construct
  REGION 1..215: note = Description of sequence: Name: Ab-004, Chain: LC
EIVLTQSPATLSVSPGERATLSCRASQSVSSSYLAWYQQKPGQAPRLLIYGASSRATGIPDRFSGSGSGTDFTLTISRLEPEDFAVYYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 1347 aa, synthetic construct
  REGION 1..1347: note = Description of sequence: Name: Ab-004, Chain: HC
GAAGTGCAGCTGGTGGAGTCTGGGGGAGGCTTGGTACAGCCTGGGGGGTCCCTGAGACTCTCCTGTACAACCTCTGGATTCACCTTTGCTGATTATGCCATGCACTGGGTCCGGCAAACTCCAGGGAAGGGCCTGGAGTGGGTCTCAGGTATTAGTTGGAATGGTGTTACCATTGACTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATAGCATATAGCAGAGGATACTTCCAGCACTGGGGCCAGGGCACCCTGGTCACCGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 43 - protein, 645 aa, synthetic construct
  REGION 1..645: note = Description of sequence: Name: Ab-004, Chain: LC
GAAATTGTGTTGACGCAGTCTCCAGCCACCCTGTCTGTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCTCGGACGTTCGGCCAAGGGACCAAGGTGGAGATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 44 - protein, 17 aa, synthetic construct
  REGION 1..17: note = Description of sequence: Name: Ab-005, Chain: HCDR2
GISWNGGTIGYADSVKG

SEQ ID NO: 45 - protein, 9 aa, synthetic construct
  REGION 1..9: note = Description of sequence: Name: Ab-005, Chain: LCDR3
QQYGSSPYT

SEQ ID NO: 46 - protein, 120 aa, synthetic construct
  REGION 1..120: note = Description of sequence: Name: Ab-005, Chain: VH
EVQLVESGGGLVQPGGSLRLSCTTSGFTFADYAMHWVRQTPGKGLEWVSGISWNGGTIGYADSVKGRFTISRDIAKNSLYLQMNSLRAEDTALYYCAKDIAYSRGYFQHWGQGTLVTVSS

SEQ ID NO: 47 - protein, 108 aa, synthetic construct
  REGION 1..108: note = Description of sequence: Name: Ab-005, Chain: VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKVDIK

SEQ ID NO: 48 - protein, 360 aa, synthetic construct
  REGION 1..360: note = Description of sequence: Name: Ab-005, Chain: VH
GAAGTGCAGCTGGTGGAGTCTGGGGGAGGCTTGGTACAGCCTGGCGGGTCCCTGAGACTCTCCTGTACAACCTCTGGATTCACCTTTGCTGATTATGCCATGCACTGGGTCCGGCAAACTCCAGGGAAGGGCCTGGAGTGGGTCTCAGGTATTAGTTGGAATGGTGGTACCATAGGCTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATAGCATATAGCAGAGGGTACTTTCAGCACTGGGGCCAGGGCACCCTGGTCACCGTCTCCTCA

SEQ ID NO: 49 - protein, 324 aa, synthetic construct
  REGION 1..324: note = Description of sequence: Name: Ab-005, Chain: VL
GAAATTGTGTTGACACAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCGTACACTTTTGGCCAGGGGACCAAAGTGGATATCAAA

SEQ ID NO: 50 - protein, 449 aa, synthetic construct
  REGION 1..449: note = Description of sequence: Name: Ab-005, Chain: HC
EVQLVESGGGLVQPGGSLRLSCTTSGFTFADYAMHWVRQTPGKGLEWVSGISWNGGTIGYADSVKGRFTISRDIAKNSLYLQMNSLRAEDTALYYCAKDIAYSRGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 215 aa, synthetic construct
  REGION 1..215: note = Description of sequence: Name: Ab-005, Chain: LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 1347 aa, synthetic construct
  REGION 1..1347: note = Description of sequence: Name: Ab-005, Chain: HC
GAAGTGCAGCTGGTGGAGTCTGGGGGAGGCTTGGTACAGCCTGGCGGGTCCCTGAGACTCTCCTGTACAACCTCTGGATTCACCTTTGCTGATTATGCCATGCACTGGGTCCGGCAAACTCCAGGGAAGGGCCTGGAGTGGGTCTCAGGTATTAGTTGGAATGGTGGTACCATAGGCTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATAGCATATAGCAGAGGGTACTTTCAGCACTGGGGCCAGGGCACCCTGGTCACCGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 53 - protein, 645 aa, synthetic construct
  REGION 1..645: note = Description of sequence: Name: Ab-005, Chain: LC
GAAATTGTGTTGACACAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCGTACACTTTTGGCCAGGGGACCAAAGTGGATATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 54 - protein, 5 aa, synthetic construct
  REGION 1..5: note = Description of sequence: Name: Ab-006, Chain: HCDR1
DYDMT

SEQ ID NO: 55 - protein, 17 aa, synthetic construct
  REGION 1..17: note = Description of sequence: Name: Ab-006, Chain: HCDR2
GINWNGGNTDYADSVKG

SEQ ID NO: 56 - protein, 20 aa, synthetic construct
  REGION 1..20: note = Description of sequence: Name: Ab-006, Chain: HCDR3
KGRDIVVVKGFYYHYYYMDV

SEQ ID NO: 57 - protein, 16 aa, synthetic construct
  REGION 1..16: note = Description of sequence: Name: Ab-006, Chain: LCDR1
RSSQSLLHSNGYNYLD

SEQ ID NO: 58 - protein, 7 aa, synthetic construct
  REGION 1..7: note = Description of sequence: Name: Ab-006, Chain: LCDR2
LGSNRAS

SEQ ID NO: 59 - protein, 9 aa, synthetic construct
  REGION 1..9: note = Description of sequence: Name: Ab-006, Chain: LCDR3
MQALQTPLT

SEQ ID NO: 60 - protein, 129 aa, synthetic construct
  REGION 1..129: note = Description of sequence: Name: Ab-006, Chain: VH
EVQLVESGGGLVKPGGSLRLSCTASGFTFDDYDMTWVRQAPGKGLEWVSGINWNGGNTDYADSVKGRFTISRDNTKNSLYLQMNSLRAEDTALYYCARKGRDIVVVKGFYYHYYYMDVWGKGITVTVSS

SEQ ID NO: 61 - protein, 112 aa, synthetic construct
  REGION 1..112: note = Description of sequence: Name: Ab-006, Chain: VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVDIK

SEQ ID NO: 62 - protein, 387 aa, synthetic construct
  REGION 1..387: note = Description of sequence: Name: Ab-006, Chain: VH
GAGGTGCAGCTGGTGGAGTCTGGGGGAGGCCTGGTCAAGCCTGGGGGGTCCCTGAGACTCTCCTGTACAGCCTCTGGATTCACCTTTGATGATTATGACATGACCTGGGTCCGCCAAGCTCCAGGGAAGGGGCTGGAGTGGGTCTCTGGTATTAATTGGAATGGTGGTAACACAGATTATGCAGACTCTGTGAAGGGCCGATTCACCATCTCCAGAGACAACACCAAGAACTCCCTGTATCTGCAGATGAACAGTCTGAGAGCCGAGGACACGGCCTTGTATTACTGTGCGAGAAAAGGGAGGGATATTGTAGTAGTAAAAGGATTTTACTACCACTACTACTACATGGACGTCTGGGGCAAAGGGATCACGGTCACTGTCTCCTCA

SEQ ID NO: 63 - protein, 336 aa, synthetic construct
  REGION 1..336: note = Description of sequence: Name: Ab-006, Chain: VL
GATATTGTGATGACCCAGTCTCCACTCTCCCTGCCCGTCACCCCTGGAGAGCCGGCCTCCATCTCCTGCAGGTCTAGTCAGAGCCTCCTGCATAGTAATGGATACAACTATTTGGATTGGTACCTGCAGAAGCCAGGGCAGTCTCCACAGCTCCTGATCTATTTGGGTTCTAATCGGGCCTCCGGGGTCCCTGACAGGTTCAGTGGCAGTGGATCAGGCACAGATTTTACACTGAAAATCAGCAGAGTGGAGGCTGAGGATGTTGGGGTTTATTACTGCATGCAAGCTCTACAAACTCCGCTCACTTTCGGCGGAGGGACCAAAGTGGATATCAAA

SEQ ID NO: 64 - protein, 458 aa, synthetic construct
  REGION 1..458: note = Description of sequence: Name: Ab-006, Chain: HC
EVQLVESGGGLVKPGGSLRLSCTASGFTFDDYDMTWVRQAPGKGLEWVSGINWNGGNTDYADSVKGRFTISRDNTKNSLYLQMNSLRAEDTALYYCARKGRDIVVVKGFYYHYYYMDVWGKGITVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 219 aa, synthetic construct
  REGION 1..219: note = Description of sequence: Name: Ab-006, Chain: LC
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 1374 aa, synthetic construct
  REGION 1..1374: note = Description of sequence: Name: Ab-006, Chain: HC
GAGGTGCAGCTGGTGGAGTCTGGGGGAGGCCTGGTCAAGCCTGGGGGGTCCCTGAGACTCTCCTGTACAGCCTCTGGATTCACCTTTGATGATTATGACATGACCTGGGTCCGCCAAGCTCCAGGGAAGGGGCTGGAGTGGGTCTCTGGTATTAATTGGAATGGTGGTAACACAGATTATGCAGACTCTGTGAAGGGCCGATTCACCATCTCCAGAGACAACACCAAGAACTCCCTGTATCTGCAGATGAACAGTCTGAGAGCCGAGGACACGGCCTTGTATTACTGTGCGAGAAAAGGGAGGGATATTGTAGTAGTAAAAGGATTTTACTACCACTACTACTACATGGACGTCTGGGGCAAAGGGATCACGGTCACTGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 67 - protein, 657 aa, synthetic construct
  REGION 1..657: note = Description of sequence: Name: Ab-006, Chain: LC
GATATTGTGATGACCCAGTCTCCACTCTCCCTGCCCGTCACCCCTGGAGAGCCGGCCTCCATCTCCTGCAGGTCTAGTCAGAGCCTCCTGCATAGTAATGGATACAACTATTTGGATTGGTACCTGCAGAAGCCAGGGCAGTCTCCACAGCTCCTGATCTATTTGGGTTCTAATCGGGCCTCCGGGGTCCCTGACAGGTTCAGTGGCAGTGGATCAGGCACAGATTTTACACTGAAAATCAGCAGAGTGGAGGCTGAGGATGTTGGGGTTTATTACTGCATGCAAGCTCTACAAACTCCGCTCACTTTCGGCGGAGGGACCAAAGTGGATATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 68 - protein, 5 aa, synthetic construct
  REGION 1..5: note = Description of sequence: Name: Ab-007, Chain: HCDR1
DYAMS

SEQ ID NO: 69 - protein, 17 aa, synthetic construct
  REGION 1..17: note = Description of sequence: Name: Ab-007, Chain: HCDR2
DINWNGGTTSYADSVKG

SEQ ID NO: 70 - protein, 20 aa, synthetic construct
  REGION 1..20: note = Description of sequence: Name: Ab-007, Chain: HCDR3
KGRDTVVVKGYYYFSYYMDV

SEQ ID NO: 71 - protein, 9 aa, synthetic construct
  REGION 1..9: note = Description of sequence: Name: Ab-007, Chain: LCDR3
MQALQTPWT

SEQ ID NO: 72 - protein, 129 aa, synthetic construct
  REGION 1..129: note = Description of sequence: Name: Ab-007, Chain: VH
EVQLVESGGGVVRPGGSLRLSCAASGFTFDDYAMSWVRQAPGKGLEWVSDINWNGGTTSYADSVKGRFTISRDNAKNSLFLQMNSLRAEDTALYYCARKGRDTVVVKGYYYFSYYMDVWGKGSTVTVSS

SEQ ID NO: 73 - protein, 112 aa, synthetic construct
  REGION 1..112: note = Description of sequence: Name: Ab-007, Chain: VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKLEIK

SEQ ID NO: 74 - protein, 387 aa, synthetic construct
  REGION 1..387: note = Description of sequence: Name: Ab-007, Chain: VH
GAGGTGCAGCTGGTGGAGTCTGGGGGAGGTGTGGTTCGGCCTGGGGGGTCCCTGAGACTCTCCTGTGCAGCCTCTGGATTCACCTTTGATGATTATGCCATGAGTTGGGTCCGCCAAGCTCCAGGGAAGGGGCTGGAGTGGGTCTCTGATATTAATTGGAATGGTGGTACCACAAGTTATGCAGACTCTGTGAAGGGCCGATTCACCATCTCCAGAGACAACGCCAAGAACTCCCTCTTTCTACAAATGAACAGTCTGAGAGCCGAGGACACGGCCTTGTATTACTGTGCGAGAAAAGGGAGGGATACTGTAGTAGTAAAAGGGTATTATTATTTTTCCTACTACATGGACGTCTGGGGCAAAGGGAGTACGGTCACCGTCTCCTCA

SEQ ID NO: 75 - protein, 336 aa, synthetic construct
  REGION 1..336: note = Description of sequence: Name: Ab-007, Chain: VL
GATATTGTGATGACTCAGTCTCCACTCTCCCTGCCCGTCACCCCTGGAGAGCCGGCCTCCATCTCCTGCAGGTCTAGTCAGAGCCTCCTGCATAGTAATGGATACAACTATTTGGATTGGTACCTGCAGAAGCCAGGGCAGTCTCCACAGCTCCTGATCTATTTGGGTTCTAATCGGGCCTCCGGGGTCCCTGACAGGTTCAGTGGCAGTGGATCAGGCACAGATTTTACACTGAAAATCAGCAGAGTGGAGGCTGAGGATGTTGGGGTTTATTACTGCATGCAAGCTCTACAAACTCCGTGGACGTTCGGCCAAGGGACCAAGCTGGAAATCAAA

SEQ ID NO: 76 - protein, 458 aa, synthetic construct
  REGION 1..458: note = Description of sequence: Name: Ab-007, Chain: HC
EVQLVESGGGVVRPGGSLRLSCAASGFTFDDYAMSWVRQAPGKGLEWVSDINWNGGTTSYADSVKGRFTISRDNAKNSLFLQMNSLRAEDTALYYCARKGRDTVVVKGYYYFSYYMDVWGKGSTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 219 aa, synthetic construct
  REGION 1..219: note = Description of sequence: Name: Ab-007, Chain: LC
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 1374 aa, synthetic construct
  REGION 1..1374: note = Description of sequence: Name: Ab-007, Chain: HC
GAGGTGCAGCTGGTGGAGTCTGGGGGAGGTGTGGTTCGGCCTGGGGGGTCCCTGAGACTCTCCTGTGCAGCCTCTGGATTCACCTTTGATGATTATGCCATGAGTTGGGTCCGCCAAGCTCCAGGGAAGGGGCTGGAGTGGGTCTCTGATATTAATTGGAATGGTGGTACCACAAGTTATGCAGACTCTGTGAAGGGCCGATTCACCATCTCCAGAGACAACGCCAAGAACTCCCTCTTTCTACAAATGAACAGTCTGAGAGCCGAGGACACGGCCTTGTATTACTGTGCGAGAAAAGGGAGGGATACTGTAGTAGTAAAAGGGTATTATTATTTTTCCTACTACATGGACGTCTGGGGCAAAGGGAGTACGGTCACCGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 79 - protein, 657 aa, synthetic construct
  REGION 1..657: note = Description of sequence: Name: Ab-007, Chain: LC
GATATTGTGATGACTCAGTCTCCACTCTCCCTGCCCGTCACCCCTGGAGAGCCGGCCTCCATCTCCTGCAGGTCTAGTCAGAGCCTCCTGCATAGTAATGGATACAACTATTTGGATTGGTACCTGCAGAAGCCAGGGCAGTCTCCACAGCTCCTGATCTATTTGGGTTCTAATCGGGCCTCCGGGGTCCCTGACAGGTTCAGTGGCAGTGGATCAGGCACAGATTTTACACTGAAAATCAGCAGAGTGGAGGCTGAGGATGTTGGGGTTTATTACTGCATGCAAGCTCTACAAACTCCGTGGACGTTCGGCCAAGGGACCAAGCTGGAAATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 80 - protein, 11 aa, synthetic construct
  REGION 1..11: note = Description of sequence: Name: Ab-008, Chain: LCDR1
RASQGIRNDLG

SEQ ID NO: 81 - protein, 7 aa, synthetic construct
  REGION 1..7: note = Description of sequence: Name: Ab-008, Chain: LCDR2
AASTLQS

SEQ ID NO: 82 - protein, 9 aa, synthetic construct
  REGION 1..9: note = Description of sequence: Name: Ab-008, Chain: LCDR3
QKYNSAPFT

SEQ ID NO: 83 - protein, 120 aa, synthetic construct; identical to SEQ ID NO: 27
  REGION 1..120: note = Description of sequence: Name: Ab-008, Chain: VH

SEQ ID NO: 84 - protein, 107 aa, synthetic construct
  REGION 1..107: note = Description of sequence: Name: Ab-008, Chain: VL
DIQMTQSPSSLSASVGDRVTITCRASQGIRNDLGWYQQKPGKAPKLLIYAASTLQSGVPSRFSGSGSGTDFTLTISSLQPEDVATYYCQKYNSAPFTFGPGTKVDIK

SEQ ID NO: 85 - protein, 360 aa, synthetic construct
  REGION 1..360: note = Description of sequence: Name: Ab-008, Chain: VH
GAAGTGCAGCTGGTGGAGTCTGGGGGAGGCTTGGTACAGCCTGGCAGGTCCCTGAGACTCTCCTGTACAACCTCTGGATTCACCTTTGCTGATTATGCCATGCACTGGGTCCGGCAAACTCCAGGGCAGGGCCTGGAGTGGGTCTCAGGTATTAGTTGGAATGGTGGTACCATGGGCTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATCGCATATAGCAGAGGATACTTCCAGCACTGGGGCCAGGGCACCCTGGTCACCGTCTCCTCA

SEQ ID NO: 86 - protein, 321 aa, synthetic construct
  REGION 1..321: note = Description of sequence: Name: Ab-008, Chain: VL
GACATCCAGATGACCCAGTCTCCATCCTCCCTGTCTGCATCTGTAGGAGACAGAGTCACCATCACTTGCCGGGCAAGTCAGGGCATTAGAAATGATTTAGGCTGGTATCAGCAGAAACCAGGGAAAGCCCCTAAGCTCCTGATCTATGCTGCATCCACTTTGCAATCAGGGGTCCCATCTCGGTTCAGTGGCAGTGGATCTGGGACAGATTTCACTCTCACCATCAGCAGCCTGCAGCCTGAAGATGTTGCAACTTATTACTGTCAAAAGTATAACAGTGCCCCATTCACTTTCGGCCCTGGGACCAAAGTGGATATCAAA

SEQ ID NO: 87 - protein, 449 aa, synthetic construct; identical to SEQ ID NO: 31
  REGION 1..449: note = Description of sequence: Name: Ab-008, Chain: HC

SEQ ID NO: 88 - protein, 214 aa, synthetic construct
  REGION 1..214: note = Description of sequence: Name: Ab-008, Chain: LC
DIQMTQSPSSLSASVGDRVTITCRASQGIRNDLGWYQQKPGKAPKLLIYAASTLQSGVPSRFSGSGSGTDFTLTISSLQPEDVATYYCQKYNSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 1347 aa, synthetic construct
  REGION 1..1347: note = Description of sequence: Name: Ab-008, Chain: HC
GAAGTGCAGCTGGTGGAGTCTGGGGGAGGCTTGGTACAGCCTGGCAGGTCCCTGAGACTCTCCTGTACAACCTCTGGATTCACCTTTGCTGATTATGCCATGCACTGGGTCCGGCAAACTCCAGGGCAGGGCCTGGAGTGGGTCTCAGGTATTAGTTGGAATGGTGGTACCATGGGCTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATCGCATATAGCAGAGGATACTTCCAGCACTGGGGCCAGGGCACCCTGGTCACCGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 90 - protein, 642 aa, synthetic construct
  REGION 1..642: note = Description of sequence: Name: Ab-008, Chain: LC
GACATCCAGATGACCCAGTCTCCATCCTCCCTGTCTGCATCTGTAGGAGACAGAGTCACCATCACTTGCCGGGCAAGTCAGGGCATTAGAAATGATTTAGGCTGGTATCAGCAGAAACCAGGGAAAGCCCCTAAGCTCCTGATCTATGCTGCATCCACTTTGCAATCAGGGGTCCCATCTCGGTTCAGTGGCAGTGGATCTGGGACAGATTTCACTCTCACCATCAGCAGCCTGCAGCCTGAAGATGTTGCAACTTATTACTGTCAAAAGTATAACAGTGCCCCATTCACTTTCGGCCCTGGGACCAAAGTGGATATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 91 - protein, 17 aa, synthetic construct
  REGION 1..17: note = Description of sequence: Name: Ab-009, Chain: LCDR1
KSSQNVLYSSNNENHLA

SEQ ID NO: 92 - protein, 7 aa, synthetic construct
  REGION 1..7: note = Description of sequence: Name: Ab-009, Chain: LCDR2
WASTRES

SEQ ID NO: 93 - protein, 9 aa, synthetic construct
  REGION 1..9: note = Description of sequence: Name: Ab-009, Chain: LCDR3
QQYYSTPYT

SEQ ID NO: 94 - protein, 120 aa, synthetic construct
  REGION 1..120: note = Description of sequence: Name: Ab-009, Chain: VH
EVQLVESGGGLVQPGRSLRLSCTTSGFTFADYAMHWVRQTPGKGLEWVSGISWNGVTIDYADSVKGRFTISRDIAKNSLYLQMNSLRAEDTALYYCAKDIAYSRGYFQHWGQGTLVTVSS

SEQ ID NO: 95 - protein, 113 aa, synthetic construct
  REGION 1..113: note = Description of sequence: Name: Ab-009, Chain: VL
DIVMTQSPDSLAVSLGERATVNCKSSQNVLYSSNNENHLAWYQQKPGQPPKLLIYWASTRESGVPDRFSGSGSGTDFTLTISSLQAEDVAIYYCQQYYSTPYTFGQGTKVDIK

SEQ ID NO: 96 - protein, 360 aa, synthetic construct
  REGION 1..360: note = Description of sequence: Name: Ab-009, Chain: VH
GAAGTGCAGCTGGTGGAGTCTGGGGGAGGCTTGGTACAGCCTGGCAGGTCCCTGAGACTCTCCTGTACAACCTCTGGATTCACCTTTGCTGATTATGCCATGCACTGGGTCCGGCAAACTCCAGGGAAGGGCCTGGAGTGGGTCTCAGGTATTAGTTGGAATGGTGTTACCATTGACTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATAGCATATAGCAGAGGATACTTCCAGCACTGGGGCCAGGGCACCCTGGTCACTGTCTCCTCA

SEQ ID NO: 97 - protein, 339 aa, synthetic construct
  REGION 1..339: note = Description of sequence: Name: Ab-009, Chain: VL
GACATCGTGATGACCCAGTCTCCAGACTCCCTGGCTGTGTCTCTGGGCGAGAGGGCCACCGTCAACTGCAAGTCCAGCCAGAATGTTTTATACAGTTCCAACAATGAGAATCACTTAGCTTGGTACCAGCAGAAACCAGGACAGCCTCCTAAGCTGCTCATTTACTGGGCATCTACCCGGGAATCCGGGGTCCCTGACCGATTCAGTGGCAGCGGGTCTGGGACAGATTTCACTCTCACCATCAGCAGCCTGCAGGCTGAAGATGTGGCAATTTATTACTGTCAACAATATTATAGTACTCCGTACACTTTTGGCCAGGGGACCAAAGTGGATATCAAA

SEQ ID NO: 98 - protein, 449 aa, synthetic construct
  REGION 1..449: note = Description of sequence: Name: Ab-009, Chain: HC
EVQLVESGGGLVQPGRSLRLSCTTSGFTFADYAMHWVRQTPGKGLEWVSGISWNGVTIDYADSVKGRFTISRDIAKNSLYLQMNSLRAEDTALYYCAKDIAYSRGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 220 aa, synthetic construct
  REGION 1..220: note = Description of sequence: Name: Ab-009, Chain: LC
DIVMTQSPDSLAVSLGERATVNCKSSQNVLYSSNNENHLAWYQQKPGQPPKLLIYWASTRESGVPDRFSGSGSGTDFTLTISSLQAEDVAIYYCQQYYSTPYTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 1347 aa, synthetic construct
  REGION 1..1347: note = Description of sequence: Name: Ab-009, Chain: HC
GAAGTGCAGCTGGTGGAGTCTGGGGGAGGCTTGGTACAGCCTGGCAGGTCCCTGAGACTCTCCTGTACAACCTCTGGATTCACCTTTGCTGATTATGCCATGCACTGGGTCCGGCAAACTCCAGGGAAGGGCCTGGAGTGGGTCTCAGGTATTAGTTGGAATGGTGTTACCATTGACTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATAGCATATAGCAGAGGATACTTCCAGCACTGGGGCCAGGGCACCCTGGTCACTGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 101 - protein, 660 aa, synthetic construct
  REGION 1..660: note = Description of sequence: Name: Ab-009, Chain: LC
GACATCGTGATGACCCAGTCTCCAGACTCCCTGGCTGTGTCTCTGGGCGAGAGGGCCACCGTCAACTGCAAGTCCAGCCAGAATGTTTTATACAGTTCCAACAATGAGAATCACTTAGCTTGGTACCAGCAGAAACCAGGACAGCCTCCTAAGCTGCTCATTTACTGGGCATCTACCCGGGAATCCGGGGTCCCTGACCGATTCAGTGGCAGCGGGTCTGGGACAGATTTCACTCTCACCATCAGCAGCCTGCAGGCTGAAGATGTGGCAATTTATTACTGTCAACAATATTATAGTACTCCGTACACTTTTGGCCAGGGGACCAAAGTGGATATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 102 - protein, 17 aa, synthetic construct
  REGION 1..17: note = Description of sequence: Name: Ab-010, Chain: HCDR2
GISWNGGTFDYADSVKG

SEQ ID NO: 103 - protein, 7 aa, synthetic construct
  REGION 1..7: note = Description of sequence: Name: Ab-010, Chain: LCDR2
GASTRAT

SEQ ID NO: 104 - protein, 120 aa, synthetic construct
  REGION 1..120: note = Description of sequence: Name: Ab-010, Chain: VH
EVQLVESGGGLVQPGGSLRLSCTSSGFTFADYAMHWVRQTPGKGLEWVSGISWNGGTFDYADSVKGRFTISRDIAKNSLYLQMNSLRVEDTALYYCAKDIAYSRGYFQHWGQGTLVTVSS

SEQ ID NO: 105 - protein, 108 aa, synthetic construct
  REGION 1..108: note = Description of sequence: Name: Ab-010, Chain: VL
EIVLTQSPGTLSLSPGERATLSCRASQSVSSSYLAWYQQKPGQAPRLLIYGASTRATGIPDRFSGSGSGTDFTLTISRLEPEDFAVYYCQQYGSSPLTFGGGTKVEIK

SEQ ID NO: 106 - protein, 360 aa, synthetic construct
  REGION 1..360: note = Description of sequence: Name: Ab-010, Chain: VH
GAAGTGCAGCTGGTGGAGTCTGGGGGAGGCTTGGTACAGCCTGGGGGGTCCCTGAGACTCTCCTGTACATCCTCTGGATTCACCTTTGCTGATTATGCCATGCACTGGGTCCGGCAAACTCCAGGGAAGGGCCTGGAGTGGGTCTCAGGTATTAGTTGGAATGGTGGTACCTTTGACTATGCGGACTCTGTGAAGGGCCGATTCACCATCTCCAGAGACATCGCCAAGAACTCCCTGTATCTGCAAATGAACAGTCTGAGAGTTGAGGACACGGCCTTATATTATTGTGCAAAGGATATAGCATATAGCAGAGGATACTTCCAGCACTGGGGCCAGGGCACCCTGGTCACCGTCTCCTCA

SEQ ID NO: 107 - protein, 324 aa, synthetic construct
  REGION 1..324: note = Description of sequence: Name: Ab-010, Chain: VL
GAAATTGTGTTGACGCAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCACCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCTCTCACTTTCGGCGGAGGGACCAAGGTGGAGATCAAA

SEQ ID NO: 108 - protein, 449 aa, synthetic construct
  REGION 1..449: note = Description of sequence: Name: Ab-010, Chain: HC
EVQLVESGGGLVQPGGSLRLSCTSSGFTFADYAMHWVRQTPGKGLEWVSGISWNGGTFDYADSVKGRFTISRDIAKNSLYLQMNSLRVEDTALYYCAKDIAYSRGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 215 aa, synthetic construct
  REGION 1..215: note = Description of sequence: Name: Ab-010, Chain: LC
EIVLTQSPGTLSLSPGERATLSCRASQSVSSSYLAWYQQKPGQAPRLLIYGASTRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 1347 aa, synthetic construct
  REGION 1..1347: note = Description of sequence: Name: Ab-010, Chain: HC
GAAGTGCAGCTGGTGGAGTCTGGGGGAGGCTTGGTACAGCCTGGGGGGTCCCTGAGACTCTCCTGTACATCCTCTGGATTCACCTTTGCTGATTATGCCATGCACTGGGTCCGGCAAACTCCAGGGAAGGGCCTGGAGTGGGTCTCAGGTATTAGTTGGAATGGTGGTACCTTTGACTATGCGGACTCTGTGAAGGGCCGATTCACCATCTCCAGAGACATCGCCAAGAACTCCCTGTATCTGCAAATGAACAGTCTGAGAGTTGAGGACACGGCCTTATATTATTGTGCAAAGGATATAGCATATAGCAGAGGATACTTCCAGCACTGGGGCCAGGGCACCCTGGTCACCGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 111 - protein, 645 aa, synthetic construct
  REGION 1..645: note = Description of sequence: Name: Ab-010, Chain: LC
GAAATTGTGTTGACGCAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCACCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCTCTCACTTTCGGCGGAGGGACCAAGGTGGAGATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 112 - protein, 11 aa, synthetic construct
  REGION 1..11: note = Description of sequence: Name: Ab-012, Chain: LCDR1
RASQSVSSNLA

SEQ ID NO: 113 - protein, 9 aa, synthetic construct
  REGION 1..9: note = Description of sequence: Name: Ab-012, Chain: LCDR3
QQYGSSQIT

SEQ ID NO: 114 - protein, 120 aa, synthetic construct; identical to SEQ ID NO: 27
  REGION 1..120: note = Description of sequence: Name: Ab-012, Chain: VH

SEQ ID NO: 115 - protein, 107 aa, synthetic construct
  REGION 1..107: note = Description of sequence: Name: Ab-012, Chain: VL
EIVLTQSPATLSVSPGERATLSCRASQSVSSNLAWYQQKPGQAPRLLIYGASSRATGIPDRFSGSGSGTDFTLTISRLEPEDFAVYYCQQYGSSQITFGQGTRLEIK

SEQ ID NO: 116 - protein, 360 aa, synthetic construct; identical to SEQ ID NO: 85
  REGION 1..360: note = Description of sequence: Name: Ab-012, Chain: VH

SEQ ID NO: 117 - protein, 321 aa, synthetic construct
  REGION 1..321: note = Description of sequence: Name: Ab-012, Chain: VL
GAAATTGTGTTGACGCAGTCTCCAGCCACCCTGTCTGTGTCTCCAGGGGAAAGAGCCACCCTCTCCTGCAGGGCCAGTCAGAGTGTTAGCAGCAACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACAGATCACCTTCGGCCAAGGGACACGACTGGAGATTAAA

SEQ ID NO: 118 - protein, 449 aa, synthetic construct; identical to SEQ ID NO: 31
  REGION 1..449: note = Description of sequence: Name: Ab-012, Chain: HC

SEQ ID NO: 119 - protein, 214 aa, synthetic construct
  REGION 1..214: note = Description of sequence: Name: Ab-012, Chain: LC
EIVLTQSPATLSVSPGERATLSCRASQSVSSNLAWYQQKPGQAPRLLIYGASSRATGIPDRFSGSGSGTDFTLTISRLEPEDFAVYYCQQYGSSQITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 1347 aa, synthetic construct; identical to SEQ ID NO: 89
  REGION 1..1347: note = Description of sequence: Name: Ab-012, Chain: HC

SEQ ID NO: 121 - protein, 642 aa, synthetic construct
  REGION 1..642: note = Description of sequence: Name: Ab-012, Chain: LC
GAAATTGTGTTGACGCAGTCTCCAGCCACCCTGTCTGTGTCTCCAGGGGAAAGAGCCACCCTCTCCTGCAGGGCCAGTCAGAGTGTTAGCAGCAACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACAGATCACCTTCGGCCAAGGGACACGACTGGAGATTAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 122 - protein, 120 aa, synthetic construct; identical to SEQ ID NO: 94
  REGION 1..120: note = Description of sequence: Name: Ab-014, Chain: VH

SEQ ID NO: 123 - protein, 108 aa, synthetic construct
  REGION 1..108: note = Description of sequence: Name: Ab-014, Chain: VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVDIK

SEQ ID NO: 124 - protein, 360 aa, synthetic construct
  REGION 1..360: note = Description of sequence: Name: Ab-014, Chain: VH
GAAGTGCAGCTGGTGGAGTCTGGGGGAGGCTTGGTACAGCCTGGCAGGTCCCTGAGACTCTCCTGTACAACCTCTGGATTCACCTTTGCTGATTATGCCATGCACTGGGTCCGGCAAACTCCAGGGAAGGGCCTGGAGTGGGTCTCAGGTATTAGTTGGAATGGTGTTACCATTGACTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATAGCATATAGCAGAGGATACTTCCAGCACTGGGGCCAGGGCACCCTGGTCACCGTCTCTTCA

SEQ ID NO: 125 - protein, 324 aa, synthetic construct
  REGION 1..324: note = Description of sequence: Name: Ab-014, Chain: VL
GAAATTGTGTTGACGCAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCGCTCACTTTCGGCGGAGGGACCAAAGTGGATATCAAA

SEQ ID NO: 126 - protein, 449 aa, synthetic construct; identical to SEQ ID NO: 98
  REGION 1..449: note = Description of sequence: Name: Ab-014, Chain: HC

SEQ ID NO: 127 - protein, 215 aa, synthetic construct
  REGION 1..215: note = Description of sequence: Name: Ab-014, Chain: LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 1347 aa, synthetic construct
  REGION 1..1347: note = Description of sequence: Name: Ab-014, Chain: HC
GAAGTGCAGCTGGTGGAGTCTGGGGGAGGCTTGGTACAGCCTGGCAGGTCCCTGAGACTCTCCTGTACAACCTCTGGATTCACCTTTGCTGATTATGCCATGCACTGGGTCCGGCAAACTCCAGGGAAGGGCCTGGAGTGGGTCTCAGGTATTAGTTGGAATGGTGTTACCATTGACTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATAGCATATAGCAGAGGATACTTCCAGCACTGGGGCCAGGGCACCCTGGTCACCGTCTCTTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 129 - protein, 645 aa, synthetic construct
  REGION 1..645: note = Description of sequence: Name: Ab-014, Chain: LC
GAAATTGTGTTGACGCAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCGCTCACTTTCGGCGGAGGGACCAAAGTGGATATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 130 - protein, 9 aa, synthetic construct
  REGION 1..9: note = Description of sequence: Name: Ab-016, Chain: LCDR3
QQYGSSPHT

SEQ ID NO: 131 - protein, 120 aa, synthetic construct
  REGION 1..120: note = Description of sequence: Name: Ab-016, Chain: VH
EVQLVESGGGLVQPGRSLRLSCTTSGFTFADYAMHWVRQTPGKGLEWVSGISWNGGTIGYADSVKGRFTISRDIAKNSLYLQMNSLRAEDTALYYCAKDIAYSRGYFQHWGQGTLVTVSS

SEQ ID NO: 132 - protein, 108 aa, synthetic construct
  REGION 1..108: note = Description of sequence: Name: Ab-016, Chain: VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPHTFGQGTKVEIK

SEQ ID NO: 133 - protein, 360 aa, synthetic construct
  REGION 1..360: note = Description of sequence: Name: Ab-016, Chain: VH
GAAGTGCAGCTGGTGGAGTCTGGGGGAGGCTTGGTACAGCCTGGCAGGTCCCTGAGACTCTCCTGTACAACCTCTGGATTCACCTTTGCTGATTATGCCATGCACTGGGTCCGGCAAACTCCAGGGAAGGGCCTGGAGTGGGTCTCAGGTATTAGTTGGAATGGTGGTACCATAGGCTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATAGCATATAGCAGAGGATACTTCCAGCACTGGGGCCAGGGCACCCTGGTCACCGTCTCCTCA

SEQ ID NO: 134 - protein, 324 aa, synthetic construct
  REGION 1..324: note = Description of sequence: Name: Ab-016, Chain: VL
GAAATTGTGTTGACGCAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCTCACACTTTTGGCCAGGGGACCAAGGTGGAAATCAAA

SEQ ID NO: 135 - protein, 449 aa, synthetic construct
  REGION 1..449: note = Description of sequence: Name: Ab-016, Chain: HC
EVQLVESGGGLVQPGRSLRLSCTTSGFTFADYAMHWVRQTPGKGLEWVSGISWNGGTIGYADSVKGRFTISRDIAKNSLYLQMNSLRAEDTALYYCAKDIAYSRGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 136 - protein, 215 aa, synthetic construct
  REGION 1..215: note = Description of sequence: Name: Ab-016, Chain: LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 1347 aa, synthetic construct
  REGION 1..1347: note = Description of sequence: Name: Ab-016, Chain: HC
GAAGTGCAGCTGGTGGAGTCTGGGGGAGGCTTGGTACAGCCTGGCAGGTCCCTGAGACTCTCCTGTACAACCTCTGGATTCACCTTTGCTGATTATGCCATGCACTGGGTCCGGCAAACTCCAGGGAAGGGCCTGGAGTGGGTCTCAGGTATTAGTTGGAATGGTGGTACCATAGGCTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATAGCATATAGCAGAGGATACTTCCAGCACTGGGGCCAGGGCACCCTGGTCACCGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 138 - protein, 645 aa, synthetic construct
  REGION 1..645: note = Description of sequence: Name: Ab-016, Chain: LC
GAAATTGTGTTGACGCAGTCTCCAGGCACCCTGTCTTTGTCTCCAGGGGAAAGAGCCACCCTCTCCTGCAGGGCCAGTCAGAGTGTTAGCAGCAGCTACTTAGCCTGGTACCAGCAGAAACCTGGCCAGGCTCCCAGGCTCCTCATCTATGGTGCATCCAGCAGGGCCACTGGCATCCCAGACAGGTTCAGTGGCAGTGGGTCTGGGACAGACTTCACTCTCACCATCAGCAGACTGGAGCCTGAAGATTTTGCAGTGTATTACTGTCAGCAGTATGGTAGCTCACCTCACACTTTTGGCCAGGGGACCAAGGTGGAAATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 139 - protein, 120 aa, synthetic construct; identical to SEQ ID NO: 27
  REGION 1..120: note = Description of sequence: Name: Ab-017, Chain: VH

SEQ ID NO: 140 - protein, 108 aa, synthetic construct; identical to SEQ ID NO: 123
  REGION 1..108: note = Description of sequence: Name: Ab-017, Chain: VL

SEQ ID NO: 141 - protein, 360 aa, synthetic construct
  REGION 1..360: note = Description of sequence: Name: Ab-017, Chain: VH
GAAGTGCAGCTGGTGGAGTCTGGGGGAGGCTTGGTACAGCCTGGCAGGTCCCTGAGACTCTCCTGTACAACCTCTGGATTCACCTTTGCTGATTATGCCATGCACTGGGTCCGGCAAACTCCAGGGCAGGGCCTGGAGTGGGTCTCAGGTATTAGTTGGAATGGTGGTACCATGGGCTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATCGCATATAGCAGAGGATACTTCCAGCACTGGGGCCAGGGCACCCTGGTCACTGTCTCCTCA

SEQ ID NO: 142 - protein, 324 aa, synthetic construct; identical to SEQ ID NO: 125
  REGION 1..324: note = Description of sequence: Name: Ab-017, Chain: VL

SEQ ID NO: 143 - protein, 449 aa, synthetic construct; identical to SEQ ID NO: 31
  REGION 1..449: note = Description of sequence: Name: Ab-017, Chain: HC

SEQ ID NO: 144 - protein, 215 aa, synthetic construct; identical to SEQ ID NO: 127
  REGION 1..215: note = Description of sequence: Name: Ab-017, Chain: LC

SEQ ID NO: 145 - protein, 1347 aa, synthetic construct
  REGION 1..1347: note = Description of sequence: Name: Ab-017, Chain: HC
GAAGTGCAGCTGGTGGAGTCTGGGGGAGGCTTGGTACAGCCTGGCAGGTCCCTGAGACTCTCCTGTACAACCTCTGGATTCACCTTTGCTGATTATGCCATGCACTGGGTCCGGCAAACTCCAGGGCAGGGCCTGGAGTGGGTCTCAGGTATTAGTTGGAATGGTGGTACCATGGGCTATGCGGACTCTGTGAAGGGCCGATTCACCATCTCCAGAGACATCGCCAAGAACTCCCTGTATCTGCAAATGAACAGTCTGAGAGCTGAGGACACGGCCTTATATTATTGTGCAAAGGATATCGCATATAGCAGAGGATACTTCCAGCACTGGGGCCAGGGCACCCTGGTCACTGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 146 - protein, 645 aa, synthetic construct; identical to SEQ ID NO: 129
  REGION 1..645: note = Description of sequence: Name: Ab-017, Chain: LC